(19) United States
(12) Patent Application Publication
(10) Pub. No.: US 2017/0121730 A1
(43) Pub. Date: May 4, 2017

(54) PLANTS HAVING ALTERED AGRONOMIC CHARACTERISTICS UNDER ABIOTIC CONDITIONS AND RELATED CONSTRUCTS AND METHODS INVOLVING ABIOTIC TOLERANCE GENES

(71) Applicant: PIONEER OVERSEAS CORPORATION, Johnston, IA (US)

(72) Inventors: GUIHUA LU, Haidian district, Beijing (CN); YANG GAO, Haidian district, Beijing (CN); MIN LIU, Haidian District, Beijing (CN); GUANFAN MAO, Haidian District, Beijing (CN); CHANGGUI WANG, Haidian District, Beijing (CN); WEI WANG, Changping district, Beijing (CN); XIPING WANG, Chaoyang district, Beijing (CN)

(21) Appl. No.: 15/318,524

(22) PCT Filed: Jul. 2, 2015
(86) PCT No.: PCT/CN2015/083234

§ 371 Date: Dec. 13, 2016

(30) Foreign Application Priority Data
Jul. 3, 2014 (CN) PCT/CN2014/081603

(51) Int. Cl. C12N 15/82; C12N 9/10; C07K 14/415

(52) U.S. Cl. CPC C12N 15/8273; C12N 15/8218; C07K 14/415; C12N 9/1088; C12Y 205/01018

(57) ABSTRACT

Isolated polynucleotides and polypeptides, and recombinant DNA constructs useful for conferring improved drought tolerance and/or cold tolerance; compositions (such as plants or seeds) comprising these recombinant DNA constructs; and methods utilizing these recombinant DNA constructs are disclosed. The recombinant DNA constructs comprise a polynucleotide operably linked to a promoter that is functional in a plant, wherein said polynucleotides encode drought tolerance polypeptides and/or cold tolerance polypeptides.

### FIELD

The field of the disclosure relates to plant breeding and genetics and, in particular, relates to recombinant DNA constructs useful in plants for improving tolerance to abiotic stress, such as drought, and cold stress.

### BACKGROUND

Stresses to plants may be caused by both biotic and abiotic agents. For example, biotic causes of stress include infection with pathogen, insect feeding, and parasitism by another plant such as mistletoe. Abiotic stresses include, for example, excessive or insufficient available water, temperature extremes, and synthetic chemicals such as herbicides.

Abiotic stress is the primary cause of crop loss worldwide, causing average yield losses more than 50% for major crops (Boyer, J.S. (1982) Science 218:443-448; Bray, E. A. et al. (2000) In Biochemistry and Molecular Biology of Plants, edited by Buchannan, B. B. et al., Amer. Soc. Plant Biol., pp. 1158-1249). Plants are sessile and have to adjust to the prevailing environmental conditions of their surroundings. This has led to their development of a great plasticity in gene regulation, morphogenesis, and metabolism. Adaption and defense strategies involve the activation of genes encoding proteins important in the acclimation or defense towards the different stresses.

Drought (insufficient available water) is one of the major abiotic stresses that limit crop productivity worldwide, and exposure of plants to a water-limiting environment during various developmental stages appears to activate various physiological and developmental changes. Although many reviews on molecular mechanisms of abiotic stress responses and genetic regulatory networks of drought stress tolerance have been published (Valliyodan, B., and Nguyen, H. T. (2006) Curr. Opin. Plant Biol. 9:189-195; Wang, W., et al. (2003) Planta 218:1-14; Vinocur, B., and Altman, A. (2005) Curr. Opin. Biotechnol.16:123-132; Chaves, M. M., and Oliveira, M. M. (2004) J. Exp. Bot. 55:2365-2384; Shinozaki, K., et al. (2003) Curr. Opin. Plant Biol. 6:410-417; Yamaguchi-Shinozaki, K., and Shinozaki, K. (2005) Trends Plant Sci. 10:88-94), it remains a major challenge in biology to understand the basic biochemical and molecular mechanisms for drought stress perception, signal transduction and tolerance. Genetic research has shown that drought tolerance is a quantitative trait, controlled by many genes. Molecular marker-assisted breeding has led to improved drought tolerance in crops. However, marker accuracy and breeding efficiency remain problematic (Ashraf M. (2010) Biotechnol. Adv. 28:169-183). Transgenic approaches to engineering drought tolerance in crops have made progress (Vinocur B. and Altman A. (2005) Curr. Opin. Biotechnol. 16: 123-132; Lawlor D W. (2013) J. Exp. Bot. 64:83-108).

Cold (low temperatures) can also reduce crop production. A sudden frost in spring or fall may cause premature tissue death.

Physiologically, the effects of drought and low temperature stress may be similar, as both result in cellular dehydration. For example, ice formation in the intercellular spaces draws water across the plasma membrane, creating a water deficit within the cell. Thus, improvement of a plant's drought tolerance may improve its cold tolerance as well.

Earlier work on molecular aspects of abiotic stress responses was accomplished by differential and/or subtractive analysis (Bray, E. A. (1993) Plant Physiol. 103:1035-1040; Shinozaki, K., and Yamaguchi-Shinozaki, K. (1997) Plant Physiol. 115:327-334; Zhu, J.-K. et al. (1997) Crit. Rev. Plant Sci. 16:253-277; Thomashow, M. F. (1999) Annu. Rev. Plant Physiol. Plant Mol. Biol. 50:571-599); and other methods which include selection of candidate genes and analysis of expression of such a gene or its active product under stresses, or by functional complementation in a stressor system that is well defined (Xiong, L. and Zhu, J.-K. (2001) Physiologia Plantarum 112:152-166). Additionally, forward and reverse genetic studies involving the identification and isolation of mutations in regulatory genes have been used to provide evidence for observed changes in gene expression under stress (Xiong, L. and Zhu, J.-K. (2001) Physiologia Plantarum 112:152-166).

Activation tagging can be utilized to identify genes with the ability to affect a trait,and this approach has been used in Arabidopsis thaliana (the model plant species) (Weigel, D., et al. (2000) Plant Physiol. 122:1003-1013). Insertions of transcriptional enhancer elements can dominantly activate and/or elevate the expression of nearby endogenous genes, so this method can be used to select genes involved in agronomically important phenotypes, including abiotic stress tolerance such as improved drought tolerance and cold tolerance.

### SUMMARY

The following embodiments are among those encompassed by the disclosure:

In one embodiment, the present disclosure includes an isolated polynucleotide, comprising: (a) a polynucleotide with nucleotide sequence of at least 85% sequence identity, based on the Clustal V method of alignment, to SEQ ID NO: 3, 6, 9, 12, 15, 18or 21; (b) a polynucleotide with nucleotide sequence of at least 85% sequence identity, based on the Clustal V method of alignment, to SEQ ID NO: 4, 7, 10, 13, 16, 19 or 22;(c) a polynucleotide encoding a polypeptide with amino acid sequence of at least 90% sequence identity, based on the Clustal V method of alignment, to SEQ ID NO: 5, 8, 11, 14, 17, 20 or 23; or(d) the full complement of the nucleotide sequence of (a), (b) or (c), wherein over-expression of the polynucleotide in a plant enhances drought tolerance;the isolated polynucleotide comprises the nucleotide sequence of SEQ ID NO: 3, 4, 6, 7, 9, 10, 12, 13, 15, 16, 18, 19, 21 or 22;and the said polypeptide comprises the amino acid sequence of SEQ ID NO: 5, 8, 11, 14, 17, 20 or 23.

In another embodiment, the present disclosure includes a recombinant DNA construct comprising the isolated polynucleotide operably linked to at least one heterologous regulatory sequence, wherein the polynucleotide comprises (a) a polynucleotide with nucleotide sequence of at least 85% sequence identity, based on the Clustal V method of alignment, to SEQ ID NO: 3, 4, 6, 7, 9, 10, 12, 13, 15, 16, 18, 19, 21or 22; (b) a polynucleotide encoding a polypeptide with amino acid sequence of at least 90% sequence identity, based on the Clustal V method of alignment, to SEQ ID NO: 5, 8, 11, 14, 17, 20 or 23; or(c) the full complement of the nucleotide sequence of (a) or (b).

In another embodiment, the present disclosure includes a transgenic plant or seed comprising a recombinant DNA construct, wherein the recombinant DNA construct comprises the polynucleotide operably linked to at least one regulatory sequence, wherein the polynucleotide comprises (a) a polynucleotide with nucleotide sequence of at least 85% sequence identity, based on the Clustal V method of alignment, to SEQ ID NO: 3, 4, 6, 7, 9, 10, 12, 13, 15, 16, 18, 19, 21or 22; (b) a polynucleotide encoding a polypeptide with amino acid sequence of at least 90% sequence identity, based on the Clustal V method of alignment, to SEQ ID NO: 5, 8, 11, 14, 17, 20 or 23; or(c) the full complement of the nucleotide sequence of (a) or (b).

In another embodiment, the present disclosure includes a transgenic plant comprising in its genome a recombinant DNA construct comprising a polynucleotide operably linked to at least one regulatory element, wherein the polynucleotide comprises (a) a polynucleotide with nucleotide sequence of at least 85% sequence identity, based on the Clustal V method of alignment, to SEQ ID NO: 3, 4, 6, 7, 9, 10, 12, 13, 15, 16, 18, 19, 21or 22; (b) a polynucleotide encoding a polypeptide with amino acid sequence of at least 90% sequence identity, based on the Clustal V method of alignment, to SEQ ID NO: 5, 8, 11, 14, 17, 20 or 23; or(c) the full complement of the nucleotide sequence of (a) or (b); the said plant exhibits improved drought tolerance when compared to a control plant.

In another embodiment, the present disclosure includes any of the plants of the disclosure, wherein the plant is selected from the group consisting of rice, maize, soybean, sunflower, sorghum, canola, wheat, alfalfa, cotton, barley, millet, sugar cane and switchgrass.

In another embodiment, methods are provided for increasing drought tolerance in a plant, comprising: (a) introducing into a regenerable plant cell a recombinant DNA construct comprising a polynucleotide operably linked to at least one regulatory sequence, wherein the polynucleotide encodes a polypeptide having an amino acid sequence of at least 50% sequence identity, when compared to SEQ ID NO: 5, 8, 11, 14, 17, 20 or 23; (b) regenerating a transgenic plant from the regenerable plant cell after step (a), wherein the transgenic plant comprises in its genome the recombinant DNA construct; and (c) obtaining a progeny plant derived from the transgenic plant of step (b), wherein said progeny plant comprises in its genome the recombinant DNA construct and exhibits increased drought tolerance when compared to a control plant not comprising the recombinant DNA construct.

In another embodiment, methods are provided for evaluating drought tolerance in a plant, comprising: (a) introducing into a regenerable plant cell a recombinant DNA construct comprising a polynucleotide operably linked to at least one regulatory sequence, wherein the polynucleotide encodes a polypeptide having an amino acid sequence of at least 50% sequence identity, when compared to SEQ ID NO: 5, 8, 11, 14, 17, 20 or 23; (b) regenerating a transgenic plant from the regenerable plant cell after step (a), wherein the transgenic plant comprises in its genome the recombinant DNA construct; (c) obtaining a progeny plant derived from the transgenic plant, wherein the progeny plant comprises in its genome the recombinant DNA construct; and (d) evaluating the progeny plant for drought tolerance compared to a control plant not comprising the recombinant DNA construct.

In one embodiment, the present disclosure includes an isolated polynucleotide, comprising: (a) a polynucleotide with nucleotide sequence of at least 85% sequence identity, based on the Clustal V method of alignment, to SEQ ID NO:18or 27; (b) a polynucleotide with nucleotide sequence of at least 85% sequence identity, based on the Clustal V method of alignment, to SEQ ID NO:19 or 28; (c) a polynucleotide encoding a polypeptide with amino acid sequence of at least 90% sequence identity, based on the Clustal V method of alignment, to SEQ ID NO: 20 or 29; or(d) the full complement of the nucleotide sequence of (a), (b) or (c), wherein over-expression of the polynucleotide in a plant enhances cold tolerance; the isolated polynucleotide comprises the nucleotide sequence of SEQ ID NO: 18, 19, 27 or 28; and the said polypeptide comprises the amino acid sequence of SEQ ID NO: 20 or 29.

In another embodiment, the present disclosure includes a recombinant DNA construct comprising the isolated polynucleotide operably linked to at least one heterologous regulatory sequence, wherein the polynucleotide comprises (a) a polynucleotide with nucleotide sequence of at least 85% sequence identity, based on the Clustal V method of alignment, to SEQ ID NO:18, 19, 27or 28; (b) a polynucleotide encoding a polypeptide with amino acid sequence of at least 90% sequence identity, based on the Clustal V method of alignment, to SEQ ID NO: 20 or 29; or(c) the full complement of the nucleotide sequence of (a) or (b).

In another embodiment, the present disclosure includes a transgenic plant or seed comprising a recombinant DNA construct, wherein the recombinant DNA construct comprises the polynucleotide operably linked to at least one regulatory sequence, wherein the polynucleotide comprises (a) a polynucleotide with nucleotide sequence of at least 85% sequence identity, based on the Clustal V method of alignment, to SEQ ID NO: 18, 19, 27or 28; (b) a polynucleotide encoding a polypeptide with amino acid sequence of at least 90% sequence identity, based on the Clustal V method of alignment, to SEQ ID NO: 20 or 29; or (c) the full complement of the nucleotide sequence of (a) or (b).

In another embodiment, the present disclosure includes a transgenic plant comprising in its genome a recombinant DNA construct comprising a polynucleotide operably linked to at least one regulatory element, wherein the polynucleotide comprises (a) a polynucleotide with nucleotide sequence of at least 85% sequence identity, based on the Clustal V method of alignment, to SEQ ID NO: 18, 19, 27or 28; (b) a polynucleotide encoding a polypeptide with amino acid sequence of at least 90% sequence identity, based on the Clustal V method of alignment, to SEQ ID NO: 20 or 29; or(c) the full complement of the nucleotide sequence of (a) or (b); the said plant exhibits improved cold tolerance when compared to a control plant.

In another embodiment, methods are provided for increasing cold tolerance in a plant, comprising: (a) introducing into a regenerable plant cell a recombinant DNA construct comprising a polynucleotide operably linked to at least one regulatory sequence, wherein the polynucleotide encodes a polypeptide having an amino acid sequence of at least 50% sequence identity, when compared to SEQ ID NO: 20 or 29; (b)regenerating a transgenic plant from the regenerable plant cell after step (a), wherein the transgenic plant comprises in its genome the recombinant DNA construct; and (c)obtaining a progeny plant derived from the transgenic plant of step (b), wherein said progeny plant comprises in its genome the recombinant DNA construct and exhibits increased cold tolerance when compared to a control plant not comprising the recombinant DNA construct.

In another embodiment, methods are provided for evaluating cold tolerance in a plant, comprising: (a)introducing into a regenerable plant cell a recombinant DNA construct comprising a polynucleotide operably linked to at least one regulatory sequence, wherein the polynucleotide encodes a polypeptide having an amino acid sequence of at least 50% sequence identity, when compared to SEQ ID NO: 20 or 29; (b)regenerating a transgenic plant from the regenerable plant cell after step (a), wherein the transgenic plant comprises in its genome the recombinant DNA construct; (c)obtaining a progeny plant derived from the transgenic plant, wherein the progeny plant comprises in its genome the recombinant DNA construct; and (d)evaluating the progeny plant for cold tolerance compared to a control plant not comprising the recombinant DNA construct.

In another embodiment, the present disclosure concerns a recombinant DNA construct comprising any of the isolated polynucleotides of the present disclosure operably linked to at least one regulatory sequence, and a cell, a plant, or a seed comprising the recombinant DNA construct. The cell may be eukaryotic, e.g., a yeast, insect or plant cell; or prokaryotic, e.g., a bacterial cell.

### BRIEF DESCRIPTION OF THE DRAWINGS AND SEQUENCE LISTING

The disclosure can be more fully understood from the following detailed description and the accompanying drawings and Sequence Listing which form a part of this application.

FIG. 1 shows changes of soil volumetric moisture content at different developmental stage in Hainan field in the first field experiment for drought testing OsDN-DTP2-transgenic rice. The OsDN-DTP2-transgenic rice started heading at 22 days after stopping watering and matured at 60 days after stopping watering.

FIG. 2 shows changes of soil volumetric moisture content at different developmental stage in Beijing field in the first field experiment for drought testing OsBCS1L-transgenic rice. The OsBCS1L-transgenic rice started heading at 47 days after stopping watering and matured at 86 days after stopping watering.

FIG. 3 provides relative expression levels by real-time PCR analyses of OsBCS1L transgene in leaves of separate transgenic rice events which were drought treated. The base level of expression in ZH11-TC was set at 1.00, and the expression levels in other OsBCS1L events were shown as fold-increases compared to ZH11-TC. DP0196-BN represents the rice plants segregated from hemizygous OsBCS1L-transgenic events.

Table 1.SEQ ID NOs for nucleotide and amino acid sequences provided in the sequence listing

Table 2. Rice gene names, Gene IDs (from TIGR) and Construct IDs

Table 3. Primers for cloning rice drought tolerance genes and cold tolerance genes

Table 4. PCR reaction mixture for cloning drought tolerance genes and cold tolerance genes

Table 5. PCR cycle conditions

Table 6. Enhanced drought tolerance of OsDN-DTP2-transgenic rice plants at T₂ generation under greenhouse conditions

Table 7. Enhanced drought tolerance of OsMRP10-transgenic rice plants at T₂ generation under greenhouse conditions (1^{st} experiment)

Table 8. Enhanced drought tolerance of OsMRP10-transgenic rice plants at T₂ generation under greenhouse conditions at construct level (2^{nd} experiment)

Table 9. Enhanced drought tolerance of OsGSTU35-transgenic rice plants at T₂ generation under greenhouse conditions (1^{st} experiment)

Table 10. Enhanced drought tolerance of OsGSTU35-transgenic rice plants at T₂ generation under greenhouse conditions at construct level (2^{nd} experiment)

Table 11. Enhanced drought tolerance of OsCML1-transgenic rice plants at T₂ generation under greenhouse conditions

Table 12. Enhanced drought tolerance of OsIMPA1a-transgenic rice plants at T₂ generation under greenhouse conditions (1^{st} experiment)

Table 13. Enhanced drought tolerance of OsIMPA1a-transgenic rice plants at T₂ generation under greenhouse conditions at construct level (2^{nd} experiment)

Table 14. Enhanced drought tolerance of OsMYB125-transgenic rice plants at T2 generation under greenhouse conditions (1^{st} experiment)

Table 15. Enhanced drought tolerance of OsMYB125-transgenic rice plants at T₂ generation under greenhouse conditions (2^{nd} experiment)

Table 16. Enhanced drought tolerance of OsCML3-transgenic rice plants at T₂ generation under greenhouse conditions (1^{st} experiment)

Table 17. Enhanced drought tolerance of OsCML3-transgenic rice plants at T₂ generation under greenhouse conditions at construct level(2^{nd} experiment)

Table 18. Enhanced drought tolerance of OsBCS1L-transgenic rice plants at T₂ generation under greenhouse conditions (1^{st} experiment)

Table 19. Enhanced drought tolerance of OsBCS1L-transgenic rice plants at T₂ generation under greenhouse conditions at construct level (2^{nd} experiment)

Table 20. Grain yield assay of OsDN-DTP2-rice plants at T₂ generation under field drought conditions

Table 21. Grain yield assay of OsBCS1L-rice plants at T₂ generation under field drought conditions

Table 22. Enhanced cold tolerance of OsMYB125-transgenic rice plants at T₂ generation under low temperature

Table 23. Enhanced cold tolerance of OsDN-CTP1-transgenic rice plants at T₂ generation under low temperature

Table 24. Paraquat tolerance assay of OsDN-DTP2-transgenic rice plants at T₂ generation at transgenic event level

Table 25. Paraquat tolerance assay of OsGSTU35-transgenic rice plants at T₂ generation at transgenic event level

Table 26. Paraquat tolerance assay of OsCML1-transgenic rice plants at T₂ generation at transgenic event level

Table 27. Paraquat tolerance assay of OsIMPA1a-transgenic rice plants at T₂ generation at transgenic event level

Table 28. Paraquat tolerance assay of OsMYB125-transgenic rice plants at T₂ generation at transgenic event level

Table 29. Paraquat tolerance assay of OsBCS1L-transgenic rice plants at T₂ generation at transgenic event level

Table 30. Paraquat tolerance assay of OsDN-CTP1-transgenic rice plant at T₂ generation at transgenic event level

TABLE 1
SEQ ID NOs for nucleotide and amino acid sequences provided in
the sequence listing

| | | SEQ ID NO: | SEQ ID NO: |
|---|---|---|---|
| Source species | Clone Designation | (Nucleotide) | (Amino Acid) |
| Artificial | DP0158 vector | 1 | n/a |
| Artificial | DsRed expression cassette | 2 | n/a |
| Oryza sativa | OsDN-DTP2 | 3, 4 | 5 |
| Oryza sativa | OsMRP10 | 6, 7 | 8 |
| Oryza sativa | OsGSTU35 | 9, 10 | 11 |
| Oryza sativa | OsCML1 | 12, 13 | 14 |
| Oryza sativa | OsIMFA1a | 15, 16 | 17 |
| Oryza sativa | OsMYB125 | 18, 19 | 20 |
| Oryza sativa | OsCML3 | 21, 22 | 23 |
| Oryza sativa | OsBCS1L | 24, 25 | 26 |
| Oryza sativa | OsDN-CTP1 | 27, 28 | 29 |
| Artificial | Primers | 30-49 | n/a |

The Sequence Listing contains the one-letter code for nucleotide sequences and the three-letter code for amino acid sequences as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37C.F.R.§1.822.

SEQ ID NO: 1 is the nucleotide sequence of vector DP0005.

SEQ ID NO: 2 is the nucleotide sequence of DsRed expression cassette.

SEQ ID NO: 3 is the nucleotide sequence of gDNA of OsDN-DTP2 gene.

SEQ ID NO: 4 is the nucleotide sequence of CDS of OsDN-DTP2 gene.

SEQ ID NO: 5 is the amino acid sequence of OsDN-DTP2.

SEQ ID NO: 6 is the nucleotide sequence of gDNA of OsMRP10 gene.

SEQ ID NO: 7 is the nucleotide sequence of CDS of OsMRP10 gene.

SEQ ID NO: 8 is the amino acid sequence of OsMRP10.

SEQ ID NO: 9 is the nucleotide sequence of cDNA of OsGSTU35gene.

SEQ ID NO: 10 is the nucleotide sequence of CDS of OsGSTU35gene.

SEQ ID NO: 11 is the amino acid sequence of OsGSTU35.

SEQ ID NO: 12 is the nucleotide sequence of cDNA of OsCML1 gene.

SEQ ID NO: 13 is the nucleotide sequence of CDS of OsCML1 gene.

SEQ ID NO: 14 is the amino acid sequence of OsCML1.

SEQ ID NO: 15 is the nucleotide sequence of cDNA of OsIMPA1a gene.

SEQ ID NO: 16 is the nucleotide sequence of CDS of OsIMPA1a gene.

SEQ ID NO: 17 is the amino acid sequence of OsIMPA1a.

SEQ ID NO: 18 is the nucleotide sequence of cDNA of OsMYB125 gene.

SEQ ID NO: 19 is the nucleotide sequence of CDS of OsMYB125 gene.

SEQ ID NO: 20 is the amino acid sequence of OsMYB125.

SEQ ID NO: 21 is the nucleotide sequence of cDNA of OsCML3 gene.

SEQ ID NO: 22 is the nucleotide sequence of CDS of OsCML3 gene.

SEQ ID NO: 23 is the amino acid sequence of OsCML3.

SEQ ID NO: 24 is the nucleotide sequence of cDNA of OsBCS1L gene.

SEQ ID NO: 25 is the nucleotide sequence of CDS of OsBCS1L gene.

SEQ ID NO: 26 is the amino acid sequence of OsBCS1L.

SEQ ID NO: 27 is the nucleotide sequence of gDNA of OsDN-CTP1 gene.

SEQ ID NO: 28 is the nucleotide sequence of CDS of OsDN-CTP1 gene.

SEQ ID NO: 29 is the amino acid sequence of OsDN-CTP1.

SEQ ID NO: 30 is forward primer for cloning gDNA of OsDN-DTP2 gene.

SEQ ID NO: 31 is reverse primer for cloning gDNA of OsDN-DTP2 gene.

SEQ ID NO: 32 is forward primer for cloning gDNA of OsMRP10 gene.

SEQ ID NO: 33 is reverse primer for cloning gDNA of OsMRP10 gene.

SEQ ID NO: 34 is forward primer for cloning cDNA of OsGSTU35 gene.

SEQ ID NO: 35 is reverse primer for cloning cDNA of OsGSTU35 gene.

SEQ ID NO: 36 is forward primer for cloning cDNA of OsCML1 gene.

SEQ ID NO: 37 is reverse primer for cloning cDNA of OsCML1 gene.

SEQ ID NO: 38 is forward primer for cloning cDNA of OsIMPA1a gene.

SEQ ID NO: 39 is reverse primer for cloning cDNA of OsIMPA1a gene.

SEQ ID NO: 40 is forward primer for cloning cDNA of OsMYB125 gene.

SEQ ID NO: 41 is reverse primer for cloning cDNA of OsMYB125 gene.

SEQ ID NO: 42 is forward primer for cloning cDNA of OsCML3 gene.

SEQ ID NO: 43 is reverse primer for cloning cDNA of OsCML3 gene.

SEQ ID NO: 44 is forward primer for cloning cDNA of OsBCS1L gene.

SEQ ID NO: 45 is reverse primer for cloning cDNA of OsBCS1L gene.

SEQ ID NO: 46 is forward primer for cloning gDNA of OsDN-CTP1 gene.

SEQ ID NO: 47is reverse primer for cloning gDNA of OsDN-CTP1 gene.

SEQ ID NO: 48 is forward primer for real-time RT-PCR analysis of OsBCS1L gene.

SEQ ID NO: 49 is reverse primer for real-time RT-PCR analysis of OsBCS1L gene.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants; reference to “a cell” includes one or more cells and equivalents thereof known to those skilled in the art, and so forth.

As used herein:

The term “OsDN-DTP2 (drought tolerance protein 2)” refers to a rice polypeptide that confers drought tolerance phenotype and is encoded by the rice gene locus Os08g0552300. “DN-DTP2 polypeptide” refers herein to the OsDN-DTP2 polypeptide and its homologs from other organisms.

The OsDN-DTP2 polypeptide (SEQ ID NO: 5) is encoded by the coding sequence (CDS) (SEQ ID NO: 4) or nucleotide sequence (SEQ ID NO: 3) at rice gene locus Os08g0552300. This polypeptide is annotated as “hypothetical protein” in NCBI (on the world web atncbi.nlm.nih.gov), however does not have any prior assigned function.

The term “OsMRP10 (multidrug resistance-associated protein 10)” refers to a rice polypeptide that confers drought tolerance phenotype and is encoded by the rice gene locus LOC_Os04g13220.1. “MRP10 polypeptide” refers herein to the OsMRP10 polypeptide and its homologs from other organisms.

The OsMRP10 polypeptide (SEQ ID NO: 8) is encoded by the coding sequence (CDS) (SEQ ID NO: 7) or nucleotide sequence (SEQ ID NO: 6) at rice gene locus LOC_Os04g13220.1. This polypeptide is annotated as “ABC transporter family protein, putative, expressed” in TIGR (the Internet atplantbiologymsu.edu/index.shtml), and “Glutathione-conjugate transporter AtMRP4” in NCBI.

The term “OsGSTU35 (Glutathione S-transferase TAU35)” refers to a rice polypeptide that confers drought tolerance and is encoded by the rice gene locus LOC_Os01g72130.1. “GSTU35 polypeptide” refers herein to the OsGSTU35polypeptide and its homologs from other organisms.

The OsGSTU35 polypeptide (SEQ ID NO: 11) is encoded by the coding sequence (CDS) (SEQ ID NO: 10) or nucleotide sequence (SEQ ID NO: 9) at rice gene locus LOC_Os01g72130.1. This polypeptide is annotated as “Glutathione S-transferase, putative, expressed” in TIGR and “putative glutathione S-transferase” in NCBI, however does not have any prior assigned function.

The term “OsCML1 (calmodulin-like protein 1)” refers to a rice polypeptide that confers drought tolerance and is encoded by the rice gene locus LOC_Os01g72080.1. “CML1 polypeptide” refers herein to the OsCML1 polypeptide and its homologs from other organisms.

The OsCML1 polypeptide (SEQ ID NO: 14) is encoded by the coding sequence (CDS) (SEQ ID NO: 13) or nucleotide sequence (SEQ ID NO: 12) at rice gene locus LOC_Os01g72080.1. This polypeptide is annotated as “calmodulin-like protein 1, putative, expressed” in TIGR.

The term “OsIMPA1a (importin subunit alpha, putative, expressed)” is a truncated importin subunit alpha and refers to a rice polypeptide that confers drought tolerance phenotype and is encoded by the rice gene locus LOC_Os05g06350.1. “IMPA1a polypeptide” refers herein to the OsIMPA1 a polypeptide and its homologs from other organisms.

The OsIMPA1a polypeptide (SEQ ID NO: 17) is encoded by the coding sequence (CDS) (SEQ ID NO: 16) or nucleotide sequence (SEQ ID NO: 15) at rice gene locus LOC_Os05g06350.1.

The term “OsMYB125 (Myb-like DNA-binding domain containing protein 125)” refers to a rice polypeptide that confers drought and cold tolerance and is encoded by the rice gene locus LOC_Os05g41240.1. “MYB125 polypeptide” refers herein to the OsMYB125 polypeptide and its homologs from other organisms.

The OsMYB125 polypeptide (SEQ ID NO: 20) is encoded by the coding sequence (CDS) (SEQ ID NO: 19) or nucleotide sequence (SEQ ID NO: 18) at rice gene locus LOC_Os05g41240.1. This polypeptide is annotated as “Myb-like DNA-binding domain containing protein, putative, expressed” in TIGR.

The term “OsCML3 (Calmodulin-related calcium sensor protein 3)” refers to a rice polypeptide that confers drought tolerance and is encoded by the rice gene locus LOC_Os12g03816.1. “CML3 polypeptide” refers herein to the OsCML3 polypeptide and its homologs from other organisms.

The OsCML3 polypeptide (SEQ ID NO: 23) is encoded by the coding sequence (CDS) (SEQ ID NO: 22) or nucleotide sequence (SEQ ID NO: 21) at rice gene locus LOC_Os12g03816.1. This polypeptide is annotated as “OsCML3—Calmodulin-related calcium sensor protein” in TIGR and (Calmodulin like protein 3) NCBI.

The term “OsBCS1L (mitochondrial chaperone BCS1 like protein)” refers to a rice polypeptide that confers drought sensitive phenotype and is encoded by the rice gene locus LOC_Os05g51130.1. “BCS1 L polypeptide” refers herein to the OsBCS1 L polypeptide and its homologs from other organisms.

The OsBCS1 L polypeptide (SEQ ID NO: 26) is encoded by the coding sequence (CDS) (SEQ ID NO: 25) or nucleotide sequence (SEQ ID NO: 24) at rice gene locus LOC_Os05g51130.1. This polypeptide is annotated as “mitochondrial chaperone BCS1, putative, expressed” in TIGR.

The term “OsDN-CTP1 (cold tolerance protein 1)” refers to a rice polypeptide that confers cold tolerance and is encoded by the rice gene locus LOC_Os02g20150.1. “DN-CTP1 polypeptide” refers herein to the OsDN-CTP1 polypeptide and its homologs from other organisms.

The OsDN-CTP1 polypeptide (SEQ ID NO: 29) is encoded by the coding sequence (CDS) (SEQ ID NO: 28) or nucleotide sequence (SEQ ID NO: 27) at rice gene locus LOC_Os02g20150.1. This polypeptide is annotated as “hypothetical protein” in TIGR.

The terms “monocot” and “monocotyledonous plant” are used interchangeably herein. A monocot of the current disclosure includes plants of the Gramineae family.

The terms “dicot” and “dicotyledonous plant” are used interchangeably herein. A dicot of the current disclosure includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

An “Expressed Sequence Tag” (“EST”) is a DNA sequence derived from a cDNA library and therefore represents a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

The term “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar or nitrogen concentrations, or by the observation of the expression level of a gene or genes, or by agricultural observations such as osmotic stress tolerance or yield.

“Agronomic characteristic” is a measurable parameter including but not limited to: greenness, grain yield, growth rate, total biomass or rate of accumulation, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, tiller number, panicle size, early seedling vigor and seedling emergence under low temperature stress.

Increased biomass can be measured, for example, as an increase in plant height, plant total leaf area, plant fresh weight, plant dry weight or plant seed yield, as compared with control plants.

The ability to increase the biomass or size of a plant would have several important commercial applications. Crop cultivars may be developed to produce higher yield of the vegetative portion of the plant, to be used in food, feed, fiber, and/or biofuel.

Increased leaf size may be of particular interest. Increased leaf biomass can be used to increase production of plant-derived pharmaceutical or industrial products. Increased tiller number may be of particular interest and can be used to increase yield. An increase in total plant photosynthesis is typically achieved by increasing leaf area of the plant. Additional photosynthetic capacity may be used to increase the yield derived from particular plant tissue, including the leaves, roots, fruits or seed, or permit the growth of a plant under decreased light intensity or under high light intensity.

Modification of the biomass of another tissue, such as root tissue, may be useful to improve a plant's ability to grow under harsh environmental conditions, including drought or nutrient deprivation, because larger roots may better reach or take up water or nutrients.

For some ornamental plants, the ability to provide larger varieties would be highly desirable. For many plants, including fruit-bearing trees, trees that are used for lumber production, or trees and shrubs that serve as view or wind screens, increased stature provides improved benefits, such as in the forms of greater yield or improved screening.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

A “control” or “control plant” or “control plant cell” provides a reference point for measuring changes in phenotype of a subject plant or plant cell in which genetic alteration, such as transformation, has been effected as to a gene of interest. A subject plant or plant cell may be descended from a plant or cell so altered and will comprise the alteration.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to a condition or stimulus that would induce expression of the gene of interest; or (e) the subject plant or plant cell itself, under conditions in which the gene of interest is not expressed.

“Genome” as it applies to plant cells encompasses not only chromosomal DNA found within the nucleus, but also organelle DNA found within subcellular components (e.g., mitochondria, plastid) of the cell.

“Plant” includes reference to whole plants, plant organs, plant tissues, seeds and plant cells and progeny of the same. Plant cells include, without limitation, cells from seeds, suspension cultures, embryos, meristematic regions, callus tissues, leaves, roots, shoots, gametophytes, sporophytes, pollen, and microspores.

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct. A T₀ plant is directly recovered from the transformation and regeneration process. Progeny of T₀ plants are referred to as Tᵢ (first progeny generation), T₂ (second progeny generation), etc.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, and “nucleic acid fragment” are used interchangeably and refer to a polymer of RNA or DNA that is single- or double-stranded, optionally containing synthetic, non-natural or altered nucleotide bases. Nucleotides (usually found in their 5′-monophosphate form) are referred to by their single-letter designation as follows: “A” for adenylate or deoxyadenylate, “C” for cytidylate or deoxycytidylate, and “G” for guanylate or deoxyguanylate for RNA or DNA, respectively; “U” for uridylate; “T” for deoxythymidylate; “R” for purines (A or G); “Y” for pyrimidines (C or T); “K” for G or T; “H” for A or C or T; “I” for inosine; and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, and sulfation, gamma-carboxylation of glutamic acid residues, hydroxylation and ADP-ribosylation.

“Messenger RNA (mRNA)” refers to the RNA which has no intron and can be translated into protein by the cell.

“cDNA” refers to a DNA that is complementary to and synthesized from an mRNA template using reverse transcriptase. The cDNA can be single-stranded or converted into the double-stranded form using the Klenow fragment of DNA polymerase I.

“Mature” protein refers to a post-translationally processed polypeptide; i.e., any pre- or pro-peptides present in the primary translation product has been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterogonous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature.

The terms “entry clone” and “entry vector” are used interchangeably herein.

“Regulatory sequences” refer to nucleotide sequences located upstream (5′ non-coding sequences), within, or downstream (3′ non-coding sequences) of a coding sequence, and influencing the transcription, RNA processing or stability, or translation of the associated coding sequence. Regulatory sequences may include, but are not limited to, promoters, translation leader sequences, introns, and poly-adenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription of genes in plant cells whether or not its origin is from a plant cell.

“Tissue-specific promoter” and “tissue-preferred promoter” may refer to a promoter that is expressed predominantly but not necessarily exclusively in one tissue or organ, but that may also be expressed in one specific cell or cell type.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

An “allele” is one of two or more alternative forms of a gene occupying a given locus on a chromosome. When the alleles present at a given locus on a pair of homologous chromosomes in a diploid plant are the same, that plant is homozygous at that locus. If the alleles present at a given locus on a pair of homologous chromosomes in a diploid plant differ, that plant is heterozygous at that locus. If a transgene is present on one of a pair of homologous chromosomes in a diploid plant, that plant is hemizygous at that locus.

A “chloroplast transit peptide” is an amino acid sequence which is translated in conjunction with a protein and directs the protein to the chloroplast or other plastid types present in the cell in which the protein is made. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels. (1991) Ann. Rev. Plant Phys. Plant Mol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel. (1992) Plant Phys. 100:1627-1632). A “mitochondrial signal peptide” is an amino acid sequence which directs a precursor protein into the mitochondria (Zhang and Glaser. (2002) Trends Plant Sci 7:14-21).

Methods to determine the relationship of various polynucleotide and polypeptide sequences are known. As used herein, “reference sequence” is a defined sequence used as a basis for sequence comparison. A reference sequence may be a subset or the entirety of a specified sequence, such as a segment of a full-length cDNA or gene sequence, or may be the complete cDNA or gene sequence. As used herein, “comparison window” makes reference to a contiguous and specified segment of a polynucleotide or polypeptide sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides or amino acids in length, and optionally can be 30, 40, 50, 100 or longer. Those of skill in the art understand that to avoid a high similarity to a reference sequence due to inclusion of gaps in the sequence, a gap penalty is typically introduced and is subtracted from the number of matches.

The determination of percent sequence identity between any two sequences can be accomplished using a mathematical algorithm. Examples of such mathematical algorithms for sequence comparison include the algorithm of Myers and Miller.(1988) CABIOS 4:11-17; the local alignment algorithm of Smith, et al. (1981) Adv. Appl. Math. 2:482; the global alignment algorithm of Needleman and Wunsch. (1970) J. Mol. Biol. 48:443-453; the search-for-local alignment method of Pearson and Lipman. (1988) Proc. Natl. Acad. Sci. 85:2444-2448; the algorithm of Karlin and Altschul. (1990) Proc. Natl. Acad. Sci. USA 872264, modified as in Karlin and Altschul. (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA and TFASTA in the GCG Wisconsin Genetics Software Package, Version 10 (available from Accelrys Inc., 9685 Scranton Road, San Diego, Calif., USA); and the Megalign® program of the LASERGENE® bioinformatics computing suite (DNASTAR® Inc., Madison, Wis.).

Alignments using these programs can be performed using the default parameters. The CLUSTAL program is well described by Higgins, et al. (1988) Gene 73:237-244; Higgins, et al. (1989) CABIOS 5:151-153; Corpet, et al. (1988) Nucleic Acids Res. 16:10881-10890; Huang, et al. (1992) CABIOS 8:155-165 and Pearson, et al. (1994) Meth. Mol. Biol. 24:307-331. The ALIGN program is based on the algorithm of Myers and Miller, (1988) supra. A PAM120 weight residue table, a gap length penalty of 12 and a gap penalty of 4 can be used with the ALIGN program when comparing amino acid sequences. The BLAST programs of Altschul, et al. (1990) J. Mol. Biol. 215:403 are based on the algorithm of Karlin and Altschul. (1990) supra. BLAST nucleotide searches can be performed with the BLASTN program, score=100, wordlength=12, to obtain nucleotide sequences homologous to a nucleotide sequence encoding a protein of the disclosures. BLAST protein searches can be performed with the BLASTX program, score=50, wordlength=3, to obtain amino acid sequences homologous to a protein or polypeptide of the disclosures. To obtain gapped alignments for comparison purposes, Gapped BLAST (in BLAST 2.0) can be utilized as described in Altschul, et al. (1997) Nucleic Acids Res. 25:3389. Alternatively, PSI-BLAST (in BLAST 2.0) can be used to perform an iterated search that detects distant relationships between molecules (Altschul, et al. (1997) supra). When utilizing BLAST, Gapped BLAST, PSI-BLAST and the default parameters of the respective programs (e.g., BLASTN for nucleotide sequences, BLASTX for proteins) can be used (the National Center for Biotechnology Information of the National Library of Medicine of the National Institutes of Health of the U.S. government). Alignment may also be performed by manual inspection.

Paired sequence identity/similarity values can be obtained using GAP Version 10 with the following parameters: % identity and % similarity for a nucleotide sequence using GAP Weight of 50 and Length Weight of 3 and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch. (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the Quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (Henikoff and Henikoff. (1989) Proc. Natl. Acad. Sci. USA 89:10915).

Unless stated otherwise,multiple alignments of the sequences provided herein are performed using the Clustal V method of alignment (Higgins and Sharp. (1989) CABIOS. 5:151-153) with the default parameters (GAP PENALTY=10, GAP LENGTH PENALTY=10). Default parameters for pairwise alignments and calculation of percent identity of amino acid sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” is calculated by determining the number of positions at which the identical nucleic acid base or amino acid residue occurs in both sequences to yield the number of matched positions, dividing the number of matched positions by the total number of positions in the window of comparison, and multiplying the result by 100.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

Embodiments include isolated polynucleotides and polypeptides, and recombinant DNA constructs useful for conferring drought tolerance; compositions (such as plants or seeds) comprising these recombinant DNA constructs; and methods utilizing these recombinant DNA constructs.

Isolated Polynucleotides and Polypeptides:

The present disclosure includes the following isolated polynucleotides and polypeptides:

An isolated polynucleotide comprising: (i) a nucleic acid sequence encoding a polypeptide having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, to SEQ ID NO:5, 8, 11, 14, 17, 20, 23, or 29; or (ii) a full complement of the nucleic acid sequence of (i), wherein the full complement and the nucleic acid sequence of (i) consist of the same number of nucleotides and are 100% complementary. Any of the foregoing isolated polynucleotides may be utilized in any recombinant DNA constructs of the present disclosure. Over-expression of the encoded polypeptideincreases plant drought tolerance, coldtolerance and/orparaquat toleranceactivity.

An isolated polynucleotide comprising: (i) a nucleic acid sequence encoding a polypeptide having at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, to SEQ ID NO: 26; or (ii) a full complement of the nucleic acid sequence of (i), wherein the full complement and the nucleic acid sequence of (i) consist of the same number of nucleotides and are 100% complementary. Any of the foregoing isolated polynucleotides may be utilized in any suppression DNA constructsof the present disclosure. Suppressedexpression of the encoded polypeptide increases plant drought tolerance activity.

An isolated polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, to SEQ ID NO: 5, 8, 11, 14, 17, 20, 23, or 29. The polypeptide is preferably a drought tolerance polypeptide or a cold tolerance polypeptide. Over-expression of the polypeptide increases plant drought tolerance, cold toleranceand/orparaquat toleranceactivity.

An isolated polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, to SEQ ID NO: 26. The polypeptide is preferably a drought sensitive polypeptide. Suppressed expression of the polypeptide increases plant drought tolerance activity.

An isolated polynucleotide comprising (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, to SEQ ID NO:4, 7, 10, 13, 16, 19, 22, or 28;(ii) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, to SEQ ID NO: 3, 6, 9, 12, 15, 18, 21 or 27; or (iii) a full complement of the nucleic acid sequence of (i) or (ii). Any of the foregoing isolated polynucleotides may be utilized in any recombinant DNA constructs of the present disclosure. The isolated polynucleotide preferably encodesadrought tolerance polypeptide or a cold tolerance polypeptide. Over-expression of the polypeptide improves plant drought tolerance, cold tolerance and/orparaquat toleranceactivity.

An isolated polynucleotide comprising (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, to SEQ ID NO: 24 or 25; (ii) a full complement of the nucleic acid sequence of (i). Any of the foregoing isolated polynucleotides may be utilized in any suppression DNA constructsof the present disclosure. The isolated polynucleotide preferably encodesadrought sensitivepolypeptide. Suppressed expression of the polypeptide preferably improves plant drought tolerance activity.

Recombinant DNA Constructs and Suppression DNA Constructs:

In one aspect, the present disclosure includes recombinant DNA constructs (including suppression DNA constructs).

In one embodiment, a recombinant DNA construct comprises a polynucleotide operably linked to at least one regulatory sequence (e.g., a promoter functional in a plant), wherein the polynucleotide comprises (i) a nucleic acid sequence encoding an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, to SEQ ID NO: 5, 8, 11, 14, 17, 20, 23, or 29;or (ii) a full complement of the nucleic acid sequence of (i).

In another embodiment, a recombinant DNA construct comprises a polynucleotide operably linked to at least one regulatory sequence (e.g., a promoter functional in a plant), wherein said polynucleotide comprises (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, to SEQ ID NO: 4, 7, 10, 13, 16, 19, 22, or 28; (ii) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, to SEQ ID NO: 3, 6, 9, 12, 15, 18, 21 or 27;or (iii) a full complement of the nucleic acid sequence of (i) or (ii).

In another embodiment, a recombinant DNA construct comprises a polynucleotide operably linked to at least one regulatory sequence (e.g., a promoter functional in a plant), wherein said polynucleotide encodes adrought tolerance polypeptide or a cold tolerance polypeptide. The polypeptide preferably has drought tolerance, cold tolerance and/or paraquat toleranceactivity. The polypeptide may be from, for example, Oryza sativa, Arabidopsis thaliana, Zea mays, Glycine max, Glycine tabacina, Glycine scja or Glycine tomentella.

In another aspect, the present disclosure includes suppression DNA constructs.

A suppression DNA construct may comprise at least one regulatory sequence (e.g., a promoter functional in a plant) operably linked to (a) all or part of: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, to SEQ ID NO:26; or (ii) a full complement of the nucleic acid sequence of (a)(i); or (b) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a drought sensitivepolypeptide; or (c) all or part of: (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, to SEQ ID NO: 24 or 25; or (ii) a full complement of the nucleic acid sequence of (c)(i). The suppression DNA construct may comprise a cosuppression construct, antisense construct, viral-suppression construct, hairpin suppression construct, stem-loop suppression construct, double-stranded RNA-producing construct, RNAi construct, or small RNA construct (e.g., an siRNA construct or an miRNA construct).

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, include lowering, reducing, declining, decreasing, inhibiting, eliminating or preventing. “Silencing” or “gene silencing” does not specify mechanism and is inclusive of, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand) of the gene of interest.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as siRNA (short interfering RNA) constructs and miRNA (microRNA) constructs.

“Antisense inhibition” refers to the production of antisense RNA transcripts capable of suppressing the expression of the target gene or gene product. “Antisense RNA” refers to an RNA transcript that is complementary to all or part of a target primary transcript or mRNA and that blocks the expression of a target isolated nucleic acid fragment (for example, U.S. Pat. No. 5,107,065). The complementarity of an antisense RNA may be with respect to any part of the specific gene transcript, i.e., at the 5′ non-coding sequence, 3′ non-coding sequence, introns, or the coding sequence.

“Cosuppression” refers to the production of sense RNA transcripts capable of suppressing the expression of the target gene or gene product. “Sense” RNA refers to RNA transcript that includes the mRNA and can be translated into protein within a cell or in vitro. Cosuppression constructs in plants have been previously designed by focusing on over-expression of a nucleic acid sequence having homology to a native mRNA, in the sense orientation, which results in the reduction of all RNA having homology to the overexpressed sequence (Vaucheret et al. (1998) Plant J. 16:651-659; and Gura. (2000) Nature 404:804-808).

RNA interference (RNAi) refers to the process of sequence-specific post-transcriptional gene silencing (PTGS)in animals mediated by short interfering RNAs (siRNAs) (Fire et al. (1998) Nature 391:806). The corresponding process in plants is commonly referred to as PTGS or RNA silencing and is also referred to as quelling in fungi. The process of PTGSis thought to be an evolutionarily-conserved cellular defense mechanism used to prevent the expression of foreign genes and is commonly shared by diverse flora and phyla (Fire et al. (1999) Trends Genet. 15:358).

Small RNAs play an important role in controlling gene expression, forexample, small RNAs regulate many developmental processes which include flowering. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to 24 nucleotides (nt) in length that have been identified in both animals and plants (Lagos-Quintana et al. (2001) Science 294:853-858, Lagos-Quintana et al. (2002) Curr. Biol. 12:735-739; Lau et al. (2001) Science 294:858-862; Lee and Ambros. (2001) Science 294:862-864; Llave et al. (2002) Plant Cell 14:1605-1619; Mourelatos et al. (2002) Genes Dev. 16:720-728; Park et al. (2002) Curr. Biol. 12:1484-1495; Reinhart et al.(2002) Genes Dev. 16:1616-1626). They are processed from longer precursor transcripts that range in size from approximately 70 to 200 nt, and these precursor transcripts have the ability to form stable hairpin structures.

miRNAs appear to regulate target genes by binding to complementary sequences located in the transcripts produced by these genes. It seems likely that miRNAs can enter at least two pathways of target gene regulation: (1) translational inhibition; and (2) RNA cleavage. miRNAs entering the RNA cleavage pathway are analogous to the 21-25 ntsiRNAs generated during RNAi in animals and PTGS in plants, and likely are incorporated into an RNA-induced silencing complex (RISC) that is similar or identical to that seen for RNAi.

Regulatory Sequences:

A recombinant DNA construct (including a suppression DNA construct) of the present disclosure may comprise at least one regulatory sequence.

A regulatory sequence may be a promoter.

A number of promoters can be used in recombinant DNA constructs of the present disclosure. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High-level, constitutive expression of the candidate gene under control of the 35S or UBI promoter may have pleiotropic effects, although candidate gene efficacy may be estimated when driven by a constitutive promoter. Use of tissue-specific and/or stress-induced promoters may eliminate undesirable effects but retain the ability to enhance drought tolerance. This effect has been observed in Arabidopsis (Kasuga et al. (1999) Nature Biotechnol. 17:287-91).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the disclosure, it may be desirable to use a tissue-specific or developmentally regulated promoter.

A tissue-specific or developmentally-regulated promoter is a DNA sequence which regulates the expression of a DNA sequence selectively in the cells/tissues of a plant, such as in those cells/tissues critical to tassel development, seed set, or both, and which usually limits the expression of such a DNA sequence to the developmental period of interest (e.g. tassel development or seed maturation) in the plant. Any identifiable promoter which causes the desired temporal and spatial expression may be used in the methods of the present disclosure.

Many leaf-preferred promoters are known in the art (Yamamoto et al. (1997) Plant J. 12(2):255-265; Kwon et al. (1994) Plant Physiol. 105:357-367; Yamamoto et al. (1994) Plant Cell Physiol. 35(5):773-778; Gotoret al. (1993) Plant J. 3:509-518; Orozco et al. (1993) Plant Mol. Biol. 23(6):1129-1138; and Matsuoka et al. (1993) Proc. Natl. Acad. Sci. USA 90(20):9586-9590).

Promoters which are seed or embryo-specific and may be useful in the disclosure include soybean Kunitz trypsin inhibitor (Kti3, Jofuku and Goldberg. (1989) Plant Cell 1:1079-1093), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564). Promoters of seed-specific genes operably linked to heterologous coding regions in chimeric gene constructions maintain their temporal and spatial expression pattern in transgenic plants. Such examples include Arabidopsis 2S seed storage protein gene promoter to express enkephalin peptides in Arabidopsis and Brassica napus seeds (Vanderkerckhove et al. (1989) Bio/Technology 7:L929-932), bean lectin and bean beta-phaseolin promoters to express luciferase (Riggs et al. (1989) Plant Sci. 63:47-57), and wheat glutenin promoters to express chloramphenicol acetyl transferase (Colot et al. (1987) EMBO J 6:3559-3564).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters for use in certain embodiments include the following: 1) the stress-inducible promoter RD29A (Kasuga et al. (1999) Nature Biotechnol. 17:287-291); 2) the stress-inducible promoter Rab17 (Vilardell et al. (1991) Plant Mol. Bio. 17:985-993; Kamp Busk et al. (1997) Plant J 11(6):1285-1295); 3) the barley promoter B22E whose expression is specific to the pedicel in developing maize kernels (“Primary Structure of a Novel Barley Gene Differentially Expressed in Immature Aleurone Layers”. Klemsdal, S. S. et al. (1991) Mol. Gen. Genet. 228(1/2):9-16); and 4) maize promoter Zag2 (“Identification and molecular characterization of ZAG1, the maize homolog of the Arabidopsis floral homeotic gene AGAMOUS”, Schmidt, R. J. et al. (1993) Plant Cell 5(7):729-737; “Structural characterization, chromosomal localization and phylogenetic evaluation of two pairs of AGAMOUS-like MADS-box genes from maize”, Theissen et al. (1995) Gene 156(2):155-166; NCBI GenBank Accession No. X80206)). Zag2 transcripts can be detected 5 days prior to pollination to 7 to 8 days after pollination (“DAP”), and directs expression in the carpel of developing female inflorescences and CimI which is specific to the nucleus of developing maize kernels. CimI transcript is detected 4 to 5 days before pollination to 6 to 8 DAP. Other useful promoters include any promoter which can be derived from a gene whose expression is maternally associated with developing female florets.

For the expression of a polynucleotide in developing seed tissue, promoters of particular interest include seed-preferred promoters, particularly early kernel/embryo promoters and late kernel/embryo promoters. Kernel development post-pollination is divided into approximately three primary phases. The lag phase of kernel growth occurs from about 0 to 10-12 DAP. During this phase the kernel is not growing significantly in mass, but rather important events are being carried out that will determine kernel vitality (e.g., number of cells established). The linear grain fill stage begins at about 10-12 DAP and continues to about 40 DAP. During this stage of kernel development, the kernel attains almost all of its final mass, and various storage products (i.e., starch, protein, oil) are produced. Finally, the maturation phase occurs from about 40 DAP to harvest. During this phase of kernel development the kernel becomes quiescent and begins to dry down in preparation for a long period of dormancy prior to germination. As defined herein “early kernel/embryo promoters” are promoters that drive expression principally in developing seed during the lag phase of development (i.e., from about 0 to about 12 DAP). “Late kernel/embryo promoters”, as defined herein, drive expression principally in developing seed from about 12 DAP through maturation. There may be some overlap in the window of expression. The choice of the promoter will depend on the ABA-associated sequence utilized and the phenotype desired. p Early kernel/embryo promoters include, for example, Cim1 that is active 5 DAP in particular tissues (WO 00/11177), which is herein incorporated by reference. Other early kernel/embryo promoters include the seed-preferred promoters end1 which is active 7-10 DAP, and end2, which is active 9-14 DAP in the whole kernel and active 10 DAP in the endosperm and pericarp (WO 00/12733), herein incorporated by reference. Additional early kernel/embryo promoters that find use in certain methods of the present disclosure include the seed-preferred promoter Itp2 (U.S. Pat. No. 5,525,716); maize Zm40 promoter (U.S. Pat. No. 6,403,862); maize nuc1c (U.S. Pat. No. 6,407,315); maize ckx1-2 promoter (U.S. Pat. No. 6,921,815 and US Patent Application Publication Number 2006/0037103); maize led promoter (U.S. Pat. No. 7,122,658); maize ESR promoter (U.S. Pat. No. 7,276,596); maize ZAP promoter (U.S. Patent Application Publication Numbers 20040025206 and 20070136891); maize promoter eep1 (U.S. Patent Application Publication Number 20070169226); and maize promoter ADF4 (U.S. Patent Application No. 60/963,878, filed 7 Aug. 2007).

Additional promoters for regulating the expression of the nucleotide sequences of the present disclosure in plants are stalk-specific promoters, including the alfalfa S2A promoter (GenBank Accession No. EF030816; Abrahams et al. (1995) Plant Mol. Biol. 27:513-528) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

Promoters for use in certain embodiments of the current disclosure may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays; root preferred promoters, such as the maize NAS2 promoter, the maize Cyclo promoter (US 2006/0156439, published Jul. 13, 2006), the maize ROOTMET2 promoter (WO05063998, published Jul. 14, 2005), the CR1BIO promoter (WO06055487, published May 26, 2006), the CRWAQ81 (WO05035770, published Apr. 21, 2005) and the maize ZRP2.47 promoter (NCBI accession number: U38790; GI No. 1063664).

Recombinant DNA constructs of the present disclosure may also include other regulatory sequences, including but not limited to, translation leader sequences, introns, and polyadenylation recognition sequences. In certain embodiments, a recombinant DNA construct further comprises an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg. (1988) Mol. Cell Biol. 8:4395-4405; Callis et al. (1987) Genes Dev. 1:1183-1200).

Any plant can be selected for the identification of regulatory sequences and polypeptide genes to be used in recombinant DNA constructs of the present disclosure. Examples of suitable plant targets for the isolation of genes and regulatory sequences would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, mango, melon, mushroom, nectarine, nut, oat, oil palm, oil seed rape, okra, olive, onion, orange,ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, switchgrass, tangerine, tea, tobacco, tomato, triticale, turf, turnip, vine, watermelon, wheat, yams, and zucchini.

Compositions:

A composition of the present disclosure is a plant comprising in its genome any of the recombinant DNA constructs (including any of the suppression DNA constructs) of the present disclosure (such as any of the constructs discussed above). Compositions also include any progeny of the plant, and any seed obtained from the plant or its progeny, wherein the progeny or seed comprises within its genome the recombinant DNA construct (or suppression DNA construct). Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced recombinant DNA construct (or suppression DNA construct). These seeds can be grown to produce plants that would exhibit an altered agronomic characteristics (e.g., an increased agronomic characteristicsoptionally under water limiting conditions), or used in a breeding program to produce hybrid seed, which can be grown to produce plants that would exhibit such an altered agronomic characteristics. The seeds may be maize seeds or rice seeds.

The plant may be a monocotyledonous or dicotyledonous plant, for example, a rice or maize or soybean plant, such as a maize hybrid plant or a maize inbred plant. The plant may also be sunflower, sorghum, canola, wheat, alfalfa, cotton, barley, millet, sugar cane or switchgrass.

The recombinant DNA construct may be stably integrated into the genome of the plant.

Particular embodiments include but are not limited to the following:

1. A transgenic plant (for example, a rice ormaize or soybeanpiant) comprising in its genome a recombinant DNA construct comprising a polynucleotide operably linked to at least one regulatory sequence, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, to SEQ ID NO: 5, 8, 11, 14, 17, 20, 23, or 29, and wherein said plant exhibits increased drought tolerance, cold tolerance and/or paraquat tolerancewhen compared to a control plant. The plant may further exhibit an alteration of at least one agronomic characteristics when compared to the control plant.

2. A transgenic plant (for example, a rice or maize or soybean plant) comprising in its genome a recombinant DNA construct comprising a polynucleotide operably linked to at least one regulatory sequence, wherein said polynucleotide encodes a polypeptide, and wherein said plant exhibits increased drought tolerance, cold tolerance and/or paraquat tolerancewhen compared to a control plant. The plant may further exhibit an alteration of at least one agronomic characteristics when compared to the control plant.

3. A transgenic plant (for example, a rice or maize or soybean plant) comprising in its genome a recombinant DNA construct comprising a polynucleotide operably linked to at least one regulatory sequence, wherein said polynucleotide encodes a polypeptide, and wherein said plant exhibits an alteration of at least one agronomic characteristics when compared to a control plant.

4. A transgenic plant (for example, a rice or maize or soybean plant) comprising in its genome a suppression DNA construct comprising at least one regulatory element operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%,85%, 86%, 87%, 88%, 89%, 90%, 91%, 92%, 93%, 94%, 95%, 96%, 97%, 98%, 99%, or 100% sequence identity, based on the Clustal V method of alignment, to said all or part of a sense strand or antisense strand from which said region is derived, and wherein said target gene of interest encodes a drought sensitive polypeptide, and wherein said plant exhibits an alteration of at least one agronomic characteristics when compared to a control plant.

5. A transgenic plant (for example, a rice or maize or soybean plant) comprising in its genome a suppression DNA construct comprising at least one regulatory element operably linked to all or part of (a) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, to SEQ ID NO: 26; or (b) a full complement of the nucleic acid sequence of (a), and wherein said plant exhibits an alteration of at least one agronomic characteristics when compared to a control plant.

6. Any progeny of the above plants in embodiment 1-5, any seeds of the above plants in embodiment 1-5, any seeds of progeny of the above plants in embodiment 1-5, and cells from any of the above plants in embodiment 1-5 and progeny thereof.

In any of the foregoing embodiment 1-6 or other embodiments, the drought tolerance polypeptide or cold tolerance polypeptide may be from Oryza sativa, Oryza australiensis, Oryzabarthii, Oryza glaberrima (African rice), Oryza latifolia, Oryza longistaminata, Oryza meridionalis, Oryza officinalis, Oryza punctata, Oryza rufipogon (brownbeard or red rice), Oryza nivara (Indian wild rice), Arabidopsis thaliana, Zea mays, Glycine max, Glycine tabacina, Glycine scja or Glycine tomentella.

In any of the foregoing embodiment 1-6 or other embodiments, the recombinant DNA construct (or suppression DNA construct) may comprise at least a promoter functional in a plant as a regulatory sequence.

In any of the foregoing embodiment 1-6 or other embodiments, the alteration of at least one agronomic characteristics is either an increase or decrease.

In any of the foregoing embodiment 1-6 or other embodiments, the at least one agronomic characteristics may be selected from the group consisting of greenness, grain yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, tiller number, panicle size, early seedling vigor and seedling emergence under low temperature stress. For example, the alteration of at least one agronomic characteristic may be an increase in grain yield, greenness or biomass.

In any of the foregoing embodiment 1-6 or other embodiments, the plant may exhibit the alteration of at least one agronomic characteristics when compared, under water limiting conditions, to a control plant.

In any of the foregoing embodiment 1-6 or other embodiments, the plant may exhibit the alteration of at least one agronomic characteristics when compared, under low temperature conditions, to a control plant.

In any of the foregoing embodiment 1-6 or other embodiments, the plant may exhibit the alteration of at least one agronomic characteristics when compared, under oxidative stress (paraquat) conditions, to a control plant.

“Drought” refers to a decrease in water availability to a plant that, especially when prolonged or when occurring during critical growth periods, can cause damage to the plant or prevent its successful growth (e.g., limiting plant growth or seed yield).

“Drought tolerance” reflects a plant's ability to survive under drought without exhibiting substantial physiological or physical deterioration, and/or its ability to recover when water is restored following a period of drought.

“Drought tolerance activity” of a polypeptide indicates that over-expression of the polypeptide in a transgenic plant confers increased drought tolerance of the transgenic plant relative to a reference or control plant.

“Increased drought tolerance” of a plant is measured relative to a reference or control plant, and reflects ability of the plant to survive under drought conditions with less physiological or physical deterioration than a reference or control plant grown under similar drought conditions, or ability of the plant to recover more substantially and/or more quickly than would a control plant when water is restored following a period of drought.

“Environmental conditions” refer to conditions under which the plant is grown, such as the availability of water, availability of nutrients, or the presence of insects or disease.

“Paraquat” (1,1-dimethyl-4,4-bipyridinium dichloride), is a foliar-applied and non-selective bipyridinium herbicides, and causes photooxidative stress which further cause damage to plant or prevent its successful growth.

“Paraquat tolerance” is a trait of a plant, reflects the ability to survive and/or grow better when treated with Paraquat solution, compared to a reference or control plant.

“Increased paraquat tolerance” of a plant is measured relative to a reference or control plant, and reflects ability of the plant to survive with less physiological or physical deterioration than a reference or control plant after treated with paraquat solution. In general, tolerance to relative low level of paraquat can be used as a marker of abiotic stress tolerance, such as drought tolerance.

“Oxidative stress” reflects an imbalance between the systemic manifestation of reactive oxygen species and a biological system's ability to readily detoxify the reactive intermediates or to repair the resulting damage. Disturbances in the normal redoxstate of cells can cause toxic effects through the production of peroxides and free radicals that damage all components of the cell, including proteins, lipids, and DNA.

The Examples below describe some representative protocols and techniques for simulating drought conditions and/or evaluating drought tolerance; simulating oxidative conditions; and simulating low temperature conditions.

One can also evaluate drought tolerance by the ability of a plant to maintain sufficient yield (at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% yield) in field testing under simulated or naturally-occurring drought conditions (e.g., by measuring for substantially equivalent yield under drought conditions compared to non-drought conditions, or by measuring for less yield loss under drought conditions compared to yield loss exhibited by a control or reference plant).

Parameters such as recovery degree, survival rate, paraquat tolerance rate, gene expression level, water use efficiency, level or activity of an encoded protein, and others are typically presented with reference to a control cell or control plant. A “control” or “control plant” or “control plant cell” provides a reference point for measuring changes in phenotype of a subject plant or plant cell in which genetic alteration, such as transformation, has been effected as to a gene of interest. A subject plant or plant cell may be descended from a plant or cell so altered and will comprise the alteration. One of ordinary skill in the art would readily recognize a suitable control or reference plant to be utilized when assessing or measuring an agronomic characteristics or phenotype of a transgenic plant using compositions or methods as described herein. For example, by way of non-limiting illustrations:

1. Progeny of a transformed plant which is hemizygous with respect to a recombinant DNA construct (or suppression DNA construct), such that the progeny are segregating into plants either comprising or not comprising the recombinant DNA construct (or suppression DNA construct): the progeny comprising the recombinant DNA construct (or suppression DNA construct) would be typically measured relative to the progeny not comprising the recombinant DNA construct (or suppression DNA construct). The progeny not comprising the recombinant DNA construct (or the suppression DNA construct) is the control or reference plant.

2. Introgression of a recombinant DNA construct (or suppression DNA construct) into an inbred line, such as in rice and maize, or into a variety, such as in soybean: the introgressed line would typically be measured relative to the parent inbred or variety line (i.e., the parent inbred or variety line is the control or reference plant).

3. Two hybrid lines, wherein the first hybrid line is produced from two parent inbred lines, and the second hybrid line is produced from the same two parent inbred lines except that one of the parent inbred lines contains a recombinant DNA construct (or suppression DNA construct): the second hybrid line would typically be measured relative to the first hybrid line (i.e., the first hybrid line is the control or reference plant).

4. A plant comprising a recombinant DNA construct (or suppression DNA construct): the plant may be assessed or measured relative to a control plant not comprising the recombinant DNA construct (or suppression DNA construct) but otherwise having a comparable genetic background to the plant (e.g., sharing at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%,85%, 86%, 87%, 88%, 89%, 90%, 91%, 92%, 93%, 94%, 95%, 96%, 97%, 98%, 99%, or 100% sequence identity of nuclear genetic material compared to the plant comprising the recombinant DNA construct (or suppression DNA construct)). There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s), and Simple Sequence Repeats (SSRs) which are also referred to as Microsatellites.

A control plant or plant cell may comprise, for example: (a) a wild-type (WT) plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimulus that would induce expression of the gene of interest or (e) the subject plant or plant cell itself, under conditions in which the gene of interest is not expressed. A control may comprise numerous individuals representing one or more of the categories above; for example, a collection of the non-transformed segregants of category “c” is often referred to as a bulk null.

In this disclosure, EN, ZH11-TC, and VC indicate control plants, ENrepresents event null segregated from the transgenic rice plant, ZH11-TC represents rice plants generated from tissue cultured Zhonghua11, and VC represents plants transformed with empty vector of DP0005or DP0158.

Methods:

Methods include but are not limited to methods for increasing drought tolerance in a plant, methods for evaluating drought tolerance in a plant, methods for increasing cold tolerance in a plant, methods for increasing paraquat tolerance, methods for altering an agronomic characteristics in a plant, methods for determining an alteration of an agronomic characteristics in a plant, and methods for producing seed. The plant may be a monocotyledonous or dicotyledonous plant, for example, rice, maize or soybean plant. The plant may also be sunflower, canola, wheat, alfalfa, cotton, barley, millet, sugar cane or sorghum. The seed may be a maize or soybean seed, for example, a maize hybrid seed or maize inbred seed.

Methods include but are not limited to the following:

A method for transforming a cell comprising transforming a cell with any one or more of the isolated polynucleotides of the present disclosure, wherein, in particular embodiments, the cell is eukaryotic cell, e.g., a yeast, insect or plant cell; or prokaryotic cell, e.g., a bacterial cell.

A method for producing a transgenic plant comprising transforming a plant cell with any of the isolated polynucleotides or recombinant DNA constructs (including suppression DNA constructs) of the present disclosure and regenerating a transgenic plant from the transformed plant cell, wherein, the transgenic plant and the transgenic seed obtained by this method may be used in other methods of the present disclosure.

A method for isolating a polypeptide of the disclosure from a cell or culture medium of the cell, wherein the cell comprises a recombinant DNA construct comprising a polynucleotide of the disclosure operably linked to at least one regulatory sequence, and wherein the transformed host cell is grown under conditions that are suitable for expression of the recombinant DNA construct.

A method for altering the level of expression of a polypeptide of the disclosure in a host cell comprising: (a) transforming a host cell with a recombinant DNA construct of the present disclosure; and (b) growing the transformed host cell under conditions that are suitable for the expression of the recombinant DNA construct, wherein the expression of the recombinant DNA construct results in production of altered levels of the polypeptide of the disclosure in the transformed host cell.

A method of increasing drought tolerance, cold tolerance and/or paraquat tolerance in a plant, comprising: (a) introducing into a regenerable plant cell a recombinant DNA construct comprising a polynucleotide operably linked to at least one regulatory sequence (for example, a promoter functional in a plant), wherein the polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, to SEQ ID NO: 5, 8, 11, 14, 17, 20, 23 or 29; (b) regenerating a transgenic plant from the regenerable plant cell after step (a), wherein the transgenic plant comprises in its genome the recombinant DNA construct andexhibits increased drought tolerance, cold tolerance and/or paraquat tolerance when compared to a control plant; and further (c) obtaining a progeny plant derived from transgenic plant, wherein said progeny plant comprises in its genome the recombinant DNA construct and exhibits increased drought tolerance, cold tolerance and/or paraquat tolerancewhen compared to a control plant.

A method of evaluating drought tolerance, cold tolerance and/or paraquat tolerancein a plant comprising (a) obtaining a transgenic plant, which comprises in its genome a recombinant DNA construct comprising a polynucleotide operably linked to at least one regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%,90%, 91%, 92%, 93%, 94%, 95%, 96%, 97%, 98%, 99%, or 100% sequence identity, based on the Clustal V method of alignment, to SEQ ID NO:5, 8, 11, 14, 17, 20, 23 or 29; (b) obtaining a progeny plant derived from said transgenic plant, wherein the progeny plant comprises in its genome the recombinant DNA construct; and (c) evaluating the progeny plant for drought tolerance, cold tolerance and/or paraquat tolerance compared to a control plant.

A method of evaluating drought tolerancein a plant comprising (a) obtaining a transgenic plant, wherein the transgenic plant comprises in its genome a suppression DNA construct comprising at least one regulatory sequence (for example, a promoter functional in a plant) operably linked to all or part of (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:26, or (ii) a full complement of the nucleic acid sequence of (a)(i); (b) obtaining a progeny plant derived from said transgenic plant, wherein the progeny plant comprises in its genome the suppression DNA construct; and (c) evaluating the progeny plant for drought tolerancecompared to a control plant.

A method of evaluating drought tolerancein a plantcomprising (a) obtaining a transgenic plant, wherein the transgenic plant comprises in its genome a suppression DNA construct comprising at least one regulatory sequence (for example, a promoter functional in a plant) operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a polypeptide; (b) obtaining a progeny plant derived from the transgenic plant, wherein the progeny plant comprises in its genome the suppression DNA construct; and (c) evaluating the progeny plant for drought tolerancecompared to a control plant.

A method of determining an alteration of an agronomic characteristics in a plantcomprising (a) obtaining a transgenic plant which comprises in its genome a recombinant DNA construct comprising a polynucleotide operably linked to at least one regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:5, 8, 11, 14, 17, 20, 23, 26 or 29; (b) obtaining a progeny plant derived from said transgenic plant, wherein the progeny plant comprises in its genome the recombinant DNA construct; and (c) determining whether the progeny plant exhibits an alteration in at least one agronomic characteristics when compared, optionally under water limiting conditions and/or cold stress, to a control plant.

A method of producing seed comprising any of the preceding methods, and further comprising obtaining seeds from said progeny plant, wherein said seeds comprise in their genome said recombinant DNA construct (or suppression DNA construct).

In any of the preceding methods or any other embodiments of methods of the present disclosure, in said introducing step,the said regenerable plant cell may comprise a callus cell, an embryogenic callus cell, a gametic cell, a meristematic cell, or a cell of an immature embryo. The regenerable plant cells may derive from an inbred maize plant.

In any of the preceding methods or any other embodiments of methods of the present disclosure, said regenerating step may comprise the following: (i) culturing said transformed plant cells in a medium comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other embodiments of methods of the present disclosure, the step of determining an alteration of an agronomic characteristics in a transgenic plant, if applicable, may comprise determining whether the transgenic plant exhibits an alteration of at least one agronomic characteristics when compared, under varying environmental conditions, to a control plant not comprising the recombinant DNA construct.

In any of the preceding methods or any other embodiments of methods of the present disclosure, the step of determining an alteration of an agronomic characteristics in a progeny plant, if applicable, may comprise determining whether the progeny plant exhibits an alteration of at least one agronomic characteristics when compared, under varying environmental conditions, to a control plant not comprising the recombinant DNA construct.

In any of the preceding methods or any other embodiments of methods of the present disclosure, the plant may exhibit the alteration of at least one agronomic characteristics when compared, under water limiting conditions and/or cold stress conditions, to a control plant.

In any of the preceding methods or any other embodiments of methods of the present disclosure, alternatives exist for introducing into a regenerable plant cell a recombinant DNA construct comprising a polynucleotide operably linked to at least one regulatory sequence. For example, one may introduce into a regenerable plant cell a regulatory sequence (such as one or more enhancers, optionally as part of a transposable element), and then screen for an event in which the regulatory sequence is operably linked to an endogenous gene encoding a polypeptide of the instant disclosure.

The introduction of recombinant DNA constructs of the present disclosure into plants may be carried out by any suitable technique, including but not limited to direct DNA uptake, chemical treatment, electroporation, microinjection, cell fusion, infection, vector-mediated DNA transfer, bombardment, or Agrobacterium-mediated transformation. Techniques for plant transformation and regeneration have been described in International Patent Publication WO 2009/006276, the contents of which are herein incorporated by reference.

In addition, methods to modify or alter the host endogenous genomic DNA are available. This includes altering the host native DNA sequence or a pre-existing transgenic sequence including regulatory elements, coding and non-coding sequences. These methods are also useful in targeting nucleic acids to pre-engineered target recognition sequences in the genome. As an example, the genetically modified cell or plant described herein, is generated using “custom”engineered endonucleases such asmeganucleases produced to modify plant genomes (e.g., WO 2009/114321; Gao et al. (2010) Plant Journal 1:176-187). Another site-directed engineering is through the use of zinc finger domain recognition coupled with the restriction properties of restriction enzyme (e.g., Urnov, et al. (2010) Nat Rev Genet. 11(9):636-46; Shukla, et al. (2009) Nature 459 (7245):437-41). A transcription activator-like (TAL) effector-DNA modifying enzyme (TALE or TALEN) is also used to engineer changes in plant genome. See e.g., US20110145940, Cermak et al., (2011) Nucleic Acids Res. 39(12) and Boch et al., (2009), Science 326(5959): 1509-12. Site-specific modification of plant genomes can also be performed using the bacterial type II CRISPR (clustered regularly interspaced short palindromic repeats)/Cas (CRISPR-associated) system. See e.g., Belhaj et al., (2013), Plant Methods 9: 39; The CRISPR/Cas system allows targeted cleavage of genomic DNA guided by a customizable small noncoding RNA.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant containing a desired polypeptide is cultivated using methods well known to one skilled in the art.

### EXAMPLES

The present disclosure is further illustrated in the following examples, in which parts and percentages are by weight and degrees are Celsius, unless otherwise stated. It should be understood that these examples, while indicating embodiments of the disclosure, are given by way of illustration only. From the above discussion and these examples, one skilled in the art can ascertain the essential characteristics of this disclosure, and without departing from the spirit and scope thereof, can make various changes and modifications of the disclosure to adapt it to various usages and conditions. Furthermore, various modifications of the disclosure in addition to those shown and described herein will be apparent to those skilled in the art from the foregoing description. Such modifications are also intended to fall within the scope of the appended claims.

### Example 1

### Drought Tolerance Genesand ColdTolerance Genes Cloning and Over-Expression Vector Construction

Based on our preliminary screening of rice activation tagging population and the sequence information of gene IDsshown in Table 2, primers were designed for cloning rice drought tolerancegenes OsDN-DTP2, OsMRP10, OsGSTU35, OsCML1, OsIMPA1a, OsMYB125and OsCML3, drought sensitive gene OsBCS1L, and cold tolerance gene OsDN-CTP1. The primers and the expected-lengths of the amplified genes are shown in Table 3.

ForOsGSTU35, OsCML1, OsIMPA1a, OsMYB125, OsCML3 and OsBCS1L, their cDNAs were from cloned pooled cDNA from leaf, stem and root tissues of Zhonghua 11 plant as the template. For OsDN-DTP2, OsMRP10 and OsDN-CTP1, theirgDNAswere cloned, and amplified using genomic DNA of Zhonghua 11 as the template. The PCR reaction mixtures and PCR procedures are shown in Table 4 and Table 5.

TABLE 2
Rice gene names, Gene IDs (from TIGR) and Construct IDs

| Gene name | LOC ID | Construct ID |
|---|---|---|
| OsDN-DTP2 | Os08g0552300 | DP0008 |
| OsMRP10 | LOC_Os04g13220 | DP0014 |
| OsGSTU35 | LOC_Os01g72130 | DP0055 |
| OsCML1 | LOC_Os01g72080 | DP0060 |
| OsIMFA1a | LOC_Os05g06350 | DP0062 |
| OsMYB125 | LOC_Os05g41240 | DP0067 |
| OsCML3 | LOC_Os12g03816 | DP0162 |
| OsBCS1L | LOC_Os05g51130 | DP0196 |
| OsDN-CTP1 | LOC_Os02g20150 | DP0142 |

TABLE 3
Primers for cloning rice drought
tolerance genes and cold tolerance gene

| | | | | Length of |
|---|---|---|---|---|
| | | SEQ | | amplified |
| | | ID | Gene | fragment |
| Primer | Sequence | NO: | name | (bp) |
| 8-Os08g0552300 | 5′-CATGGATCCGATTCAAC | 30 | OsDN-DTP2 | 2767 |
| up3 | ACAAAGAGGCAAC-3′ | | | |
| 8-Os0890552300 | 5′-ACACTCGAGGTATTTGT | 31 | | |
| down3 | CTGCAATCCTCATGTCTA | | | |
| | G-3′ | | | |
| 45-Os0490209300 | 5′-CCGCCTGCAGGCGACAC | 32 | OsMRP10 | 8471 |
| down2 | TGAGACCGAGTCGACATG | | | |
| | G-3′ | | | |
| 45-Os0490209300 | 5′-ATTCCTGCAGGATTACC | 33 | | |
| up3 | AAATTGGAATGTCAGAGAAC | | | |
| | GAG-3′ | | | |
| DEgc-356 | 5′-ACGATGGGTGAAAGGGT | 34 | OsGSTU35 | 757 |
| | GAAGCTC-3′ | | | |
| DEgc-357 | 5′-GAATCAAATAGTAACTT | 35 | | |
| | ATTCCATTCCCATG-3′ | | | |
| DEgc-336 | 5′-TCTCCCATTCGAGCGAG | 36 | OsCML1 | 647 |
| | ATGAAGC-3′ | | | |
| DEgc-337 | 5′-GAACGGAGGAATGGATC | 37 | | |
| | ACCACGATC-3′ | | | |
| DEgc-456 | 5′-GCACGAGGCTGGGGATG | 38 | OsIMPA1a | 751 |
| | ACATG-3′ | | | |
| DEgc-457 | 5′-CAACCAAGACTCCAACG | 39 | | |
| | ACAAGACTC-3′ | | | |
| DEgc-561 | 5′-ATGATGTACCATGCAAA | 40 | OsMYB125 | 837 |
| | GAAGTTCTCTGTACCCTTTG | | | |
| | GACCGCAG-3′ | | | |
| DEgc-562 | 5′-CGATCGGCCCGCAGTGG | 41 | | |
| | AGGTTAAC-3′ | | | |
| gc-2068 | 5′-CTTGTGTTACTAATAAT | 42 | OsCML3 | 686 |
| | CTTTGAGGGGAGGC-3′ | | | |
| gc-2069 | 5′-CCAGAACAAGTGTAACC | 43 | | |
| | AGAAATTGAGG-3′ | | | |
| gc-2208 | 5′-CTCACCCTCCCCATTCA | 44 | OsBCs1L | 1592 |
| | ACACTACTG-3′ | | | |
| gc-2209 | 5′-CATTCTTGTTGTCATTG | 45 | | |
| | TTGTACTCCAC-3 | | | |
| gc-1403 | 5′-CGATTTTGTCCTACATG | 46 | OsDN-CTP1 | 813 |
| | GCGGTTGAG-3′ | | | |
| gc-1404 | 5′-CGAGTTCTTGTTAATGG | 47 | | |
| | CGATGGATCACTTG-3′ | | | |

TABLE 4
PCR reaction mixture for cloning drought tolerancegenes and cold
tolerance genes

| | |
|---|---|
| Reaction mix | 50 μL |
| Template | 1 μL |
| TOYOBO KOD-FX (1.0 U/μL) | 1 μL |
| 2 × PCR buffer for KOD-FX | 25 μL |
| 2 mMdNTPs (0.4 mM each) | 10 μL |
| Primer-F/R (10 μM) | 2 μL each |
| ddH₂O | 9 μL |

TABLE 5
PCR cycle conditions

| 94° C. | 3 | min | | |
|---|---|---|---|---|
| 98° C. | 10 | s | | |
| 58° C. | 30 | s | {close oversize brace} | ×30 |
| 68° C. | (1 Kb/min) 1 min | | | |
| 68° C. | 5 | min | | |

The PCR amplified products were extracted after the agarose gel electrophoresis using a column kit and then ligated with TA cloning vectors. The sequences and orientation in these constructs were confirmed by sequencing. Then these genes were cloned into plant binary construct DP0005 (pCAMBIA1300-AsRed) (SEQ ID NO: 1) or DP0158 which was generated by transferringDsRed gene expression cassette (SEQ ID NO: 2 in the sequence list) into construct DP0005.

OsDN-DTP2, OsMRP10, OsGSTU35, OsCML1, OsIMPA1a and OsMYB125 were cloned into construct of DP0005. The generated over-expression vectors were listed in Table 2. The cloned nucleotide sequence in construct of DP0008 and coding sequence of OsDN-DTP2 are provided as SEQ ID NO: 3 and 4, the encoded amino acid sequence of OsDN-DTP2 is SEQ ID NO: 5; the cloned nucleotide sequence in construct of DP0014 and coding sequence of OsMRP10 are provided as SEQ ID NO: 6 and 7, the encoded amino acid sequence of OsMRP10 is SEQ ID NO: 8; the cloned nucleotide sequence in construct of DP0055 and coding sequence of OsGSTU35 are provided as SEQ ID NO: 9 and 10, the encoded amino acid sequence of OsGSTU35 is SEQ ID NO: 11; the cloned nucleotide sequence in construct of DP0060 and coding sequence of OsCML1 are provided as SEQ ID NO: 12 and 13, the encoded amino acid sequence of OsCML1 is SEQ ID NO: 14; the cloned nucleotide sequence in construct of DP0062 and coding sequence of OsIMPA1a are provided as SEQ ID NO: 15 and 16, the encoded amino acid sequence of OsIMPA1a is SEQ ID NO: 17; and the cloned nucleotide sequence in construct of DP0067and coding sequence of OsMYB125 are provided as SEQ ID NO: 18 and 19, the encoded amino acid sequence of OsMYB125 is SEQ ID NO: 20.

OsCML3, OsBCS1L and OsDN-CTP1 were cloned into construct of DP0158. The cloned nucleotide sequence in construct of DP0162 and coding sequence of OsCML3 are provided as SEQ ID NO: 21 and 22, the encoded amino acid sequence of OsCML3 is SEQ ID NO: 23; the cloned nucleotide sequence in construct of DP0196 and coding sequence of OsBCS1L are provided as SEQ ID NO: 24 and 25, the encoded amino acid sequence of OsBCS1L is SEQ ID NO: 26; and the cloned nucleotide sequence in construct of DP0142 and coding sequence of OsDN-CTPlare provided as SEQ ID NO: 27 and 28, the encoded amino acid sequence of OsDN-CTP1 is SEQ ID NO: 29.

### Example 2

### Transformation to Get Transgenic Rice Events

In this research, all of the over-expression vectors and empty vector (DP0005 and DP0158) were transformed into the Zhonghua 11 (Oryza sativa L.) by Agrobacteria-mediated method as described by Lin and Zhang ((2005) Plant Cell Rep. 23:540-547). Zhonghua 11 was cultivated by the Institute of Crop Sciences, Chinese Academy of Agricultural Sciences. The first batch of seeds used in this research was provided by Beijing WeimingKaituo Agriculture Biotech Co., Ltd. Calli induced from embryos was transformed with Agrobacteria with the vector. The transgenic seedlings (T₀) generated in transformation laboratory are transplanted in the field to get T₁ seeds. The T₁ and T₂ seeds are stored at cold room (4° C.), and T₂ seeds were used for following trait screening.

OsDN-DTP2, OsMRP10, OsGSTU35, OsCML1, OsIMPA1 aand OsMYB125-transgenic seeds did not show red color under green fluorescent light. The T₁ transgenic plants were selected by hygromycin by culturing the rice plants (from 1-2 cm in height) in 50 mg/L hygromycin solution, the survived plants (hygromycin-resistant) were planted in field to produce T₂ seeds. Only the hygromycin-resistant T₂ transgenic rice plants were used in trait screen.

OsCML3, OsBCS1L and OsDN-CTP1-transgenic seeds which showed red color under green fluorescent light (transgenic seeds) were used in the following assays.

### Example 3

### Gene Expression Analysis

Transgene expression levels of the genes in the transgenic rice plants were analyzed. A standard RT-PCR or a real-time RT-PCR procedure, such as the QuantiTect® Reverse Transcription Kit from Qiagen® and Real Time-PCR(SYBR® Premix Ex Tag™, TaKaRa), was used. EF-1α gene was used as an internal control to show that the amplification and loading of samples from the transgenic rice and wild-type were similar. Gene expression was normalized based on the EF-1α mRNA levels.

The primers for real-time RT-PCR for the OsBCS1L gene are listed below:

DP0196-F1:
(SEQ ID NO: 48)
5′-CCTTGGTCTACTGGAGCTCC-3′
DP0196-R1:
(SEQ ID NO: 49)
5′-GTTCTCCATCGCTTTGCTATC-3′

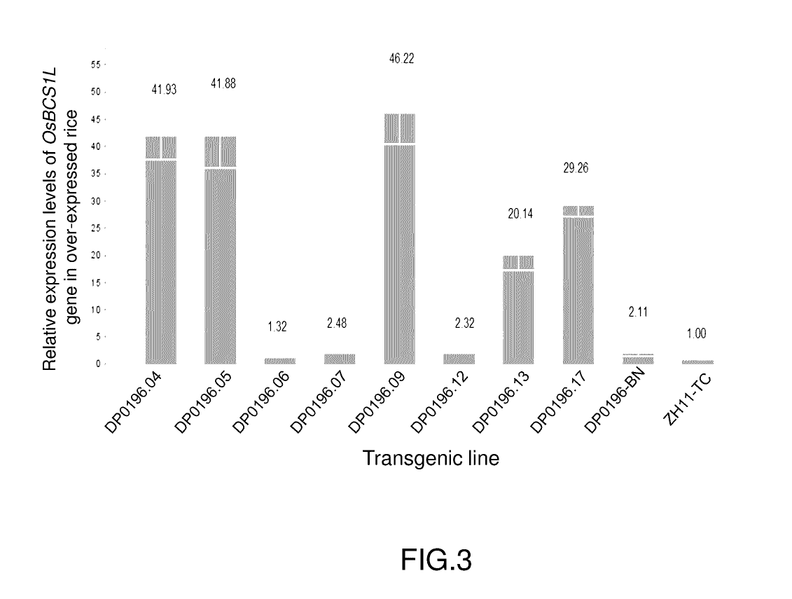

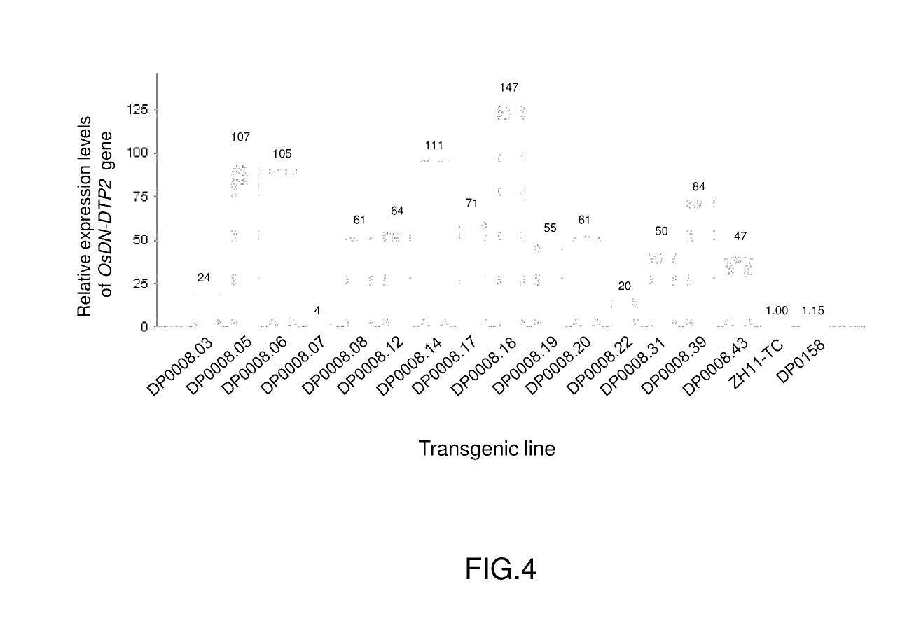

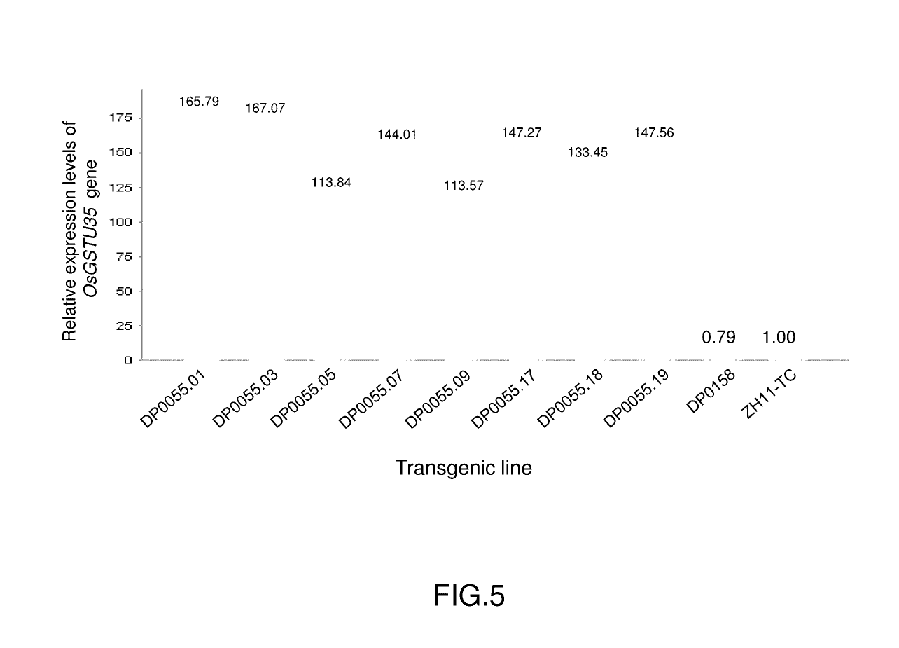

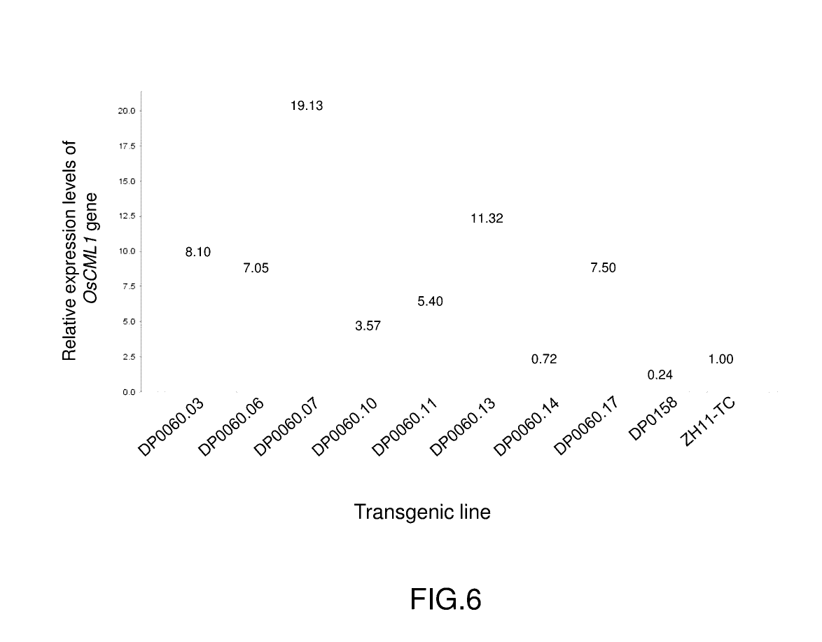

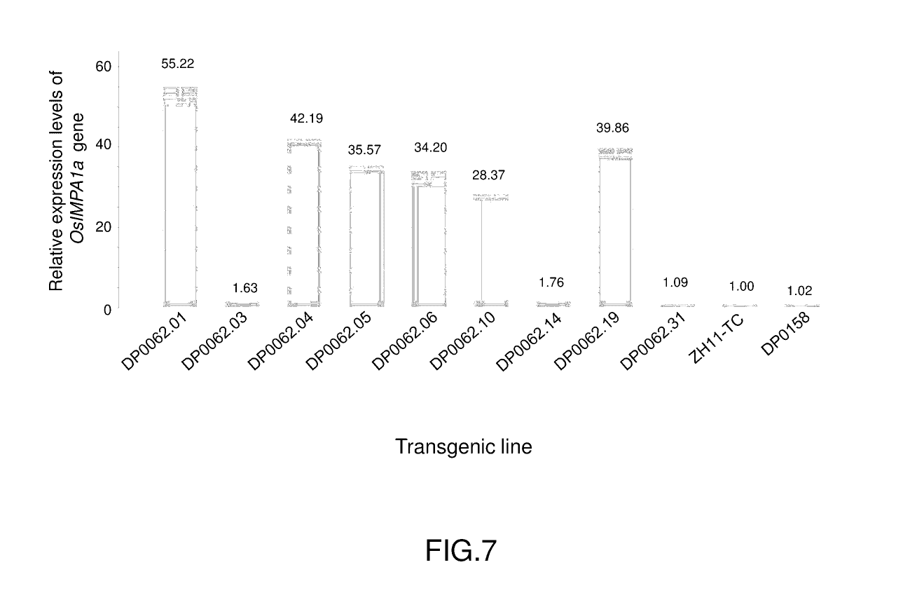

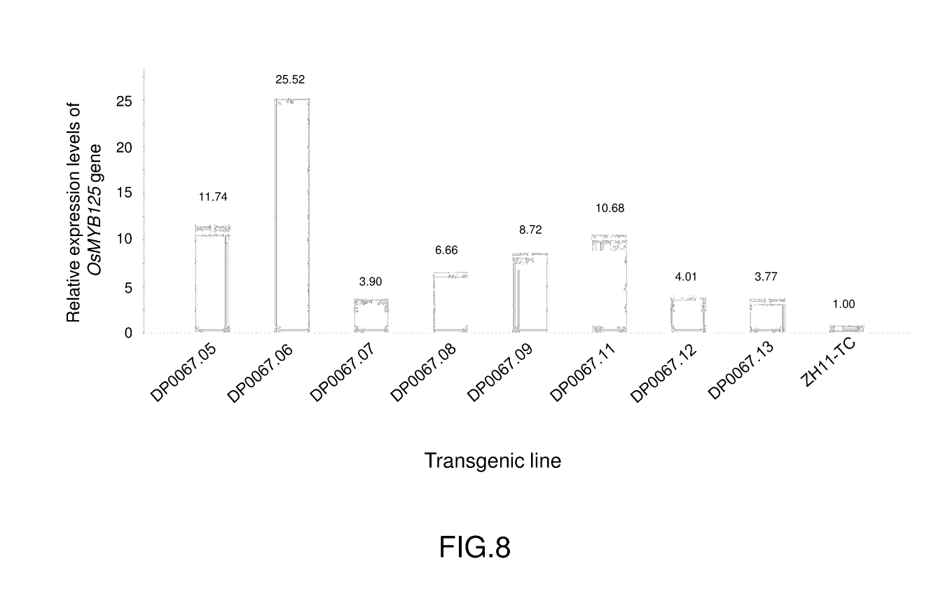

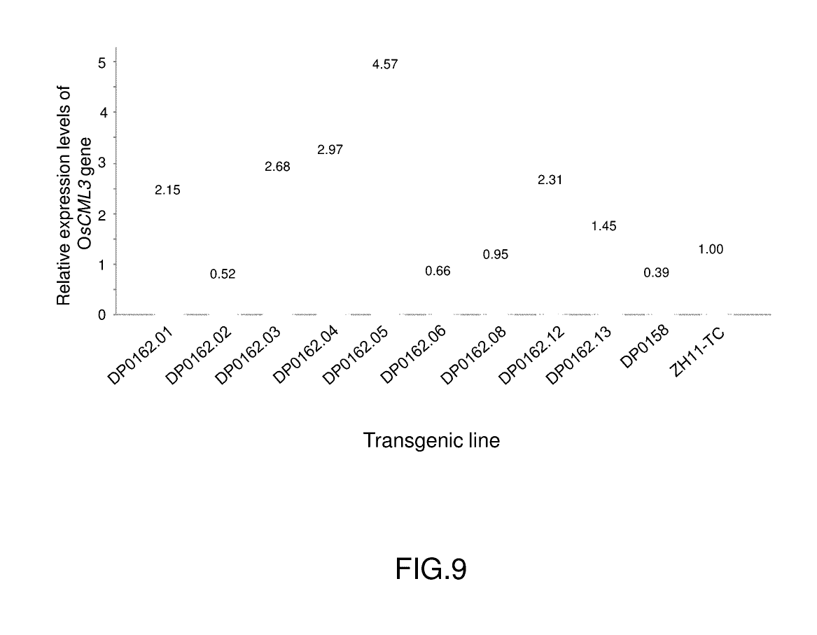

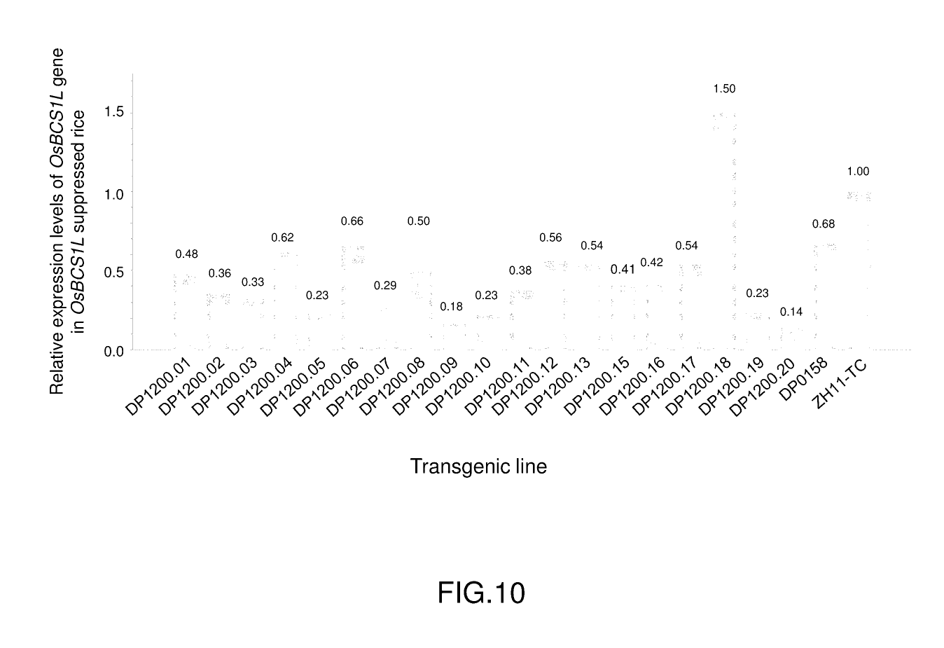

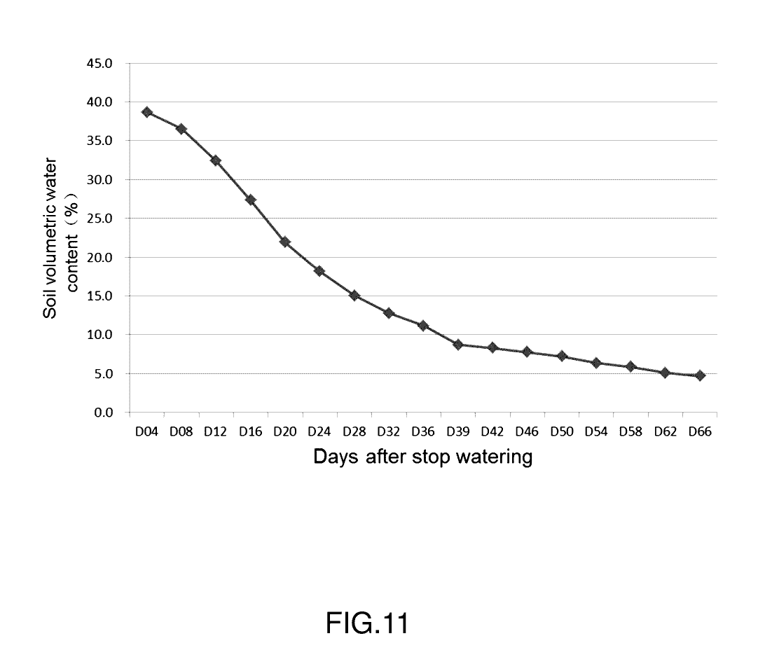

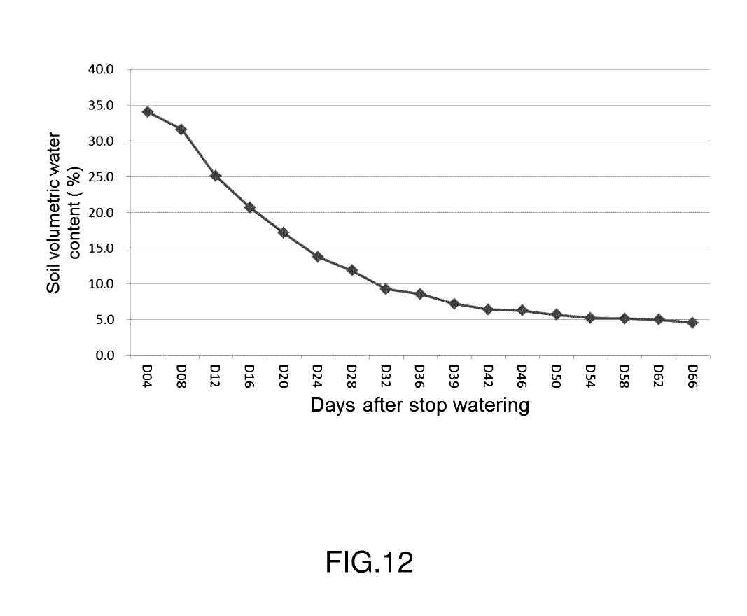

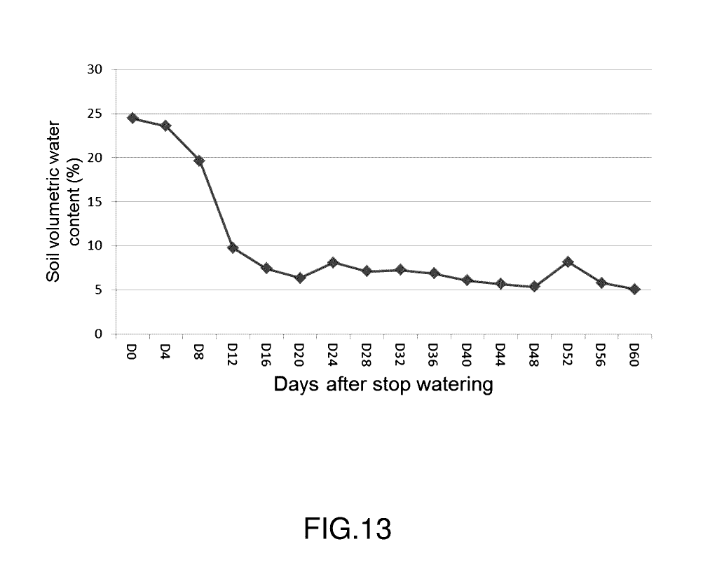

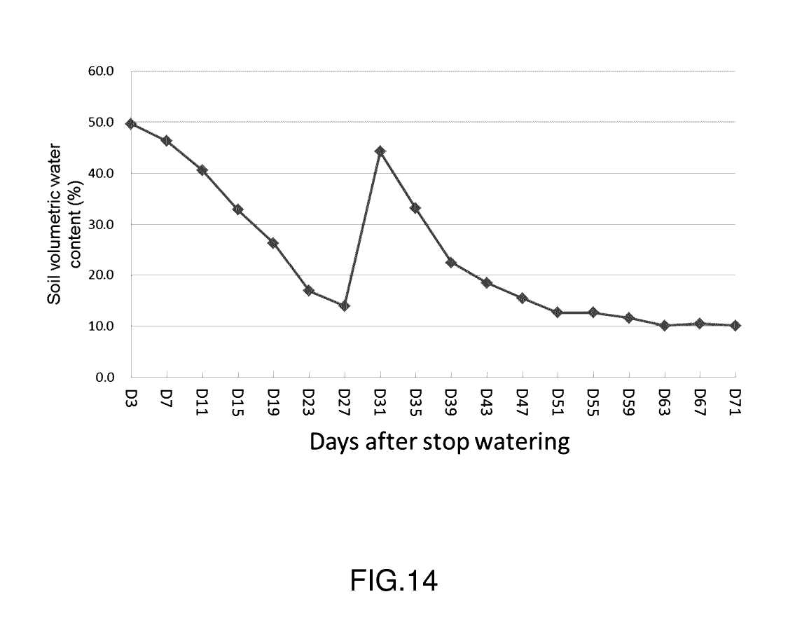

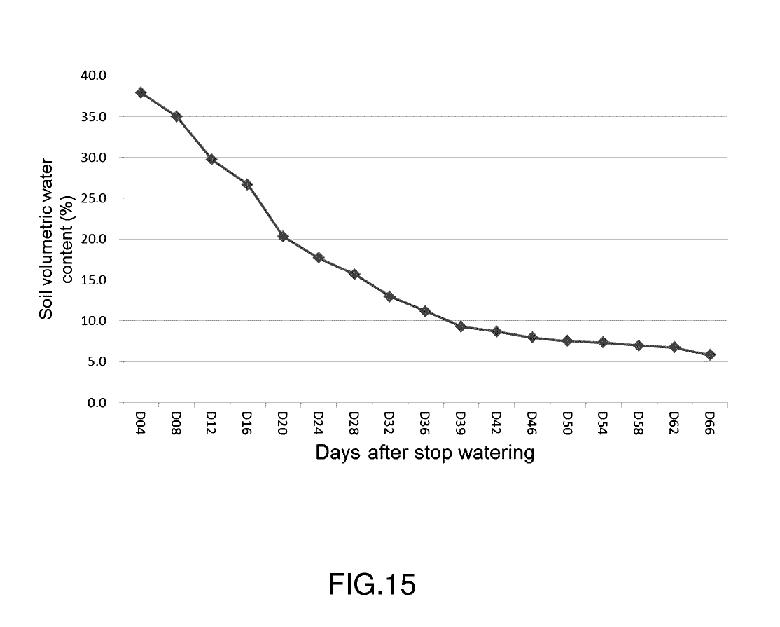

As shown in FIG. 3, the expression levels of OsBCS1L transgene in the leaves of transgenic rice plants were more than that in bulk null and ZH11-TC controls. The leaves were collected from rice plants which were treated by drought stress and were at heading stage.

### Example 4

### Drought Screening of Transgenic Rice Plants

The transgenic rice plants were screened in greenhouse drought assays. Two types of lamps were provided as light source, i.e. sodium lamp and metal halide lamp with the ratio of 1:1. Lamps provided the 16 h/8 h period of day/night, and were placed approximately 1.5 m above the seedbed. The light intensity 30 cm above the seedbed was measured as 10,000-20,000 lx in sunny day, while 6,000-10,000 lx in cloudy day, the relative humidity ranged from 30% to 90%, and the temperature ranged from 20 to 35° C.

Drought screening method:

T₂ Transgenic seedswere sterilized by 800 ppm carbendazol for 8 h at 32° C. and washed 3-5 times with distilled water, then soaked in water for 16 h at 32° C., germinated for 18 h at 35-37° C. in an incubator. The germinated seeds were sowed in one tray or pot filled with mixture of organic soil (FangJie soil from Beijing HuiYeShengDa Center), vermiculite (Beijing QingYuanShiJi Garden Center) and sand (Beijing Shuitun Construction Material Market) (V:V:V=3:3:2). The seedlings were grown under normal greenhouse condition and watered by modified IRRI solution. After all the seedlings grew to 3-leaf stage, watering was stopped and the trays were kept in a dry place until the leaves became dry and curved (approximately 9-15 days depending on the seasons). The trays were transferred into water pool to recover the seedlings for 5-7 days, and then plants were scored for the degree of recovery. The following scoring system was used: more than half green stem=1, more than two third green leaf=1, less than two third but more than one third green leaf=0.5, less than one third green leaf=0.2, no green leaf or less than half green stem=0. The recovery degree was the sum of the score of the green tissues, and the data were statistically analyzed using Mixed Model. The events which showed significant better than controls (p<0.05) were considered as positive ones. Survival rate (percentage of survived plants over the total plant number) was also used as a parameter for drought screening.

Three experiment designs were used. (1) The event null which is segregated from hemizygous plants used as control. Two transgenic rice plants and their event null plants were planted in pot (8×8×8 cm), and rice plants from each event were planted in 8 pot. (2) Latin Square design was used, and the total 16 plants for each event grew in different positions of the tray. The wild-type control (Zhonghua 11) from tissue culture procedure (ZH11-TC) and/or empty vector (DP0158) transgenic control in the same were used as controls. Several positive control (a drought tolerant variety, Mianhui 501) and negative control (a drought sensitive variety, Dongbeiyin 2) seedlings also were planted in the same tray. (3) Randomized block design was used for confirming the observation of the transformed rice from construct level. 9-12 transgenic events from the same construct were planted in one experimental unit to evaluate the transgene at construct level by Mixed Model considering construct, event and environment effects. If the survival rates or recovery degrees of the transgenic rice plants were significantly greater than control (p<0.05), the gene was considered having drought tolerant function.

GH drought assavresults:

### 1) DP0008 Transgenic Rice

Eleven OsDN-DTP2-transgenic events were tested by drought stress, and plated on different trays. ZH11-TC plants in the same tray were used as their corresponding controls. As shown in Table 6, 9 events showed higher survival rates and recovery degrees, and 6 events had significantly higher average recovery degrees than that of ZH11-TC, indicating that the OsDN-DTP2-transgenic rice plants had improved drought tolerance at seedling stage.

TABLE 6
Enhanced drought tolerance of OsDN-DTP2-transgenic rice
plants at T₂ generation under greenhouse conditions

| | Number | | Sur- | | | |
|---|---|---|---|---|---|---|
| | of sur- | Number | vival | Average | | |
| | vived | of total | rate | recovery | p- | p ≦ |
| Event ID | plants | plants | (%) | degree | value | 0.05 |
| DP0008.23 | 14 | 16 | 87.5 | 1.09 | 0.4125 | |
| ZH11-TC | 14 | 16 | 87.5 | 1.25 | | |
| DP0008.27 | 11 | 16 | 68.8 | 0.7 | 0.7561 | |
| ZH11-TC | 8 | 16 | 50.0 | 0.63 | | |
| DP0008.31 | 5 | 16 | 31.3 | 0.82 | 0.0259 | Y |
| ZH11-TC | 2 | 15 | 13.3 | 0.33 | | |
| DP0008.32 | 9 | 16 | 56.3 | 0.63 | 0.0598 | |
| ZH11-TC | 2 | 16 | 12.5 | 0.34 | | |
| DP0008.38 | 5 | 16 | 31.3 | 0.78 | 0.0162 | Y |
| ZH11-TC | 1 | 16 | 6.3 | 0.14 | | |
| DP0008.39 | 14 | 16 | 87.5 | 1.33 | 0.0011 | Y |
| ZH11-TC | 6 | 16 | 37.5 | 0.50 | | |
| DP0008.43 | 14 | 16 | 87.5 | 1.49 | 0.0133 | Y |
| ZH11-TC | 8 | 16 | 50.0 | 0.84 | | |
| DP0008.45 | 15 | 16 | 93.8 | 2.64 | 0.0063 | Y |
| ZH11-TC | 8 | 16 | 50.0 | 1.34 | | |
| DP0008.47 | 11 | 16 | 68.8 | 0.69 | 0.0005 | Y |
| ZH11-TC | 1 | 16 | 6.3 | 0.06 | | |
| DP0008.48 | 4 | 16 | 25.0 | 0.25 | 0.7278 | |
| ZH11-TC | 3 | 16 | 18.8 | 0.19 | | |

### 2) DP0014 Transgenic Rice

For OsMRP10-transgenic rice, 10 events and their event null rice plants were tested in the first experiment. The event null were used as their controls. Table 7 shows 6 events exhibited higher survival rates and recovery degrees than their corresponding controls, and other 3 events exhibited equal survival rates and higher recovery degrees. Two events exhibited significantly higher recovery degrees than their control. These results indicate that OsMRP10-transgenic rice plants had improved drought tolerance at seedling stage.

Construct level design was used in the second experiment.Nine events were tested. As shown in Table 8, 70 of 108 seedlings survived after drought stress, and the survival rate and recovery degree of OsMRP10-tansgenic rice was higher than DP0158 control and significantly higher than that of ZH11-TC control. These results further demonstrate that OsMRP10 gene plays a role in enhancing drought tolerance in plant.

TABLE 7
Enhanced drought tolerance of OsMRP10-transgenic rice plants
at T₂ generation under greenhouse conditions (1^{st} experiment)

| | Number | | Sur- | | | |
|---|---|---|---|---|---|---|
| | of sur- | Number | vival | Average | | |
| | vived | of total | rate | recovery | p- | p ≦ |
| Event ID | plants | plants | (%) | degree | value | 0.05 |
| DP0014.05 | 10 | 12 | 83.3 | 1.73 | 0.0741 | |
| DP0014.05-Null | 9 | 12 | 75.0 | 1.06 | | |
| DP0014.09 | 15 | 16 | 93.8 | 1.01 | 0.0144 | Y |
| DP0014.09-Null | 7 | 16 | 43.8 | 0.50 | | |
| DP0014.12 | 14 | 14 | 100.0 | 1.61 | 0.5822 | |
| DP0014.12-Null | 13 | 14 | 92.9 | 1.51 | | |
| DP0014.13 | 15 | 16 | 93.8 | 1.09 | 0.2368 | |
| DP0014.13-Null | 9 | 16 | 56.3 | 0.83 | | |
| DP0014.14 | 11 | 12 | 91.7 | 1.31 | 0.7910 | |
| DP0014.14-Null | 12 | 12 | 100.0 | 1.36 | | |
| DP0014.16 | 12 | 12 | 100.0 | 1.12 | 0.0579 | |
| DP0014.16-Null | 8 | 12 | 66.7 | 0.71 | | |
| DP0014.17 | 15 | 16 | 93.8 | 1.09 | 0.2525 | |
| DP0014.17-Null | 14 | 16 | 87.5 | 0.89 | | |
| DP0014.19 | 15 | 16 | 93.8 | 1.11 | 0.8898 | |
| DP0014.19-Null | 16 | 16 | 100.0 | 1.10 | | |
| DP0014.20 | 12 | 12 | 100.0 | 1.91 | 0.0205 | Y |
| DP0014.20-Null | 12 | 12 | 100.0 | 1.13 | | |
| DP0014.21 | 12 | 12 | 100.0 | 1.41 | 0.9319 | |
| DP0014.21-Null | 12 | 12 | 100.0 | 1.39 | | |

TABLE 8
Enhanced drought tolerance of OsMRP10-transgenic
rice plants at T₂ generation under greenhouse conditionsat
construct level (2^{nd} experiment)

| | Number | | Sur- | | | |
|---|---|---|---|---|---|---|
| | of sur- | Number | vival | Average | | |
| | vived | of total | rate | recovery | p- | p ≦ |
| Construct ID | plants | plants | (%) | degree | value | 0.05 |
| DP0014 | 70 | 108 | 64.8 | 0.70 | 0.4390 | |
| DP0158 | 7 | 12 | 58.3 | 0.58 | | |
| DP0014 | 70 | 108 | 64.8 | 0.70 | 0.0392 | Y |
| ZH11-TC | 10 | 24 | 41.7 | 0.47 | | |

### 3) DP0055 Transgenic Rice

In the first experiment, Latin square design was used, and 12 OsGSTU35-transgenic events were tested. The different events were planted in different trays, and the ZH11-TC and DP0158 seedlings in the same tray were used as their corresponding controls. Table 9 shows that 10 events had higher survival rate and significantly higher recovery degrees than ZH11-TC control. When compared with DP0158 control, 10 events exhibited higher survival rates and average recovery degrees, and 5 events had significantly higher recovery degrees. These results indicate that OsGSTU35-transgenic rice had enhanced drought tolerance.

Construct level design was used in the second experiment. Nine events were tested. As shown in Table 10, 52 of 108 seedlings survived after drought stress, and the survival rate and recovery degree of OsGSTU35-tansgenic rice was higher than DP0158 control and significantly higher than that of ZH11-TC control. These results further demonstrate that OsGSTU35gene plays a role in enhancing drought tolerance in plant.

TABLE 9
Enhanced drought tolerance of OsGSTU35-transgenic rice plants
at T₂ generation under greenhouse conditions (1^{st} experiment)

| | Number | | Sur- | | | |
|---|---|---|---|---|---|---|
| | of sur- | Number | vival | Average | | |
| | vived | of total | rate | recovery | p- | p ≦ |
| Event ID | plants | plants | (%) | degree | value | 0.05 |
| DP0055.01 | 6 | 16 | 37.5 | 0.45 | 0.0379 | Y |
| ZH11-TC | 1 | 16 | 6.3 | 0.06 | | |
| DP0055.03 | 8 | 16 | 50.0 | 0.50 | 0.0079 | Y |
| ZH11-TC | 2 | 16 | 12.5 | 0.13 | | |
| DP0055.05 | 11 | 16 | 68.8 | 0.76 | 0.0011 | Y |
| ZH11-TC | 2 | 16 | 12.5 | 0.13 | | |
| DP0055.07 | 15 | 16 | 93.8 | 1.41 | 0.0003 | Y |
| ZH11-TC | 5 | 16 | 31.3 | 0.38 | | |
| DP0055.09 | 15 | 16 | 93.8 | 2.86 | 0.0140 | Y |
| ZH11-TC | 9 | 15 | 60.0 | 1.56 | | |
| DP0055.17 | 15 | 16 | 93.8 | 1.60 | 0.0000 | Y |
| ZH11-TC | 5 | 16 | 31.3 | 0.41 | | |
| DP0055.18 | 10 | 16 | 62.5 | 1.37 | 0.0031 | Y |
| ZH11-TC | 0 | 16 | 0.0 | 0.00 | | |
| DP0055.19 | 16 | 16 | 100.0 | 2.93 | 0.0018 | Y |
| ZH11-TC | 6 | 16 | 37.5 | 1.26 | | |
| DP0055.20 | 12 | 16 | 75.0 | 1.04 | 0.0195 | Y |
| ZH11-TC | 3 | 16 | 18.8 | 0.28 | | |
| DP0055.22 | 14 | 16 | 87.5 | 2.83 | 0.0002 | Y |
| ZH11-TC | 5 | 16 | 31.3 | 0.96 | | |

TABLE 10
Enhanced drought tolerance of OsGSTU35-transgenic
rice plants at T₂ generation under greenhouse conditionsat
construct level (2^{nd} experiment)

| | Number | | Sur- | | | |
|---|---|---|---|---|---|---|
| | of sur- | Number | vival | Average | | |
| | vived | of total | rate | recovery | p- | p ≦ |
| Construct ID | plants | plants | (%) | degree | value | 0.05 |
| DP0055 | 52 | 108 | 48.1 | 0.49 | 0.0534 | |
| ZH11-TC | 6 | 24 | 25.0 | 0.26 | | |
| DP0055 | 52 | 108 | 48.1 | 0.49 | 0.3376 | |
| DP0158 | 4 | 12 | 33.3 | 0.33 | | |

### 4) DP0060 Transgenic Rice

Latin square design was used, 12 OsCML1-transgenic events were tested. The different events were planted in different trays, and the ZH11-TC and DP0158 seedlings in the same tray were used as their corresponding controls. Table 11 shows that 10 events had higher survival rate and higher recovery degrees than ZH11-TC control, and 9 events had significantly higher recovery degrees. When compared with DP0158 control,9 events exhibited higher survival rates and average recovery degrees, and 6 events had significantly higher recovery degrees. These results indicate that OsCML1-transgenic rice had enhanced drought tolerance.

TABLE 11
Enhanced drought tolerance of OsCML1-transgenic rice
plants at T₂ generaton under greenhouse conditions

| | Number | | Sur- | | | |
|---|---|---|---|---|---|---|
| | of sur- | Number | vival | Average | | |
| | vived | of total | rate | recovery | p- | p ≦ |
| Event ID | plants | plants | (%) | degree | value | 0.05 |
| DP0060.03 | 7 | 15 | 46.7 | 0.50 | 0.0079 | Y |
| ZH11-TC | 2 | 16 | 12.5 | 0.13 | | |
| DP0060.04 | 12 | 16 | 75.0 | 0.95 | 0.0000 | Y |
| ZH11-TC | 2 | 16 | 12.5 | 0.13 | | |
| DP0060.06 | 13 | 16 | 81.3 | 1.25 | 0.0141 | Y |
| ZH11-TC | 9 | 16 | 56.3 | 0.66 | | |
| DP0060.07 | 11 | 16 | 68.8 | 1.16 | 0.0046 | Y |
| ZH11-TC | 5 | 16 | 31.3 | 0.38 | | |
| DP0060.09 | 10 | 16 | 62.5 | 2.00 | 0.3930 | |
| ZH11-TC | 9 | 15 | 60.0 | 1.56 | | |
| DP0060.10 | 14 | 16 | 87.5 | 1.44 | 0.0000 | Y |
| ZH11-TC | 5 | 16 | 31.3 | 0.41 | | |
| DP0060.11 | 10 | 16 | 62.5 | 1.49 | 0.0014 | Y |
| ZH11-TC | 0 | 16 | 0.0 | 0.00 | | |
| DP0060.13 | 14 | 16 | 87.5 | 2.66 | 0.0079 | Y |
| ZH11-TC | 6 | 16 | 37.5 | 1.26 | | |
| DP0060.14 | 15 | 16 | 93.8 | 1.62 | 0.0000 | Y |
| ZH11-TC | 3 | 16 | 18.8 | 0.28 | | |
| DP0060.17 | 13 | 16 | 81.3 | 2.74 | 0.0003 | Y |
| ZH11-TC | 5 | 16 | 31.3 | 0.96 | | |

### 5) DP0062 Transgenic Rice

Latin square design was used, 12 OsIMPA1a-transgenic events were tested. The different events were planted in different tray, and the ZH11-TC and DP0158 seedlings in the same tray were used as their corresponding controls. Table 12 shows that 10 events had higher survival rate and higher recovery degrees than ZH11-TC control, and 5 events hadsignificantly higher recovery degrees. When compared with DP0158 control, 9 events exhibited higher survival rates and 7 events had higher average recovery degrees, and 3 events had significantly higher recovery degrees. These results indicate that OsIMPA1a-transgenic rice had enhanced drought tolerance.

Construct level design was used in the second experiment. Nine events were tested. As shown in Table 13, the survival rate and recovery degree of OsIMFA1a-tansgenic rice was higher than DP0158 control and of ZH11-TC control. These results further demonstrate that OsIMFA1a gene plays a role in enhancing drought tolerance in plant.

TABLE 12
Enhanced drought tolerance of OsIMPA1a-transgenic rice plants
at T₂ generation under greenhouse conditions (1^{st} experiment)

| | Number | | Sur- | | | |
|---|---|---|---|---|---|---|
| | of sur- | Number | vival | Average | | |
| | vived | of total | rate | recovery | p- | p ≦ |
| Event ID | plants | plants | (%) | degree | value | 0.05 |
| DP0062.01 | 15 | 16 | 93.8 | 1.33 | 0.0000 | Y |
| ZH11-TC | 1 | 16 | 6.3 | 0.06 | | |
| DP0062.03 | 2 | 16 | 12.5 | 0.13 | 1.0000 | |
| ZH11-TC | 2 | 16 | 12.5 | 0.13 | | |
| DP0062.04 | 10 | 16 | 62.5 | 0.69 | 0.0034 | Y |
| ZH11-TC | 2 | 16 | 12.5 | 0.13 | | |
| DP0062.05 | 11 | 16 | 68.8 | 0.81 | 0.5340 | |
| ZH11-TC | 9 | 16 | 56.3 | 0.66 | | |
| DP0062.06 | 13 | 16 | 81.3 | 1.03 | 0.0173 | Y |
| ZH11-TC | 5 | 16 | 31.3 | 0.38 | | |
| DP0062.10 | 6 | 15 | 40.0 | 0.66 | 0.9271 | |
| ZH11-TC | 8 | 16 | 50.0 | 0.64 | | |
| DP0062.14 | 14 | 16 | 87.5 | 2.11 | 0.2840 | |
| ZH11-TC | 9 | 15 | 60.0 | 1.56 | | |
| DP0062.19 | 14 | 16 | 87.5 | 1.23 | 0.0006 | Y |
| ZH11-TC | 5 | 16 | 31.3 | 0.41 | | |
| DP0062.23 | 5 | 16 | 31.3 | 0.43 | 0.3336 | |
| ZH11-TC | 0 | 16 | 0.0 | 0.00 | | |
| DP0062.25 | 13 | 16 | 81.3 | 2.14 | 0.0882 | |
| ZH11-TC | 6 | 16 | 37.5 | 1.26 | | |
| DP0062.27 | 11 | 16 | 68.8 | 0.81 | 0.0957 | |
| ZH11-TC | 3 | 16 | 18.8 | 0.28 | | |
| DP0062.31 | 15 | 15 | 100.0 | 3.70 | 0.0000 | Y |
| ZH11-TC | 5 | 16 | 31.3 | 0.96 | | |

TABLE 13
Enhanced drought tolerance of OsIMPA1a-transgenic
rice plants at T₂ generation under greenhouse conditionsat
construct level (2^{nd} experiment)

| | Number | | Sur- | | | |
|---|---|---|---|---|---|---|
| | of sur- | Number | vival | Average | | |
| | vived | of total | rate | recovery | p- | p ≦ |
| Construct ID | plants | plants | (%) | degree | value | 0.05 |
| DP0062 | 66 | 108 | 61.1 | 0.64 | 0.4952 | |
| ZH11-TC | 12 | 24 | 50.0 | 0.55 | | |
| DP0062 | 66 | 108 | 61.1 | 0.64 | 0.0864 | |
| DP0158 | 4 | 12 | 33.3 | 0.33 | | |

### 6) DP0067 Transgenic Rice

For OsMYB125-transgenic rice, 9 events and their event null segregated from the hemizygous rice plants were tested and 2 seedlings of each event were planted in one pot (8×8×8 cm) in the first experiment. The event null were used as their controls. Table 14shows 8 events exhibited higher survival rates and recovery degrees than their corresponding controls, and 3 events exhibited significantly higher recovery degrees than their control. These results indicate that OsMYB125-transgenic rice plants had improved drought tolerance at seedling stage.

Latin square design was used in the second experiment, 11 OsMYB125-transgenic events were tested. The different events were planted in different tray, and the ZH11-TC and DP0158 seedlings in the same tray were used as their corresponding controls. Table 15 shows that 8events had higher survival rate and higher recovery degrees than ZH11-TC control, and 5 events hadsignificantly higher recovery degrees. When compared with DP0158 control, 9 events exhibited higher survival rates and higher average recovery degrees, and 5 events had significantly higher recovery degrees. These results further indicate that OsMYB125 -transgenic rice had enhanced drought tolerance.

TABLE 14
Enhanced drought tolerance of OsMYB125-transgenic rice plants
at T₂ generation under greenhouse conditions (1^{st} experiment)

| | Number | | Sur- | | | |
|---|---|---|---|---|---|---|
| | of sur- | Number | vival | Average | | |
| | vived | of total | rate | recovery | p- | p ≦ |
| Event ID | plants | plants | (%) | degree | value | 0.05 |
| DP0067.03 | 3 | 6 | 50.0 | 0.83 | 0.7418 | |
| DP0067.03-Null | 3 | 6 | 50.0 | 0.67 | | |
| DP0067.05 | 7 | 8 | 87.5 | 0.90 | 0.0098 | Y |
| DP0067.05-Null | 1 | 8 | 12.5 | 0.13 | | |
| DP0067.06 | 9 | 10 | 90.0 | 1.26 | 0.0016 | Y |
| DP0067.06-Null | 3 | 10 | 30.0 | 0.40 | | |
| DP0067.07 | 6 | 6 | 100.0 | 1.53 | 0.2075 | |
| DP0067.07-Null | 3 | 6 | 50.0 | 0.70 | | |
| DP0067.10 | 8 | 12 | 66.7 | 0.85 | 0.0505 | |
| DP0067.10-Null | 5 | 12 | 41.7 | 0.42 | | |
| DP0067.11 | 10 | 12 | 83.3 | 1.58 | 0.2522 | |
| DP0067.11-Null | 9 | 12 | 75.0 | 1.29 | | |
| DP0067.12 | 9 | 10 | 90.0 | 1.50 | 0.0533 | |
| DP0067.12-Null | 6 | 10 | 60.0 | 1.05 | | |
| DP0067.13 | 10 | 14 | 71.4 | 0.86 | 0.0152 | Y |
| DP0067.13-Null | 4 | 14 | 28.6 | 0.29 | | |

TABLE 15
Enhanced drought tolerance of OsMYB125-transgenic rice plants
at T₂ generation under greenhouse conditions (2^{nd} experiment)

| | Number | | Sur- | | | |
|---|---|---|---|---|---|---|
| | of sur- | Number | vival | Average | | |
| | vived | of total | rate | recovery | p- | p ≦ |
| Event ID | plants | plants | (%) | degree | value | 0.05 |
| DP0067.01 | 7 | 16 | 43.8 | 0.71 | 0.4521 | |
| ZH11-TC | 4 | 16 | 25.0 | 0.50 | | |
| DP0067.02 | 10 | 16 | 62.5 | 0.81 | 0.4436 | |
| ZH11-TC | 9 | 16 | 56.3 | 0.60 | | |
| DP0067.05 | 14 | 16 | 87.5 | 0.91 | 0.0085 | Y |
| ZH11-TC | 8 | 16 | 50.0 | 0.50 | | |
| DP0067.07 | 12 | 16 | 75.0 | 2.78 | 0.0091 | Y |
| ZH11-TC | 5 | 16 | 31.3 | 1.17 | | |
| DP0067.08 | 4 | 16 | 25.0 | 0.25 | 0.4393 | |
| ZH11-TC | 2 | 16 | 12.5 | 0.13 | | |
| DP0067.09 | 11 | 16 | 68.8 | 0.69 | 0.0035 | Y |
| ZH11-TC | 3 | 16 | 18.8 | 0.19 | | |
| DP0067.12 | 15 | 16 | 93.8 | 1.06 | 0.0000 | Y |
| ZH11-TC | 0 | 16 | 0.0 | 0.00 | | |
| DP0067.13 | 8 | 16 | 50.0 | 0.73 | 0.0000 | Y |
| ZH11-TC | 0 | 16 | 0.00 | 0.00 | | |

### 7) DP0162 Transgenic Rice

Latin square design was used in the first experiment, 12 OsCML3-transgenic events were tested. The different events were planted in different tray, and the ZH11-TC and DP0158 seedlings in the same tray were used as their corresponding controls. Table 16 shows that 9 events had higher survival rates and higher recovery degrees than ZH11-TC control, and 7 events hadsignificantly higher recovery degrees. When compared with DP0158 control, 9 events exhibited higher survival rates and higher average recovery degrees, and 5 events had significantly higher recovery degrees. These results indicate that OsCML3 -transgenic rice had enhanced drought tolerance.

Construct level design was used in the second experiment.Nine events were tested. As shown in Table 17, all of the tested OsCML3-tansgenic rice exhibited higher survival rate and significantly higher recovery degree than DP0158 and of ZH11-TC controls. These results further demonstrate that OsCML3 gene plays a role in enhancing drought tolerance in plant.

TABLE 16
Enhanced drought tolerance of OsCML3-transgenic rice plants at T₂ generation
under greenhouse conditions (1^{st} experiment)

| | Number of | | | Average | | |
|---|---|---|---|---|---|---|
| | survived | Number of | Survival rate | recovery | | |
| Event ID | plants | total plants | (%) | degree | p-value | p ≦ 0.05 |
| DP0162.01 | 16 | 16 | 100.0 | 1.42 | 0.0111 | Y |
| ZH11-TC | 10 | 16 | 62.5 | 0.89 | | |
| DP0162.02 | 13 | 16 | 81.3 | 1.25 | 0.0003 | Y |
| ZH11-TC | 2 | 16 | 12.5 | 0.13 | | |
| DP0162.03 | 14 | 16 | 87.5 | 2.03 | 0.0000 | Y |
| ZH11-TC | 2 | 16 | 12.5 | 0.25 | | |
| DP0162.04 | 13 | 16 | 81.3 | 1.71 | 0.0000 | Y |
| ZH11-TC | 2 | 16 | 12.5 | 0.19 | | |
| DP0162.05 | 12 | 16 | 75.0 | 1.47 | 0.0000 | Y |
| ZH11-TC | 1 | 15 | 6.7 | 0.13 | | |
| DP0162.06 | 12 | 16 | 75.0 | 1.29 | 0.0269 | Y |
| ZH11-TC | 4 | 15 | 26.7 | 0.56 | | |
| DP0162.08 | 6 | 16 | 37.5 | 0.43 | 0.8371 | |
| ZH11-TC | 6 | 15 | 40.0 | 0.38 | | |
| DP0162.09 | 8 | 16 | 50.0 | 0.97 | 0.4278 | |
| ZH11-TC | 12 | 16 | 75.0 | 1.22 | | |
| DP0162.10 | 16 | 16 | 100.0 | 1.04 | 0.0000 | Y |
| ZH11-TC | 4 | 16 | 25.0 | 0.25 | | |
| DP0162.12 | 9 | 16 | 56.3 | 1.00 | 0.2107 | |
| ZH11-TC | 6 | 16 | 37.5 | 0.67 | | |
| DP0162.13 | 8 | 16 | 50.0 | 0.66 | 0.7525 | |
| ZH11-TC | 8 | 16 | 50.0 | 0.61 | | |
| DP0162.14 | 11 | 16 | 68.8 | 0.74 | 0.2636 | |
| ZH11-TC | 9 | 16 | 56.3 | 0.56 | | |

TABLE 17
Enhanced drought tolerance of OsCML3-transgenic rice plants at T₂ generation
under greenhouse conditions at construct level (2^{nd} experiment)

| | Number of | | | Average | | |
|---|---|---|---|---|---|---|
| | survived | Number of | Survival rate | recovery | | |
| Construct ID | plants | total plants | (%) | degree | p-value | p ≦ 0.05 |
| DP0162 | 65 | 108 | 60.2 | 0.85 | 0.0158 | Y |
| ZH11-TC | 9 | 24 | 37.5 | 0.43 | | |
| DP0162 | 65 | 108 | 60.2 | 0.85 | 0.0471 | Y |
| DP0158 | 4 | 12 | 33.3 | 0.42 | | |

### 8) DP0196 OsBCS1L-Transgenic Rice

For OsBCS1L-transgenic rice, 11 events and their event null segregated from the hemizygous rice plants were tested and 2 seedlings of each event were planted in one pot (8×8×8 cm) in the first experiment. The event null were used as their controls. Table 18 shows 6 events exhibited lower survival rates and recovery degrees than their corresponding controls, and 3 events exhibited significantly lower recovery degrees than their control. These results indicate that OsBCS1L-transgenic rice plants showed drought sensitive at seedling stage.

Construct level design was used in the second experiment. Nine events were tested. As shown in Table 19, all of the tested OsBCS1L-tansgenic rice exhibited lower survival rate and significantly lower recovery degree than DP0158 and of ZH11-TC controls. Further analysis at transgenic level indicated that all 9 events showed lower survival rates and recovery degrees than either ZH11-TC or DP0158 control, and 6 events showed significantly lower recovery degrees than that of ZH11-TC control and 9 events showed significantly lower recovery degrees than that of DP0158 controls. These results further and clearly demonstrate that OsBCS1L gene plays a role in reducing drought tolerance activity in plant.

TABLE 18
Drought tolerance assay of OsBCS1L-transgenic rice plants at T₂ generation
under greenhouse conditions (1^{st} experiment)

| | Number of | | | Average | | |
|---|---|---|---|---|---|---|
| | survived | Number of | Survival rate | recovery | | |
| Event ID | plants | total plants | (%) | degree | p-value | p ≦ 0.05 |
| DP0196.02 | 5 | 10 | 50.00 | 0.99 | 0.3373 | |
| DP0196.02-Null | 8 | 10 | 80.00 | 1.62 | | |
| DP0196.04 | 10 | 12 | 83.33 | 1.20 | 0.0079 | Y |
| DP0196.04-Null | 11 | 12 | 91.67 | 1.91 | | |
| DP0196.05 | 5 | 14 | 35.71 | 0.43 | 0.0002 | Y |
| DP0196.05-Null | 10 | 14 | 71.43 | 1.26 | | |
| DP0196.06 | 10 | 12 | 83.33 | 1.45 | 0.9302 | |
| DP0196.06-Null | 9 | 12 | 75.00 | 1.43 | | |
| DP0196.12 | 8 | 14 | 57.14 | 1.17 | 0.4498 | |
| DP0196.12-Null | 8 | 14 | 57.14 | 0.90 | | |
| DP0196.13 | 4 | 10 | 40.00 | 0.85 | 0.1369 | |
| DP0196.13-Null | 8 | 10 | 80.00 | 1.30 | | |
| DP0196.17 | 4 | 12 | 33.33 | 0.62 | 0.0210 | Y |
| DP0196.17-Null | 11 | 12 | 91.67 | 1.79 | | |
| DP0196.22 | 4 | 6 | 66.67 | 1.12 | 0.1862 | |
| DP0196.22-Null | 5 | 6 | 83.33 | 1.73 | | |

TABLE 19
Drought tolerance assay of OsBCS1L-transgenic rice plants at T₂
generation under greenhouse conditions at construct level (2^{nd} experiment)

| | Number | | | | | |
|---|---|---|---|---|---|---|
| | of | Number of | Survival | Average | | |
| | survived | total | rate | recovery | | p ≦ |
| Event ID | plants | plants | (%) | degree | p-value | 0.05 |
| DP0196 | 53 | 108 | 49.1 | 0.54 | 0.0106 | Y |
| ZH11-TC | 19 | 24 | 79.2 | 0.90 | | |
| DP0196 | 53 | 108 | 49.1 | 0.54 | 0.0005 | Y |
| DP0158 | 11 | 12 | 91.7 | 1.18 | | |

In summary, the OsDN-DTP2, OsMRP10, OsGSTU35, OsCML1, OsIMPA1a, OsMYB125 and OsCML3-transgenic rice plants showed better survival rates and significantly greater recovery degrees compared to ZH11-TC, and/or DP0158 control plants. These results demonstrate that over-expression of OsDN-DTP2, OsMRP10, OsGSTU35, OsCML1, OsIMPA1a, OsMYB125 and OsCML3under constitutive promoter CaMV 35S increased the drought tolerance of rice plants. The OsBCS1L-transgenic rice plants exhibited drought sensitive phenotype.

### Example 5

### Field Drought Assays of Mature Transgenic Rice Plants

Flowering stage drought stress is an important problem in agriculture practice. The transgenic rice plants were further tested under field drought conditions. For the Field drought assays of mature rice plants, 9-12 transgenic events of each gene construct weretested. The T₂ seeds were first sterilized as described in Example 4. The germinated seeds were planted in a seedbed field. At 3-leaf stage, the seedlings were transplanted into the testing field, with 4 replicates and 10 plants per replicate for each transgenic event, and the 4 replicates were planted in the same block. ZH11-TC, DP0158 and Bulk Nullwere nearby the transgenic events in the same block, and were used as controls in the statistical analysis.

The rice plants were managed by normal practice using pesticides and fertilizers. Watering was stopped at the tillering stage, so as to give drought stress at flowering stage depending on the weather conditions (temperature and humidity). The soil water content was measured every 4 days at about 10 sites per block using TDR30 (Spectrum Technologies, Inc.).

Plant phenotypes were observed and recorded during the experiments. The phenotypes include heading date, leaf rolling degree, drought sensitivity (for OsBCS1L) and drought tolerance. Special attention was paid to leaf rolling degree at noontime. At the end of the growingseason, 6 representative plants of each transgenic event were harvested from the middle of the row per line, and grain weight per plant was measured. The grain weight data were statistically analyzed using mixed linear model. Positive transgenic events were selected based on the analysis (P<0.1).

### Field Drought Assay Results:

### 1) DP0008 Transgenic Rice

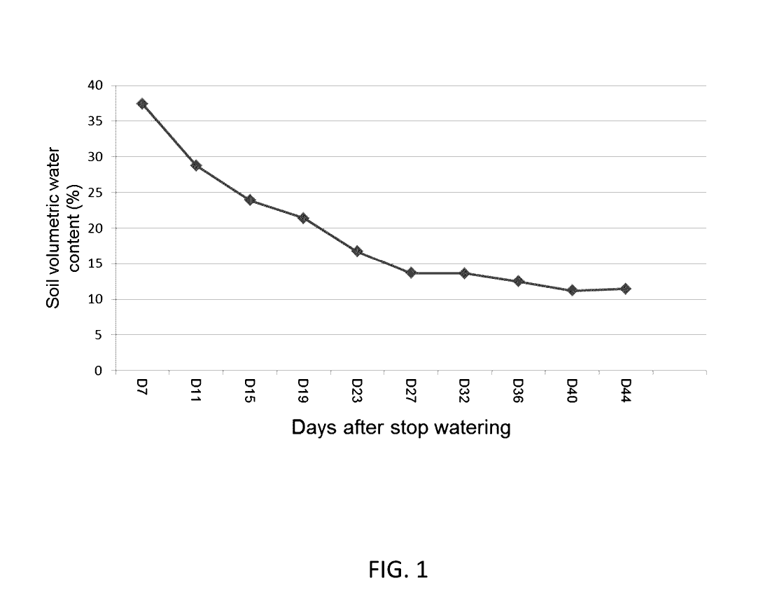

Fourteen OsDN-DTP2-transgenic events were tested in Hainan Province inthe first experiment, the event null and ZH11-TC rice plants planted nearby were used as control. Watering was stopped from panicle initiationstage Ilto seed maturity to produce heavier drought stress. The soil volumetric moisture content decreased from 38% to 10% during heading and maturation stage (FIG. 1). At the end of the planting season, 6 representative plants of each transgenic event were harvested from the middle of the row per line, and grain weight per plant was measured. As shown in Table 20, 5 events exhibited significantly higher grain yield per plant than that of their corresponding event null and higher than that of ZH11-TC controls. These results demonstrate that OsDN-DTP2-rice plant had greater grain yield per plant than control after drought stress.

TABLE 20
Grain yield assay of OsDN-DTP2-rice plants at T₂ generation under field drought
conditions

| | Number of | Number of | Grain yield | | | | |
|---|---|---|---|---|---|---|---|
| | survived | harvest | per plant | CK = Event Null | | CK = DP0005 | |
| Event ID | plants | plants | (g) | p-value | p ≦ 0.05 | p-value | p ≦ 0.05 |
| DP0008.14 | 40 | 24 | 10.21 | 0.019 | Y | 0.329 | |
| CK1 (DP0008.16) | 40 | 24 | 7.08 | | | | |
| DP0008.17.16 | 40 | 24 | 12.01 | 0.000 | Y | 0.004 | Y |
| CK1 (DP0008.22) | 40 | 24 | 5.61 | | | | |
| DP0008.18.32 | 40 | 24 | 11.70 | 0.001 | Y | 0.010 | Y |
| CK1 (DP0008.19) | 40 | 24 | 7.68 | | | | |
| DP0008.19.16 | 40 | 24 | 10.92 | 0.000 | Y | 0.083 | Y |
| CK1 (DP0008.19) | 40 | 24 | 6.33 | | | | |
| DP0008.20.26 | 40 | 24 | 11.85 | 0.000 | Y | 0.006 | Y |
| CK1 (DP0008.22) | 40 | 24 | 6.91 | | | | |
| CK2 (DP0005) | 40 | 24 | 9.30 | | | | |

### 2) DP0196 Transgenic Rice

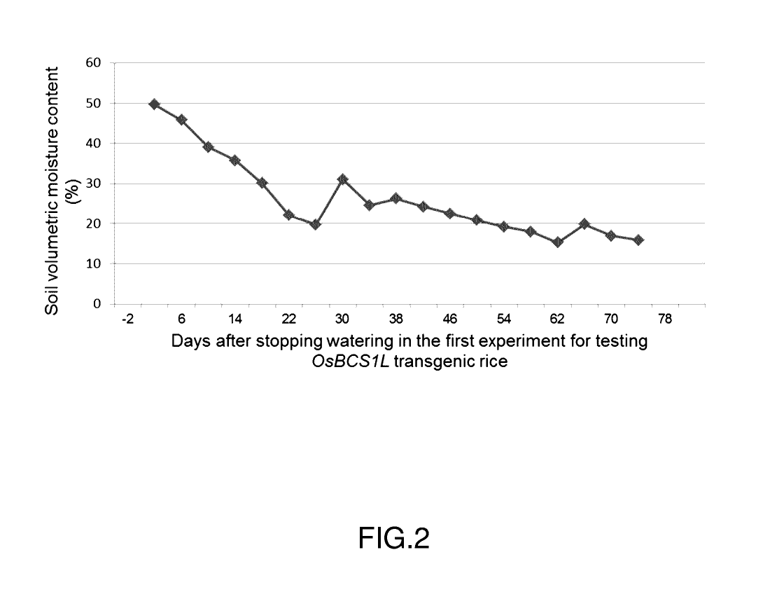

Eight OsBCSlL-transgenic events were tested in Beijing in the first experiment,and the bulknull (seeds segregated from hemizygous OsBCS1L-transgenic plants) and ZH11-TC rice plants planted nearby were used as control. Eight plants of each event were planted and repeated for 3 times. Watering was stopped from panicle initiation stage II to seed maturity to produce heavier drought stress. The soil volumetric moisture content decreased from 50% to 15% during heading and maturation stage (FIG. 2). At the end of the growingseason, about 5 representative plants of each transgenic event were harvested from the middle of the row per line, and grain weight per plant was measured. As shown in Table 21, 7 events exhibited lower grain yield per plant than that of the bulk null and ZH11-TC controls, and 3 events had significantly lower grain yield per plant. The 3 events (DP0196.04, DP0196.13 and DP0196.17) showed significant phenotype of leaf rolling and leaf drying during drought stress.These results demonstrate that OsBCS1L-rice plantis sensitive to drought, and over-expression of OsBCS1L reduced the grain yield per plant after drought stress at flowering stage.

TABLE 21
Grain yield assay of OsBCS1L-rice plants at T₂ generation under field drought
conditions

| | Number of | Number of | Grain yield | | | | |
|---|---|---|---|---|---|---|---|
| | survived | harvested | per plant | CK = ZH11-TC | | CK = Bulk Null | |
| Event ID | plants | plants | (g) | p-value | p ≦ 0.05 | p-value | p ≦ 0.05 |
| DP0196.04 | 24 | 10 | 0.58 | 0.000 | Y | 0.000 | Y |
| DP0196.05 | 24 | 16 | 3.76 | 0.832 | | 0.524 | |
| DP0196.06 | 24 | 16 | 2.55 | 0.080 | | 0.065 | |
| DP0196.07 | 24 | 16 | 5.22 | 0.081 | | 0.328 | |
| DP0196.09 | 24 | 16 | 3.05 | 0.249 | | 0.142 | |
| DP0196.12 | 16 | 10 | 2.79 | 0.199 | | 0.132 | |
| DP0196.13 | 24 | 10 | 1.65 | 0.002 | Y | 0.003 | Y |
| DP0196.17 | 24 | 6 | 1.93 | 0.009 | Y | 0.006 | Y |
| CK1(BN) | 24 | 15 | 4.39 | 0.590 | | 1.000 | |
| CK2(ZH11-TC) | 24 | 16 | 3.94 | | | | |

### Example 6

### Cold Assays of Transgenic Rice Plants UnderLow Temperature Conditions

Nine to twelve events per construct were tested for cold assay. T₂ Transgenic seeds were sterilized as described in Example 4. The germinated seeds were sowed in a pot (8×8×8 cm) filled with mixture of organic soil and vermiculite (V:V=1:2). Three transgenic rice plants and 3 event null plants segregated from the hemizygous plants were planted in one pot, and rice plants of each event were planted in 6 pots. 24 pots planted with rice from 3 events were placed on one tray. The seedlings were grown under normal greenhouse condition and watered by modified IRRI solution for 18-21 days. When grown to 3-leaf stage, the seedlings were transferred into artificial chamber at 4° C. and stressed for 3-5 days until the leaves of 50% plants became curved. Then the plants were transferred into greenhouse to recover for 5-7 days, and the plants were scored for the degree of recovery. The following scoring system was used: more than half green stem=1, more than two third green leaf=1, less than two third but more than one third green leaf=0.5, less than one third green leaf=0.2, no green leaf or less than half green stem=0. The recovery degree was the sum of the score of the green tissues, and the data were statistically analyzed using Mixed Model. The events which showed significant better than controls (p<0.05) were considered as positive ones.

Survival rate (percentage of survived plants over the total plant number) was also used as a parameter for cold screening.

### Results:

### 1) DP0067 Transgenic Rice

In this experiment, 7 events were tested. After cold stressed for 4 days and recovered in greenhouse for 7 days, 6 events showed higher survival rates and 5 events showed higher recovery degrees, wherein, 4 events showed significantly higher recovery degrees. These results indicate that OsMYBI25-transgenic rice had enhanced cold tolerance than control at seedling stage.

TABLE 22
Enhanced cold tolerance of OsMYB125-transgenic rice plants at T₂ generation
under low temperature

| | Number of | | | Average | | |
|---|---|---|---|---|---|---|
| | survived | Number of | Survival rate | recovery | | |
| Event ID | plants | total plants | (%) | degree | p-value | p ≦ 0.05 |
| DP0067.05 | 12 | 18 | 66.67 | 1.25 | 0.0138 | Y |
| DP0067.05-Null | 7 | 18 | 38.89 | 0.75 | | |
| DP0067.06 | 16 | 18 | 88.89 | 1.22 | 0.0086 | Y |
| DP0067.06-Null | 8 | 18 | 44.44 | 0.44 | | |
| DP0067.08 | 15 | 18 | 83.33 | 1.47 | 0.0054 | Y |
| DP0067.07-Null | 7 | 18 | 38.89 | 0.47 | | |
| DP0067.09 | 17 | 18 | 94.44 | 1.06 | 0.3632 | |
| DP0067.09-Null | 15 | 18 | 83.33 | 1.28 | | |
| DP0067.10 | 16 | 17 | 94.12 | 1.22 | 0.0103 | Y |
| DP0067.10-Null | 10 | 18 | 55.56 | 0.61 | | |
| DP0067.12 | 14 | 18 | 77.78 | 0.94 | 0.2950 | |
| DP0067.12-Null | 15 | 18 | 83.33 | 1.39 | | |
| DP0067.13 | 14 | 17 | 82.35 | 1.17 | 0.2586 | |
| DP0067.13-Null | 13 | 18 | 72.22 | 0.89 | | |

### 2) DP0142 Transgenic Rice

Nine OsDN-CTPI-transgenic events were tested in cold tolerance assay. As shown in Table 23, 6 events showed higher survival rates and recovery degrees, and 2 events showed significantly higher recovery degrees. These results indicate that OsDN-CTP1-transgenic rice had enhanced cold tolerance than control at seedling stage.

TABLE 23
Enhanced cold tolerance of OsDN-CTP1-transgenic rice plants at T₂ generation
under low temperature

| | Number of | | | Average | | |
|---|---|---|---|---|---|---|
| | survived | Number of | Survival rate | recovery | | |
| Event ID | plants | total plants | (%) | degree | p-value | p ≦ 0.05 |
| DP0142.02 | 12 | 18 | 66.7 | 0.73 | 0.6539 | |
| DP0142.02-Null | 13 | 18 | 72.2 | 0.83 | | |
| DP0142.06 | 9 | 18 | 50.0 | 0.57 | 0.1311 | |
| DP0142.06-Null | 5 | 18 | 27.8 | 0.28 | | |
| DP0142.09 | 13 | 18 | 72.2 | 0.90 | 0.0492 | Y |
| DP0142.09-Null | 9 | 18 | 50.0 | 0.51 | | |
| DP0142.12 | 6 | 18 | 33.3 | 0.56 | 0.7055 | |
| DP0142.12-Null | 6 | 17 | 35.3 | 0.67 | | |
| DP0142.13 | 12 | 18 | 66.7 | 0.69 | 0.3062 | |
| DP0142.13-Null | 6 | 18 | 33.3 | 0.49 | | |
| DP0142.21 | 5 | 18 | 27.8 | 0.54 | 0.6614 | |
| DP0142.21-Null | 7 | 18 | 38.9 | 0.60 | | |
| DP0142.23 | 8 | 17 | 47.1 | 0.87 | 0.0854 | |
| DP0142.23-Null | 4 | 18 | 22.2 | 0.35 | | |
| DP0142.25 | 11 | 18 | 61.1 | 0.62 | 0.0312 | Y |
| DP0142.25-Null | 5 | 18 | 27.8 | 0.28 | | |
| DP0142.29 | 6 | 18 | 33.3 | 0.58 | 0.5946 | |
| DP0142.29-Null | 5 | 18 | 27.8 | 0.42 | | |

### Example 7

### Laboratory Paraquat Assays of Transgenic Rice Plants

Paraquat (1,1-dimethyl-4,4-bipyridinium dichloride), is a foliar-applied and non-selective bipyridinium herbicide, and it is one of the most widely used herbicides in the world, controlling weeds in a huge variety of crops like corn, rice, soybean etc. In plant cells, paraquat mainly targets chloroplasts by accepting electrons from photosystem I and then reacting with oxygen to produce superoxide and hydrogen peroxide, which cause photooxidative stress. Drought stress and cols stress usually leads to increased reactive oxygen species (ROS) in plants and sometimes, the drought and/or cold tolerance of plant is associated with enhanced antioxidative ability. Paraquat is a potent oxidative stress inducer; it greatly increases the ROS production and inhibits the regeneration of reducing equivalents and compounds necessary for the activity of the antioxidant system. The ROS generation is enhanced under abiotic stress conditions, and the plant responses range from tolerance to death depending on the stress intensity and its associated-ROS levels. Relative low level of paraquat can mimic the stress-associated ROS production and used as a stress tolerance marker in plant stress biology (Hasaneen M. N. A. (2012) Herbicide-Properties, Synthesis and Control of Weeds book). Therefore, the paraquat tolerance of the drought tolerant and cold toleranttransgenic rice plants was tested.

Paraquat Assay Methods:

Transgenic rice plants from 8-10 transgenic events of eachtransgenic rice line were tested by paraquat assay. Tissue-cultured Zhonghua 11 plants (ZH11-TC) and empty vector transgenic plants (DP0158) were used as controls. T₂ transgenic seeds were sterilized and germinated as describedin Example 4, and this assay was carried out in growth room with temperature at 28-30° C. and humidity ˜30%. The germinated seeds were placed in a tube with a hole at the bottom, and water cultured at 30° C. for 5 days till one-leaf and one-terminal bud stage. Uniform seedlings about 3.5-4 cm in height were selected for paraquattesting. Randomized block design was used in this experiment. There were five blocks, each of which has 16×12 holes. Each transgenic event was placed in one row (12 plants/event), and ZH11-TC and DP0158 seedlings were placed in 3 rows (3×12 plants) randomly in one block. Then the seedlings were treated with 0.8 μM paraquat solution for 7 days at 10 h day/14 h night, and the treated seedlings first encountered dark and took up the paraquat solution which was changed every two days. After treated for 7 days, the green seedlings were counted. Those seedlings that maintain green in whole without damage were considered asparaquat tolerant seedling; those with bleached leaves or stem were not considered asparaquat tolerant seedling.

Tolerant rate was used as a parameter for this trait screen, which is the percentage of plants which kept green and showed tolerant phenotype over the total plant number.

The data was analyzed at construct level (all transgenic plants compared with the control) and transgenic event level (different transgenic events compared with the control) using a statistic model of “Y˜seg+event (seg)+rep+error”, random effect of “rep”, Statistic Method of “SAS ProcGlimmix”.

### Paraquat Assay Results:

### 1) DP0008-Transgenic Rice

After paraquat solution treated, 252 of 600 OsDN-DTP2-transgenic seedlings (52%) kept green and showed tolerant phenotype, while 33 of 180 (18%) seedlings from ZH11-TC showed tolerant phenotype, and only 21 of 180 (12%) DP0158 seedlings showed tolerant phenotype. The tolerant rate of all screened OsDN-DTP2-transgenic seedlings was significantly greater than that of the ZH11-TC (p-value=0.0000) andDP0158 (p-value=0.0000) controls. These results indicate that theOsDN-DTP2transgenic seedlings exhibited enhanced paraquat tolerance compared to both controls of ZH11-TC and DP0158 seedlings at construct level.

Further analysis at transgenic event level indicates that 8 events had greater tolerant rates compared with ZH11-TC control, and all 10 events had greater tolerant rates than DP0158 control (Table 24). These results demonstrate that OsDN-DTP2-transgenic rice plants had enhanced paraquat tolerance compared to both controls of ZH11-TC and DP0158 rice plants at construct and transgenic event level at seedling stages. OsDN-DTP2functions in enhancingparaquat tolerance or antioxidative ability of transgenic plants.

Over-expression of OsDN-DTP2 gene enhanced the drought tolerance of transgenic plants; the cross-validations further confirmed that OsDN-DTP2 plays a role in enhancing drought tolerance in plant.

TABLE 24
Paraquat tolerance assay of OsDN-DTP2-transgenic rice plants at T₂ generation
at transgenic event level

| | Number of | Number of | | | | | |
|---|---|---|---|---|---|---|---|
| | tolerant | total | Tolerant rate | CK = ZH11-TC | | CK = DP0158 | |
| Event ID | seedlings | seedlings | (%) | p-value | p ≦ 0.05 | p-value | p ≦ 0.05 |
| DP0008.27 | 39 | 60 | 65 | 0.0000 | Y | 0.0000 | Y |
| DP0008.31 | 24 | 60 | 40 | 0.0014 | Y | 0.0000 | Y |
| DP0008.32 | 42 | 60 | 70 | 0.0000 | Y | 0.0000 | Y |
| DP0008.38 | 29 | 60 | 48 | 0.0000 | Y | 0.0000 | Y |
| DP0008.39 | 27 | 60 | 45 | 0.0002 | Y | 0.0000 | Y |
| DP0008.42 | 11 | 60 | 18 | 0.9999 | | 0.1965 | |
| DP0008.43 | 11 | 60 | 18 | 0.9999 | | 0.1965 | |
| DP0008.45 | 16 | 60 | 27 | 0.1726 | | 0.0085 | Y |
| DP0008.47 | 22 | 60 | 37 | 0.0055 | Y | 0.0000 | Y |
| DP0008.48 | 31 | 60 | 52 | 0.0000 | Y | 0.0000 | Y |
| ZH11-TC | 33 | 180 | 18 | | | | |
| DP0158 | 21 | 180 | 12 | | | | |

### 2) DP0055-Transgenic Rice

For OsGSTU35-transgenic rice, 305 of 600 transgenic seedlings (51%) kept green and showed tolerant phenotype after treated with 0.8 μM paraquat solutions for 7 days, while 17 of 180 (9%) seedlings from ZH11-TC showed tolerant phenotype and only 31 of 180 (17%) seedlings from DP0158 showed tolerant phenotype. The tolerant rate of OsGSTU35-transgenic seedlings was significantly higher than that of ZH11-TC (p-value=0.0000) and DP0158 (p-value=0.0000) controls. The OsGSTU35-transgenic seedlings grew better after treatment with 0.8 μM paraquat solutions compared to ZH11-TC and DP0158 seedlings. These results indicate that the OsGSTU35-transgenic seedling exhibited enhanced paraquat tolerant rate compared to both ZH11-TC and DP0158controls at construct level.

Further analysis at transgenic event level is displayed in Table 25. All of the ten transgenic events had significantly higher tolerant rate than either ZH11-TC or DP0158 controls, and the tolerant rates of 9 events were more than 40%. These results clearly show thatover-expression OsGSTU35 gene under CaMV 35S promoter increased the paraquat tolerance or antioxidative ability of the transgenic plants.

As described in Example 4, over-expression of OsGSTU35 gene increased the drought tolerance of rice plants. These cross-validations confirm that OsGSTU35 plays a role in increasing drought tolerance in plant.

TABLE 25
Paraquat tolerance assay of OsGSTU35-transgenic rice plants at T₂ generation at
transgenic event level

| | Number of | Number of | Tolerant rate | CK = ZH11-TC | | CK = DP0158 | |
|---|---|---|---|---|---|---|---|
| Event ID | tolerant | total | (%) | p-value | p ≦ 0.05 | p-value | p ≦ 0.05 |
| DP0055.01 | 40 | 60 | 67 | 0.0000 | Y | 0.0000 | Y |
| DP0055.03 | 27 | 60 | 45 | 0.0000 | Y | 0.0000 | Y |
| DP0055.05 | 50 | 60 | 83 | 0.0000 | Y | 0.0000 | Y |
| DP0055.06 | 29 | 60 | 48 | 0.0000 | Y | 0.0000 | Y |
| DP0055.07 | 25 | 60 | 42 | 0.0000 | Y | 0.0004 | Y |
| DP0055.08 | 22 | 60 | 37 | 0.0000 | Y | 0.0031 | Y |
| DP0055.09 | 32 | 60 | 53 | 0.0000 | Y | 0.0000 | Y |
| DP0055.17 | 24 | 60 | 40 | 0.0000 | Y | 0.0008 | Y |
| DP0055.18 | 27 | 60 | 45 | 0.0000 | Y | 0.0000 | Y |
| DP0055.19 | 29 | 60 | 48 | 0.0000 | Y | 0.0000 | Y |
| ZH11-TC | 17 | 180 | 9 | | | | |
| DP0158 | 31 | 180 | 17 | | | | |

### 3) DP0060-Transgenic Rice

After paraquat solution treated, 159 of 600 OsCML1-transgenic seedlings (27%) kept green and showed tolerant phenotype, whereas only 11 of 180 (6%) seedlings from ZH11-TC showed tolerant phenotype, and only 26 of 180 (14%) DP0158 seedlings showed tolerant phenotype. The tolerant rate of all screened OsCML1-transgenicseedlings was significantly greater than that of the ZH11-TC (p-value=0.0000) andDP0158 (p-value=0.0331) controls. The OsCML1-transgenic seedlings grew better than ZH11-TC and DP0158 seedlings. These results show that the OsCML1-transgenic seedlings exhibited enhanced paraquattolerance compared with both controls of ZH11-TC and DP0158 seedlings at construct level.

Further analysis at transgenic event level is illustrated in Table 26. Nine events had greater tolerant rates compared with ZH11-TC control, and 6 events had greater tolerant rates than DP0158 control.The tolerant rates of 4 events were significantly greater than that of both ZH11-TC and DP0158 controls. These results demonstrate that OsCML1-transgenic rice plants had enhanced paraquat tolerance compared to both controls of ZH11-TC and DP0158 rice plants at construct and transgenic event level at seedling stages.

Over-expression of OsCML1 gene enhanced the drought tolerance of transgenic plants; these cross-validations further confirmed that OsCML1 plays a role in enhancing drought tolerance in plant.

TABLE 26
Paraquat tolerance assay of OsCML1-transgenic rice plants at T₂ generation at
transgenic event level

| | Number of | Number of | | | | | |
|---|---|---|---|---|---|---|---|
| | tolerant | total | Tolerant rate | CK = ZH11-TC | | CK = DP0158 | |
| Event ID | seedlings | seedlings | (%) | p-value | p ≦ 0.05 | p-value | p ≦ 0.05 |
| DP0060.02 | 8 | 60 | 13 | 0.0846 | | 0.8315 | |
| DP0060.03 | 3 | 60 | 5 | 0.7519 | | 0.0685 | |
| DP0060.04 | 5 | 60 | 8 | 0.5537 | | 0.2320 | |
| DP0060.06 | 8 | 60 | 13 | 0.0846 | | 0.8315 | |
| DP0060.07 | 10 | 60 | 17 | 0.0188 | Y | 0.6780 | |
| DP0060.09 | 13 | 60 | 22 | 0.0017 | Y | 0.1966 | |
| DP0060.10 | 24 | 60 | 40 | 0.0000 | Y | 0.0001 | Y |
| DP0060.11 | 28 | 60 | 47 | 0.0000 | Y | 0.0000 | Y |
| DP0060.13 | 39 | 60 | 65 | 0.0000 | Y | 0.0000 | Y |
| DP0060.14 | 21 | 60 | 35 | 0.0000 | Y | 0.0013 | Y |
| ZH11-TC | 11 | 180 | 6 | | | | |
| DP0158 | 26 | 180 | 14 | | | | |

### 4) DP0062-Transgenic Rice

162 of 600 OsIMFA1a-transgenic seedlings (27%) kept green and showed tolerant phenotype after treated with paraquat solution, whereas only 21 of 180 (12%), and only 20 of 180 (11%) DP0158 seedlings showed tolerant phenotype. The tolerant rate of OsIMFA1a-transgenic plants was significantly higher than that of the ZH11-TC (p-value=0.0003) and DP0158 (p-value=0.0002) controls. The OsIMFA1a-transgenic seedlings grew better after paraquat solution treatment when compared to either ZH11-TC or DP0158 seedlings. These results indicate that the OsIMFA1a -transgenic seedlings had enhanced paraquat tolerant rate compared with both ZH11-TC and DP0158 controls at construct level.

The analysis at transgenic event level is displayed in Table 27. All of the ten events had greater tolerant rates than either ZH11-TC or DP0158 seedlings, which further demonstrates that OsIMPA1a-transgenic rice plants had enhanced paraquat tolerance at construct and transgenic event level at seedling stages. Over-expression of OsIMPA1a gene improved the paraquat tolerance of the transgenic plants. Over-expression of OsIMPA1a also increased the drought tolerance as described in Example 4. These cross-validations by two different assays clearly indicate the function of OsIMPA1a gene in increasing drought tolerance in plant.

TABLE 27
Paraquat tolerance assay of OsIMPA1a-transgenic rice plants at T₂ generation at
transgenic event level

| | Number of | Number of | | | | | |
|---|---|---|---|---|---|---|---|
| | tolerant | total | Tolerant rate | CK = ZH11-TC | | CK = DP0158 | |
| Event ID | seedlings | seedlings | (%) | p-value | p ≦ 0.05 | p-value | p ≦ 0.05 |
| DP0062.01 | 15 | 60 | 25 | 0.0170 | Y | 0.0124 | Y |
| DP0062.03 | 9 | 60 | 15 | 0.5025 | | 0.4281 | |
| DP0062.04 | 12 | 60 | 20 | 0.1134 | | 0.0883 | |
| DP0062.05 | 13 | 60 | 22 | 0.0626 | | 0.0475 | Y |
| DP0062.06 | 16 | 60 | 27 | 0.0085 | Y | 0.0061 | Y |
| DP0062.10 | 17 | 60 | 28 | 0.0042 | Y | 0.0029 | Y |
| DP0062.14 | 37 | 60 | 62 | 0.0000 | Y | 0.0000 | Y |
| DP0062.19 | 19 | 60 | 32 | 0.0009 | Y | 0.0006 | Y |
| DP0062.23 | 12 | 60 | 20 | 0.1134 | | 0.0883 | |
| DP0062.25 | 12 | 60 | 20 | 0.1134 | | 0.0883 | |
| ZH11-TC | 21 | 180 | 12 | | | | |
| DP0158 | 20 | 180 | 11 | | | | |

### 5) DP0067-Transgenic Rice

351 of 480OsMYB125-transgenic seedlings (73%) kept green and showed tolerant phenotype after treated with paraquat solutions, whereas 167 of 300 (56%) ZH11-TC seedlings showed tolerant phenotype, and 98 of 180 (54%) DP0158 seedlings showed tolerant phenotype. The tolerant rate of OsMYB125-transgenic seedlings was significantly higher than that of the ZH11-TC (p-value=0.0000) and DP0158 (p-value=0.0000) controls. The OsMYB125-transgenic seedlings grew better after paraquat solution treatment when compared to either ZH11-TC or DP0158 seedlings. These results demonstrate that the OsMYB125-transgenic seedlings exhibited enhanced paraquat tolerant rate compared to both of ZH11-TC and DP0158 controls at construct level.

Table 28 illustrates the analysis at event level. All of the 8 tested events had higher tolerant rates than either ZH11-TC or DP0158 control. 4 events had significantly higher tolerant rates. These results further demonstrate that over-expression of OsMYB125 gene can increase the paraquat tolerance or antioxidative activity of transgenic rice plants.

OsMYB125-transgenic rice exhibited drought tolerance and cold tolerance as illustrated in Example 4 and Example 6. These cross-validations confirm that over-expression of OsMYB125 gene can enhance drought tolerance and cold tolerance in plant which may be through enhancing antioxidative activity.

TABLE 28
Paraquat tolerance assay of OsMYB125-transgenic rice plants at T₂ generation at
transgenic event level

| | Number of | Number of | | | | | |
|---|---|---|---|---|---|---|---|
| | tolerant | total | Tolerant rate | CK = ZH11-TC | | CK = DP0158 | |
| Event ID | seedlings | seedlings | (%) | p-value | p ≦ 0.05 | p-value | p ≦ 0.05 |
| DP0067.01 | 51 | 60 | 85 | 0.0002 | Y | 0.0002 | Y |
| DP0067.02 | 39 | 60 | 65 | 0.1885 | | 0.1588 | |
| DP0067.03 | 42 | 60 | 70 | 0.0460 | Y | 0.0398 | Y |
| DP0067.04 | 36 | 60 | 60 | 0.5391 | | 0.4560 | |
| DP0067.07 | 55 | 60 | 92 | 0.0000 | Y | 0.0000 | Y |
| DP0067.08 | 51 | 60 | 85 | 0.0002 | Y | 0.0002 | Y |
| DP0067.09 | 38 | 60 | 63 | 0.2788 | | 0.2343 | |
| DP0067.12 | 39 | 60 | 65 | 0.1885 | | 0.1588 | |
| ZH11-TC | 167 | 300 | 56 | | | | |
| DP0158 | 98 | 180 | 54 | | | | |

### 6) DP0196-Transgenic Rice

After culturing the seedlings with paraquat solutions for 7days,313 of 600 OsBCS1L-transgenic seedlings (52%) kept green and showed tolerant phenotype, while only 35 of 180 (19%) ZH11-TC seedlings showed tolerant phenotype, and 51 of 180 (28%) DP0158 seedlings showed tolerant phenotype. The tolerant rate of OsBCS1L-transgenic seedlings was significantly higher than that of the ZH11-TC (p-value=0.0000) and DP0158 (p-value=0.0000) controls. The OsBSCL1-transgenic seedlings grew better after paraquat solution treatment when compared to either ZH11-TC or DP0158 seedlings. These results indicate that the OsBCS1L-transgenic seedlings exhibited enhanced paraquat tolerant rate compared to both ZH11-TC and DP0158 controls at construct level.

Further analysis at transgenic event level is shown in Table 29. Nine of ten tested transgenic events had significantly higher tolerant rates than either ZH11-TC or DP0158 control, which clearly demonstrates that OsBCS1L-transgenic rice plants had enhanced paraquat tolerance compared to both ZH11-TC and DP0158 control at construct and transgenic event level at seedling stages. OsBCS1L gene plays a role in the improvement of paraquat tolerance or antioxidative activity of transgenic plants.

TABLE 29
Paraquat tolerance assay of OsBCS1L-transgenic rice plants at T₂ generation
at transgenic event level

| | Number of | Number of | | | | | |
|---|---|---|---|---|---|---|---|
| | tolerant | total | Tolerant rate | CK = ZH11-TC | | CK = DP0158 | |
| Event ID | seedlings | seedlings | (%) | p-value | p ≦ 0.05 | p-value | p ≦ 0.05 |
| DP0196.02 | 43 | 60 | 72 | 0.0000 | Y | 0.0000 | Y |
| DP0196.04 | 38 | 60 | 63 | 0.0000 | Y | 0.0000 | Y |
| DP0196.05 | 27 | 60 | 45 | 0.0003 | Y | 0.0212 | Y |
| DP0196.06 | 29 | 60 | 48 | 0.0000 | Y | 0.0066 | Y |
| DP0196.07 | 36 | 60 | 60 | 0.0000 | Y | 0.0000 | Y |
| DP0196.09 | 33 | 60 | 55 | 0.0000 | Y | 0.0005 | Y |
| DP0196.12 | 14 | 60 | 23 | 0.5201 | | 0.4548 | |
| DP0196.13 | 31 | 60 | 52 | 0.0000 | Y | 0.0019 | Y |
| DP0196.14 | 28 | 60 | 47 | 0.0002 | Y | 0.0120 | Y |
| DP0196.17 | 34 | 60 | 57 | 0.0000 | Y | 0.0003 | Y |
| ZH11-TC | 35 | 180 | 19 | | | | |
| DP0158 | 51 | 180 | 28 | | | | |

### 7) DP0142-Transgenic Rice

406 of 600 OsDN-CTP1-transgenic seedlings (68%) kept green and showed tolerant phenotype, while 86 of 180 (48%) ZH11-TC seedlings showed tolerant phenotype, and 77 of 180 (43%) DP0158 seedlings showed tolerant phenotype. The tolerant rate of all tested OsDN-CTP1-transgenic seedlings was significantly higher than that of the ZH11-TC (p-value=0.0000) and DP0158 (p-value=0.0000) controls. These results indicate that the OsDN-CTP1-transgenic seedling had enhanced paraquat tolerant rate compared to either ZH11-TC or DP0158 control seedlings at construct level, and the OsDN-CTP1-transgenic seedlings grew better after treatment by 0.8 μM paraquat solutions compared to ZH11-TC and DP0158 seedlings.

The analysis at transgenic event level indicates that 9 of 10 tested transgenic events had higher tolerant rates compared to either ZH11-TC or DP0158 control (Table 30). 6 events had significantly higher tolerant rates than ZH11-TC control, and 9 events had significantly higher tolerant rates than DP0158 control. These results demonstrate that OsDN-CTP1-transgenic rice plants exhibited enhanced paraquat tolerance compared with both ZH11-TC and DP0158 controls at construct and transgenic event level at seedling stages. Over-expression of OsDN-CTP1 gene increased the paraquat tolerance or antioxidative activity of transgenic plants.

Over-expression of OsDN-CTP1 also increased the cold tolerance of transgenic rice plants; these cross-validations by two different assays indicate that OsDN-CTP1 may increase cold tolerance through increasing antioxidative activity of transgenic plants.

TABLE 30
Paraquat tolerance assay of OsDN-CTP1-transgenic rice plant at T₂ generation at
transgenic event level

| | Number of | Number of | | | | | |
|---|---|---|---|---|---|---|---|
| | tolerant | total | Tolerant rate | CK = ZH11-TC | | CK = DP0158 | |
| Event ID | seedlings | seedlings | (%) | p-value | p ≦ 0.05 | p-value | p ≦ 0.05 |
| DP0142.02 | 35 | 60 | 58 | 0.1609 | | 0.0409 | Y |
| DP0142.06 | 29 | 60 | 48 | 0.9409 | | 0.4542 | |
| DP0142.09 | 35 | 60 | 58 | 0.1609 | | 0.0409 | Y |
| DP0142.12 | 39 | 60 | 65 | 0.0246 | Y | 0.0044 | Y |
| DP0142.13 | 39 | 60 | 65 | 0.0246 | Y | 0.0044 | Y |
| DP0142.23 | 55 | 60 | 92 | 0.0000 | Y | 0.0000 | Y |
| DP0142.24 | 48 | 60 | 80 | 0.0000 | Y | 0.0000 | Y |
| DP0142.25 | 36 | 60 | 60 | 0.1058 | | 0.0244 | Y |
| DP0142.27 | 45 | 60 | 75 | 0.0007 | Y | 0.0000 | Y |
| DP0142.29 | 45 | 60 | 75 | 0.0007 | Y | 0.0000 | Y |
| ZH11-TC | 86 | 180 | 48 | | | | |
| DP0158 | 77 | 180 | 43 | | | | |

In summary, OsDN-DTP2, OsGSTU35, OsCML1, OsIMPA1a, OsMYB125, OsBCS1L,and OsDN-CTP1-transgenic rice demonstrated paraquat tolerance compared to both ZH11-TC and DP0158controls. Increased expression of OsDN-DTP2, OsGSTU35, OsCML1, OsIMPA1a, OsMYB125, OsBCS1L,and OsDN-CTP1 improved paraquat tolerance of transgenic plants.

### Example 8

### Transformation and Evaluation of Maize with Rice Drought Tolerance Genes

Maize plants can be transformed to over-express Oryza sativa drought tolerance genes or a corresponding homolog from maize, Arabidopsis, or other species. Expression of the gene in the maize transformation vector can be under control of a constitutive promoter such as the maize ubiquitin promoter (Christensen et al. (1989) Plant Mol. Biol. 12:619-632 and Christensen et al. (1992) Plant Mol. Biol. 18:675-689) or under control of another promoter, such as a stress-responsive promoter or a tissue-preferred promoter. The recombinant DNA construct can be introduced into maize cells by particle bombardment substantially as described in International Patent Publication WO 2009/006276. Alternatively, maize plants can be transformed with the recombinant DNA construct by Agrobacterium-mediated transformation substantially as described by Zhao et al. in Meth. Mol. Biol. 318:315-323 (2006) and in Zhao et al., Mol. Breed 8:323-333 (2001) and U.S. Pat. No. 5,981,840 issued Nov. 9, 1999. The Agrobacterium-mediated transformation process involves bacterium inoculation, co-cultivation, resting, selection and plant regeneration.

Progeny of the regenerated plants, such as T₁ plants, can be subjected to a soil-based drought stress. Using image analysis, plant area, volume, growth rate and color can be measured at multiple times before and during drought stress. Significant delay in wilting or leaf area reduction, a reduced yellow-color accumulation, and/or an increased growth rate during drought stress, relative to a control, will be considered evidence that the gene functions in maize to enhance drought tolerance.

### Example 9

### Transformation and Evaluation of Gaspe Flint Derived Maize Lines

As described in Example 8, maize plants can be transformed to over-express the rice drought tolerance genes, or corresponding homologs from another species. In certain circumstances, recipient plant cells can be from a uniform maize line having a short life cycle (“fast cycling”), a reduced size, and high transformation potential, and are disclosed in Tomes et al. U.S. Pat. No. 7,928,287.

The population of transgenic (T₀) plants resulting from the transformed maize embryos can be grown in a controlled greenhouse environment using a modified randomized block design to reduce or eliminate environmental error. For example, a group of 30 plants, comprising 24 transformed experimental plants and 6 control plants (collectively, a “replicate group”), are placed in pots which are arranged in an array (a.k.a. a replicate group or block) on a table located inside a greenhouse. Each plant, control or experimental, is randomly assigned to a location with the block which is mapped to a unique, physical greenhouse location as well as to the replicate group. Multiple replicate groups of 30 plants each may be grown in the same greenhouse in a single experiment. The layout (arrangement) of the replicate groups should be determined to minimize space requirements as well as environmental effects within the greenhouse. Such a layout may be referred to as a compressed greenhouse layout.

Each plant in the event population is identified and tracked throughout the evaluation process, and the data gathered from that plant are automatically associated with that plant so that the gathered data can be associated with the transgene carried by the plant. For example, each plant container can have a machine readable label (such as a Universal Product Code (UPC) bar code) which includes information about the plant identity, which in turn is correlated to a greenhouse location so that data obtained from the plant can be automatically associated with that plant.

Alternatively any efficient, machine readable, plant identification system can be used, such as two-dimensional matrix codes or even radio frequency identification tags (RFID) in which the data is received and interpreted by a radio frequency receiver/processor (U.S. Pat. Nos. 7,403,855 and 7,702,462).

Each greenhouse plant in the T₀ event population, including any control plants, is analyzed for agronomic characteristics of interest, and the agronomic data for each plant are recorded or stored in a manner so as to be associated with the identifying data for that plant. Confirmation of a phenotype (gene effect) can be accomplished in the T₁ generation with a similar experimental design to that described above.

### Example 10

### Laboratory Drought Screening of Rice Drought Tolerance Genes in Arabidopsis

To understand whether rice drought tolerance genes can improve dicot plants' drought tolerance, or other traits, the rice drought tolerance gene over-expression vectors were transformed into Arabidopsis (Columbia) using floral dip method by Agrobacterium mediated transformation procedure and transgenic plants were identified (Clough, S. T. and Bent, A. F. (1998) The Plant Journal 16, 735-743; Zhang, X. et al. (2006) Nature Protocols 1: 641-646).

A 16.8-kb T-DNA based binary vector which is called pBC-yellow was used in this experiment. This vector contains the RD29a promoter driving expression of the gene for ZS-Yellow, which confers yellow fluorescence to transformed seed. The rice tolerance genes were cloned as described in Example 1, and constructed in the Gateway vector. Then using the INVITROGEN™ GATEWAY® technology, an LR Recombination Reaction was performed on the entry clone containing the directionally cloned PCR product and the pBC-yellow vector, and the over-expression vectors were obtained.

T₂ seeds were used for lab drought assay. Arabidopsis drought screening is a soil-based water withdrawal assay performed in a growth chamber with conditions of light intensity 145 μMol, temperature 22° C. day/20° C. night and humidity ˜60%. The transgenic seeds were sorted by Copas (Complex Object Parametric Analyzer and Sorter, a seed sorter), and were stratified by putting in 0.1% agarose solution, and placing at 4° C. for 3 days. Wild-type Arabidopsis were used as control and stratified as above. 36 plants each for over-expression transgenic Arabidopsis and wild-type were planted equidistantly and alternatively to each other in a zig-zag fashion. The soil composition was 3 parts peat moss, 2 parts vermiculite and 1 part perlite. Apart from these, fertilizers and fungicides were added to the soil in the following concentrations: NPK (Nitrogen, Phosphorus, Potassium)—1 gm/kg soil, Micronutrients—0.5 gm/kg soil, Fungicide—0.5 gm/kg soil. Plants were thinned to 9 plants per pot (72 plants per flat), and were well watered for the first 12 days, then saturated with 1 L of deionized water for 30 min with excess water drained off completely. The plants were imaged between days 28 and 36 after germination using an imaging device and data were analyzed. The flats were rotated each day from the second day after sowing till the last day of imaging. The files generated in the imaging device were converted into XLS files and put in a Stan's format and sent to ESL for generating Stan's score for the experimental lines. Rate of decay or wilting under drought conditions is used as tested parameter. The cut-off Score=1.5.

1. An isolated polynucleotide, comprising: (a) a polynucleotide with nucleotide sequence of at least 85% sequence identity to SEQ ID NO: 3, 6, 9, 12, 15 or 18; (b) a polynucleotide with nucleotide sequence of at least 85% sequence identity to SEQ ID NO: 4, 7, 10, 13, 16 or 19;(c) a polynucleotide encoding a polypeptide comprising an amino acid sequence of at least 90% sequence identity to the full length SEQ ID NO: 5, 8, 11, 14, 17 or 20; or (d) the full complement of the nucleotide sequence of (a), (b) or (c), wherein the polynucleotide is operably linked to a heterologous regulatory element.

2. The isolated polynucleotide of claim 1, wherein the polynucleotide comprises the nucleotide sequence of SEQ ID NO: 3, SEQ ID NO: 4, SEQ ID NO: 6, SEQ ID NO: 7, SEQ ID NO: 9, SEQ ID NO: 10, SEQ ID NO: 12, SEQ ID NO: 13, SEQ ID NO: 15, SEQ ID NO: 16, SEQ ID NO: 18 or SEQ ID NO: 19.

3. The isolated polynucleotide of claim 1, wherein the isolated polynucleotide encoded polypeptide comprises the amino acid sequence of SEQ ID NO: 5, SEQ ID NO: 8, SEQ ID NO: 11, SEQ ID NO: 14, SEQ ID NO: 17 or SEQ ID NO: 20.

4. A vector comprising the polynucleotide of claim 1.

5. A recombinant DNA construct comprising the isolated polynucleotide of claim 1.

6. A plant or seed comprising a polynucleotide encoding a polypeptide comprising an amino acid sequence of at least 90% sequence identity to SEQ ID NO: 5, 8, 11, 14, 17 or 20, wherein the polynucleotide is operably linked to a regulatory element that increases the expression level of the polynucleotide compared to a control plant.

7. The plant of claim 6, wherein said plant exhibits improved drought tolerance when compared to the control plant.

8. (canceled)

9. (canceled)

10. (canceled)

11. (canceled)

12. (canceled)

13. (canceled)

14. The plant or seed comprising a genetic modification, wherein the genetic modification that results in reduced expression of a polynucleotide encoding a polypeptide with amino acid sequence of at least 90% sequence identity to SEQ ID NO: 23.

15. The plant or seed of claim 14, wherein the genetic modification is a modification of a regulatory sequence of the polynucleotide.

16. The plant of claim 14, wherein the genetic modification is by a RNAi suppression.

17. The plant of claim 14, wherein the genetic modification is a site-specific.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. The plant of claim 6, wherein said plant is selected from the group consisting of rice, maize, soybean, sunflower, sorghum, canola, wheat, alfalfa, cotton, barley, millet, sugar cane and switchgrass.

31. A method of increasing drought tolerance in a plant, comprising growing the plant of claim 6 and exposing the plant to drought stress.

32. A method of increasing drought tolerance in a plant, comprising growing the plant of claim 14 and exposing the plant to drought stress.

33. The method of claim 31, wherein the plant is maize or rice.

34. The method of claim 31, wherein the plant is maize or rice.

35. (canceled)

36. (canceled)

37. (canceled)

38. The plant of claim 6, wherein the regulatory sequence is a promoter.

39. The plant of claim 6, wherein the regulatory sequence is an enhancer element.

40. The plant of claim 6 is drought tolerant maize.

41. The plant of claim 6 is drought tolerant rice.


SEQUENCE LISTING (61 sequences)

SEQ ID NO: 1 - DNA, 10952 bp, Artificial Sequence
  vector DP0005
gaattctctagtcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaacgcggccgcttcagttgtggcccagcttggaggtcgactcgcgaggatcctctagtcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaacgcggccgcttcagttgtggcccagcttggagggggcggcgtcgcagtagcggcccacggcggcctcgtactgcttgtagcacttgcccttctccacctcctccaggatctcgatgcggtggtcctcgaagtggaagccgggcatcttcagggcggaggcgggcttcttggagcggtaggtggtgtgcaggtggcaggtcaggtggcgaccgccggggcactccagggccatcagggactggccgcgcagcacgccgtccacctcgtacacgatctcggtggagggctcccagcggccggccttgttctgcatcacggggccgtcggcggggaagttgttgcccaggatcttcaccttgtacaccaggcagtcgccgtccagggaggtgtcctggtgggcggtcaggaagccgccgtcctcgtaggtggtggtgcgctcccaggtgaagccctcggggagggactgcttgaagtagtcggggatgccggacacgtacttgatgaaggccttggagccgtacatgcaggaggtggacaggatgtggaaggcgaagggcagggggccgccctcgatcacctcgatcttcatctcctgggtgccctccagggggttgccctcgcccttgccggtgcacttgaagtagtggccgttcacggtgccctcgatggtggtcctgaagggcatggtcttcttcagcaaagaggccatggtggcgaccggtaccagatctctgcagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgccaagctgctctaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgttactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgacagatccggtcggcatctactctatttctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagtcgtggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggttgtagaacagcgggcagttcggtttcaggcaggtcttgcaacgtgacaccctgtgcacggcgggagatgcaataggtcaggctctcgctaaactccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacg

SEQ ID NO: 2 - DNA, 1921 bp, Artificial Sequence
  The nucleotide sequence of DsRed expression cassette
cgaagctggccgctctagaactagtggatctcgatgtgtagtctacgagaagggttaaccgtctcttcgtgagaataaccgtggcctaaaaataagccgatgaggataaataaaatgtggtggtacagtacttcaagaggtttactcatcaagaggatgcttttccgatgagctctagtagtacatcggacctcacatacctccattgtggtgaaatattttgtgctcatttagtgatgggtaaattttgtttatgtcactctaggttttgacatttcagttttgccactcttaggttttgacaaataatttccattccgcggcaaaagcaaaacaattttattttacttttaccactcttagctttcacaatgtatcacaaatgccactctagaaattctgtttatgccacagaatgtgaaaaaaaacactcacttatttgaagccaaggtgttcatggcatggaaatgtgacataaagtaacgttcgtgtataagaaaaaattgtactcctcgtaacaagagacggaaacatcatgagacaatcgcgtttggaaggctttgcatcacctttggatgatgcgcatgaatggagtcgtctgcttgctagccttcgcctaccgcccactgagtccgggcggcaactaccatcggcgaacgacccagctgacctctaccgaccggacttgaatgcgctaccttcgtcagcgacgatggccgcgtacgctggcgacgtgcccccgcatgcatggcggcacatggcgagctcagaccgtgcgtggctggctacaaatacgtaccccgtgagtgccctagctagaaacttacacctgcaactgcgagagcgagcgtgtgagtgtagccgagtagatcctcgccaccatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgacgaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtagcggcccatggatattcgaacgcgtaggtaccacatggttaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaatgcggcc

SEQ ID NO: 3 - DNA, 2767 bp, Oryza sativa
gatccgattcaacacaaagaggcaacatttttagcaacagacatggctttccaccaaagatcaattagcttgccttccaggcctatctccaaagttgaagaggagctgcacagcattgaggcatggatctcttcaccctccctgaccatcgagacaatctctgatggtttcaggaggcttggggacatctacagctccattgaggagatcatgtgcctgcctagcaaccaagtttgctcatccgagcagaggaggttgttggatggagagatggaatgctcccttgagctgctggatctctgcaacgctatgaacgaggtcttcaccgagttgaaggccatcatccaagatctgcaagtgtctctcaggaaaggagatggtgcagttcttcaagccaagatccagtcatacatccgcttggtgaagaaggcaaagaaacactccaagaagactctgacgaaggttgtctcagacaaggaggactgcaggatagtcaagctgttgagcgaggctagggagatcactacctctctatttgagtcaacaacgcacctcttgtcgaagcaaattgctacgccaaaattgtctctcatttctaaggcattccagaagaaaaacccagtgatttgcaatgaggaccagttgcaggtgttagagtgctccatcagagatcttgaggctggagcaggacttctgttcaggagattggtccagagcagggttactctcctcaacattcttagctcatagatgctcctcaagatctgtcactcctaaaacctgcgattggcgtccaccttttaaaggatttgctgatccttaccttgtatatgtcatagatttatagtgtacagaaaaaaagttatacatgaaagaaacagaaattttgatctaattgtgcgctcaatcctcatgatgtgattatgcaacaagatgccaaaagccgttgtgatgaatataatttgcgcaagccggcacatgaattatcaaatatatgtgccgcgttagcaattctactttcatttcttttatattttatagtcaaattgataatgatgtgccatagggctgtgaaatgcccatgtgggccatgtaactctgatgctgtttgttgcctcactagcaagcaaaggatgcatgtactgtggatcttgctgctgcagccgaaacagaccagctccaatacacgaggttaagcgtgtaagcagcatggattgcacttattagaacacaagttgaaactaacaagagcattaataattagataacacgcatgtcaactataatactctggtatcacgctattaaaataatcccttgagagcatgcaattattccaagaaccaccggtagagtgaactaacctgctgattcttgctgccgataattgggacatgacaatgcgatagctcacttggaagatagacggcaatgcattaaaacattgaacaacaaagagacttgcaacagccagatctcaaaaccatgacagacagcatcagggagttgaactgccagtattctatttgtctaccatccaattgatgtagtgtcttgcacatcctctgtataaataggtctaaccacaaagctagacacatcaaaccaagactttcctctccttctcagctctcagactcaacagagagacagagcttagcaacacacatggctttccaccaaagatcagttagcttgcgttccaggcctctctccaaagttgaagaggagctgcacagcgtagaggcatgcatctcttcaccctccctgaccatcgaggcaatctccgatggtctgagggggctcggggacatctactgctcaattgaggagatcatgtgcctgcctagcaaccaagtttgctcaccacagcaaaggaagttgttggatggagagatggaatgctcccttgagctactggatatgtgcaacactatgagcgaggtcttcaccgagttgaaggccatcatccaagatctgcaagtgtctctcagaaaaggagatgatgcagttcttcaagccaagatccagtcatacatccgcttggtgaagaaggcaaagaaacattccaagaagactctgaagaaggttgtctcgaacaaggaggactgcaggatagtcaagctattgagagaggctagagagattactacctctctattcgagtcaactacacacctcttgtcgaagcaaattgctatgcctaaattgtctctcatctccaaggcattccagaagaaaatcccagtgatttgcaatgaggagcagttgcaggtgttagagtgctgcatcagagatcttgaggctggagcagggcttctgttcaggagattggtccaaagcagggttactctcctgaacattcttagctcatagatactcaagatctgtcactcttaataccctgtgattggcatccgccttttaaaggatttgctgatccttccatctgtatatgccatagaatagaattactgtacaggaaaataaaatatacatgaaagagatacaaagttttgatctaattcttgccgtgtgctcaggcttcacatattgctgagatacaagatgtgattacgcaatgtgctgtcagtattattgcctttgggattaatatacaacggacaatccaacaaatgagttatgaaatatatgtgccatgctagtgatattattttcatttcttttgtatttttacagtcaaattaatccatagggatatacatgctatacctctagacatgaggattgcagacaaatacctcga

SEQ ID NO: 4 - DNA, 708 bp, Oryza sativa
atggctttccaccaaagatcaattagcttgccttccaggcctatctccaaagttgaagaggagctgcacagcattgaggcatggatctcttcaccctccctgaccatcgagacaatctctgatggtttcaggaggcttggggacatctacagctccattgaggagatcatgtgcctgcctagcaaccaagtttgctcatccgagcagaggaggttgttggatggagagatggaatgctcccttgagctgctggatctctgcaacgctatgaacgaggtcttcaccgagttgaaggccatcatccaagatctgcaagtgtctctcaggaaaggagatggtgcagttcttcaagccaagatccagtcatacatccgcttggtgaagaaggcaaagaaacactccaagaagactctgacgaaggttgtctcagacaaggaggactgcaggatagtcaagctgttgagcgaggctagggagatcactacctctctatttgagtcaacaacgcacctcttgtcgaagcaaattgctacgccaaaattgtctctcatttctaaggcattccagaagaaaaacccagtgatttgcaatgaggaccagttgcaggtgttagagtgctccatcagagatcttgaggctggagcaggacttctgttcaggagattggtccagagcagggttactctcctcaacattcttagctcatag

SEQ ID NO: 5 - protein, 235 aa, Oryza sativa
MAFHQRSISLPSRPISKVEEELHSIEAWISSPSLTIETISDGFRRLGDIYSSIEEIMCLPSNQVCSSEQRRLLDGEMECSLELLDLCNAMNEVFTELKAIIQDLQVSLRKGDGAVLQAKIQSYIRLVKKAKKHSKKTLTKVVSDKEDCRIVKLLSEAREITTSLFESTTHLLSKQIATPKLSLISKAFQKKNPVICNEDQLQVLECSIRDLEAGAGLLFRRLVQSRVTLLNILSS

SEQ ID NO: 6 - DNA, 757 bp, Oryza sativa
acgatgggtgaaagggtgaagctcatcggtgctttcgccagtgcatacggccaccgcgcagaggtggcgcttcgcctgaaaggcgtgcgatacgagctcatcctggaagacctccgcaacaagagcgacctgctgctcaaccacaaccccgtccacaagctcgtccccgtcctcctccatggcgaccgctccttgagcgagtccctcgtcatcctcgagtacatcgacgagagcttccatggtccacccatcctcccaaccgatccgtacgatcgagccgtggcgcgtttctgggcgcagttcatcgatcagaagtttggtaggttcaatttctggatcccgttcgtgcaaatggagggcaacatgcaggattgtttcgtgagggaagcaaaggagaatctggcgcttcttgaagggcagctcaaggggaggagattcttcggaggcgacgccatcgggttcttggacatagcagcgtgcttgatagctcactggcttggtgcgttcgaggaggtatgtggggtgaccttggccacggatgaggagttccctgctttgtgcgagtggaggagacgctacgtcaacgatgaggccgtgaagccgtgcctgccgaatagggacgaactcgttgcgtattaccgtgaacgcaaggagatgatcaaagccgccggaaggcagcacaaatgattccaacgtagttgtatgcatgagaaataaatatatgtccatgggaatggaataagttactatttgattc

SEQ ID NO: 7 - DNA, 684 bp, Oryza sativa
atgggtgaaagggtgaagctcatcggtgctttcgccagtgcatacggccaccgcgcagaggtggcgcttcgcctgaaaggcgtgcgatacgagctcatcctggaagacctccgcaacaagagcgacctgctgctcaaccacaaccccgtccacaagctcgtccccgtcctcctccatggcgaccgctccttgagcgagtccctcgtcatcctcgagtacatcgacgagagcttccatggtccacccatcctcccaaccgatccgtacgatcgagccgtggcgcgtttctgggcgcagttcatcgatcagaagtttggtaggttcaatttctggatcccgttcgtgcaaatggagggcaacatgcaggattgtttcgtgagggaagcaaaggagaatctggcgcttcttgaagggcagctcaaggggaggagattcttcggaggcgacgccatcgggttcttggacatagcagcgtgcttgatagctcactggcttggtgcgttcgaggaggtatgtggggtgaccttggccacggatgaggagttccctgctttgtgcgagtggaggagacgctacgtcaacgatgaggccgtgaagccgtgcctgccgaatagggacgaactcgttgcgtattaccgtgaacgcaaggagatgatcaaagccgccggaaggcagcacaaatga

SEQ ID NO: 8 - protein, 227 aa, Oryza sativa
MGERVKLIGAFASAYGHRAEVALRLKGVRYELILEDLRNKSDLLLNHNPVHKLVPVLLHGDRSLSESLVILEYIDESFHGPPILPTDPYDRAVARFWAQFIDQKFGRFNFWIPFVQMEGNMQDCFVREAKENLALLEGQLKGRRFFGGDAIGFLDIAACLIAHWLGAFEEVCGVTLATDEEFPALCEWRRRYVNDEAVKPCLPNRDELVAYYRERKEMIKAAGRQHK

SEQ ID NO: 9 - DNA, 647 bp, Oryza sativa
tctcccattcgagcgagatgaagctctccatccagtcattcgcccgcaagctctccctcccgtcgccgaagcggacgtggagcagcggcggcggaagcagtaagagggatggtggcatgtccaagaacgggagcggcgtgaagcgggccatctcccgcagcgaggcgtcgtcgttcgcgtcggcgtcgtcggagtcggagtcgtcctcggacgacgcgctgatggcgaggtcgacaccgaggtcggtgctccccgcggagatctcgcggcgggagctggaggccgtgctccggcggctcgggcacggggagcccgacgacgaggagctggacgccgtcgcggccatcgccgccgaggccgaggcgggcggcggggaggacgagctgatggaggcgttcaaggtgttcgacgccgacggcgacggccgcatcaccgccgaggagctccgcggcgtcatggtcgccatcctcggcggcgacggcgacggctgcagcctcgacgactgccgccgcatgatcggcggcgtcgacgccgacggcgacggcttcgtcgggttccaggacttcgcccgcatgatgatggccgccaccgccaccgccacggcgacggcggacggcccgagatcgtggtgatccattcctccgttc

SEQ ID NO: 10 - DNA, 615 bp, Oryza sativa
atgaagctctccatccagtcattcgcccgcaagctctccctcccgtcgccgaagcggacgtggagcagcggcggcggaagcagtaagagggatggtggcatgtccaagaacgggagcggcgtgaagcgggccatctcccgcagcgaggcgtcgtcgttcgcgtcggcgtcgtcggagtcggagtcgtcctcggacgacgcgctgatggcgaggtcgacaccgaggtcggtgctccccgcggagatctcgcggcgggagctggaggccgtgctccggcggctcgggcacggggagcccgacgacgaggagctggacgccgtcgcggccatcgccgccgaggccgaggcgggcggcggggaggacgagctgatggaggcgttcaaggtgttcgacgccgacggcgacggccgcatcaccgccgaggagctccgcggcgtcatggtcgccatcctcggcggcgacggcgacggctgcagcctcgacgactgccgccgcatgatcggcggcgtcgacgccgacggcgacggcttcgtcgggttccaggacttcgcccgcatgatgatggccgccaccgccaccgccacggcgacggcggacggcccgagatcgtggtga

SEQ ID NO: 11 - protein, 204 aa, Oryza sativa
MKLSIQSFARKLSLPSPKRTWSSGGGSSKRDGGMSKNGSGVKRAISRSEASSFASASSESESSSDDALMARSTPRSVLPAEISRRELEAVLRRLGHGEPDDEELDAVAAIAAEAEAGGGEDELMEAFKVFDADGDGRITAEELRGVMVAILGGDGDGCSLDDCRRMIGGVDADGDGFVGFQDFARMMMAATATATATADGPRSW

SEQ ID NO: 12 - DNA, 751 bp, Oryza sativa
gcacgaggctggggatgacatgcagactcagtgtgtcatcgatcatcaagctcttccatgtctcttgaacctcttgaccaacaatcataagaaaagcatcaagaaagaagcatgctggactatctcaaacatcactgctggcaatagggaacagattcaggctgtgatcaatgcaaacataattgcccctctagtacatctgctgcaaactgctgaatttgacatcaagaaagaggctgcgtgggcaatctcaaatgccacttctggtggaacacatgatcagattaagtaccttgttgcccagggttgcatcaagccactctgtgatctgcttgtttgcccagatcccaggatcgtgacagtttgcttggaaggtcttgagaacatcttgaaggttggagaggcagaaaagaaccttggggcaggggatgtcaattcctatgctcagatgattgatgatgctgagggactggagaagattgagaaccttcagagccatgacaacactgaaatatatgagaaggcagttaaaatgctcgagtcctactggttggaggaggaagatgatgccatgccctcaggtgacaacgctcaaaacggcttcaactttggaaaccagcagcccaatgttccatcgggtggattcaactttggctgaagatacctatctggaatgatgtaccactgttccttagctacttgcttggggctagtcagagttgggggagtcttgtcgttggagtcttggttg

SEQ ID NO: 13 - DNA, 639 bp, Oryza sativa
atgcagactcagtgtgtcatcgatcatcaagctcttccatgtctcttgaacctcttgaccaacaatcataagaaaagcatcaagaaagaagcatgctggactatctcaaacatcactgctggcaatagggaacagattcaggctgtgatcaatgcaaacataattgcccctctagtacatctgctgcaaactgctgaatttgacatcaagaaagaggctgcgtgggcaatctcaaatgccacttctggtggaacacatgatcagattaagtaccttgttgcccagggttgcatcaagccactctgtgatctgcttgtttgcccagatcccaggatcgtgacagtttgcttggaaggtcttgagaacatcttgaaggttggagaggcagaaaagaaccttggggcaggggatgtcaattcctatgctcagatgattgatgatgctgagggactggagaagattgagaaccttcagagccatgacaacactgaaatatatgagaaggcagttaaaatgctcgagtcctactggttggaggaggaagatgatgccatgccctcaggtgacaacgctcaaaacggcttcaactttggaaaccagcagcccaatgttccatcgggtggattcaactttggctga

SEQ ID NO: 14 - protein, 212 aa, Oryza sativa
MQTQCVIDHQALPCLLNLLTNNHKKSIKKEACWTISNITAGNREQIQAVINANIIAPLVHLLQTAEFDIKKEAAWAISNATSGGTHDQIKYLVAQGCIKPLCDLLVCPDPRIVTVCLEGLENILKVGEAEKNLGAGDVNSYAQMIDDAEGLEKIENLQSHDNTEIYEKAVKMLESYWLEEEDDAMPSGDNAQNGFNFGNQQPNVPSGGFNFG

SEQ ID NO: 15 - DNA, 837 bp, Oryza sativa
atgatgtaccatgcaaagaagttctctgtaccctttggaccgcagagtacacagagtaacgagcatatgagtaatattggagcttttggcgggtcaaacatgggcagccctgctaatcctgcagggagtgggaaacaacggctacgttggacctcagatctccataaccgctttgtggatgctattgctcagcttggtggacctgatagagcaacacctaaaggggttctcactgtaatgggtgttcctgggatcacaatttatcatgtgaagagccatttgcagaaatatcgccttgcaaagtacataccagaatctcctgctgaaggctcaaaagacgaaaagaaggattctagcgattccctctctaacacagattctgcaccaggaatgcaaatcaatgaagctttgaagatgcaaatggaggtccagaagcgactccatgaacaacttgaggtgcaaaggcagctgcagctgagaattgaagcacaagggaagtacttgcagatgatcatagaggagcagcaaaagctcggtggatcactcaaagcttgtgaggagcagaagctaccgcattcaccaccaagcttagatgactacccagatagcatgcagccatctccaaagaaacccaagatggacaacctgtcacctgattcggtacgggatgtgacacagtcagattttgaatcccatttgattggtccttgggatcaagaggctgcattccgagtggatgaatttaaagctgaccctggtctgaacaaatcataaagcaaaacctcactcatcggaaattcttgatccaagatgttaacctccactgcgggccgatcg

SEQ ID NO: 16 - DNA, 774 bp, Oryza sativa
atgatgtaccatgcaaagaagttctctgtaccctttggaccgcagagtacacagagtaacgagcatatgagtaatattggagcttttggcgggtcaaacatgggcagccctgctaatcctgcagggagtgggaaacaacggctacgttggacctcagatctccataaccgctttgtggatgctattgctcagcttggtggacctgatagagcaacacctaaaggggttctcactgtaatgggtgttcctgggatcacaatttatcatgtgaagagccatttgcagaaatatcgccttgcaaagtacataccagaatctcctgctgaaggctcaaaagacgaaaagaaggattctagcgattccctctctaacacagattctgcaccaggaatgcaaatcaatgaagctttgaagatgcaaatggaggtccagaagcgactccatgaacaacttgaggtgcaaaggcagctgcagctgagaattgaagcacaagggaagtacttgcagatgatcatagaggagcagcaaaagctcggtggatcactcaaagcttgtgaggagcagaagctaccgcattcaccaccaagcttagatgactacccagatagcatgcagccatctccaaagaaacccaagatggacaacctgtcacctgattcggtacgggatgtgacacagtcagattttgaatcccatttgattggtccttgggatcaagaggctgcattccgagtggatgaatttaaagctgaccctggtctgaacaaatcataa

SEQ ID NO: 17 - protein, 257 aa, Oryza sativa
MMYHAKKFSVPFGPQSTQSNEHMSNIGAFGGSNMGSPANPAGSGKQRLRWTSDLHNRFVDAIAQLGGPDRATPKGVLTVMGVPGITIYHVKSHLQKYRLAKYIPESPAEGSKDEKKDSSDSLSNTDSAPGMQINEALKMQMEVQKRLHEQLEVQRQLQLRIEAQGKYLQMIIEEQQKLGGSLKACEEQKLPHSPPSLDDYPDSMQPSPKKPKMDNLSPDSVRDVTQSDFESHLIGPWDQEAAFRVDEFKADPGLNKS

SEQ ID NO: 18 - DNA, 686 bp, Oryza sativa
cttgtgttactaataatctttgaggggaggcaattaatggaccacctgacaaaggagcagatcgccgagttccgggaggcattcaacctgttcgacaaagatggagacgggacgatcacgagcaaggagcttgggacggtgatggggtcgctggggcagtcgccgacggaggcggagctgaagaagatggtggaggaggtggacgcggacggcagcggcagcatcgagttcgaggagttcctgggcctcctcgcccgcaagcttcgcgacaccggcgccgaggacgacatccgcgacgccttccgcgtcttcgacaaggaccagaacggcttcatcacccccgacgagctccgccacgtcatggccaacctcagcgaccccctctccgacgacgagctcgccgacatgctccacgaggccgactccgacggcgacggccagatcaactacaacgagttcctcaaggtcatgatggcaaagcgaaggcagaatatgatggagggacatggaagtggaggccatcggtcaagtaactcccacaagaaatccggctgctgcggcccgaattcctcatgtaccatcctctgaaaaagatgtaggtttcaggtttgcaactgttctgatgaggattgtatagttcagagttttttttttgtcacctcaatttctggttacacttgttctgg

SEQ ID NO: 19 - DNA, 552 bp, Oryza sativa
atggaccacctgacaaaggagcagatcgccgagttccgggaggcattcaacctgttcgacaaagatggagacgggacgatcacgagcaaggagcttgggacggtgatggggtcgctggggcagtcgccgacggaggcggagctgaagaagatggtggaggaggtggacgcggacggcagcggcagcatcgagttcgaggagttcctgggcctcctcgcccgcaagcttcgcgacaccggcgccgaggacgacatccgcgacgccttccgcgtcttcgacaaggaccagaacggcttcatcacccccgacgagctccgccacgtcatggccaacctcagcgaccccctctccgacgacgagctcgccgacatgctccacgaggccgactccgacggcgacggccagatcaactacaacgagttcctcaaggtcatgatggcaaagcgaaggcagaatatgatggagggacatggaagtggaggccatcggtcaagtaactcccacaagaaatccggctgctgcggcccgaattcctcatgtaccatcctctga

SEQ ID NO: 20 - protein, 183 aa, Oryza sativa
MDHLTKEQIAEFREAFNLFDKDGDGTITSKELGTVMGSLGQSPTEAELKKMVEEVDADGSGSIEFEEFLGLLARKLRDTGAEDDIRDAFRVFDKDQNGFITPDELRHVMANLSDPLSDDELADMLHEADSDGDGQINYNEFLKVMMAKRRQNMMEGHGSGGHRSSNSHKKSGCCGPNSSCTIL

SEQ ID NO: 21 - DNA, 1592 bp, Oryza sativa
ctcaccctccccattcaacactactgtttcataccattaccaacaacaaagaggaagagaagttcatcaaaagaagaacaagagaggagccagagcttgctcaccatggcgtcctacgacaaggccatcgagtcatacaagaaggccatcacaaccgctgcatccgttgcagcgtctgtgatgctggtccgcagcgtcgtgaacgagctggttccatacgaggtgcgtgatgtgctgttttccggcctcggctacctgcgttcacaaatttcatctcagcacacaatcatcatcgaggagactgagggctggtcccacaaccacgtctacaacgcggtgcgggcttaccttgcaacacgcatcaacaacaacatgcagcgcctgcgagtcagcagcatggatgaatcttccgagaagatggttgtcaccatggaggaaggtgaagagctggttgatatgcatgagggaacagaattcaaatggtgcttaatctcacgtagcatttcagctgaccccaacaatggcaatggcagcggccaacgtgaggtccgctcctatgagctgagcttccacaggaagcacaaggagaaagccctgaaatcatacctcccattcatcattgctacagccaaggccataaaagaccaggaaagaattctccagatatacatgaatgaatactcagactcatggtctccaattgatctccaccacccatccacattcgacacgcttgccatggaccagaagctgaaacagtcaattattgacgaccttgataggttcatcaagagaaaagattactacaagaggattggcaaggcatggaagaggggttacctgctgtatggtccaccagggactggcaagtccagcttgattgcagccatggcgaatcatctcaagtttgacatatatgatcttgagctgactggggtccattccaactcggagctcagaaggcttctagtcggaatgaccagccggtccattcttgttgttgaggacattgactgtagcatcgaactgaaacaacgggaggcaggggaggaacgtaccaagtccaactctacagaagaagacaagggagaagacaaagtaacattatccgggctgctcaattttgttgatgggctgtggtcaacaagtggagaggaaaggatcatcgttttcacgaccaattacaaggagcgtcttgatcaagcacttatgcggcctggcaggatggacatgcacatccacatggggtactgcaccccagaggctttccggattcttgccagcaactaccactcgatcgactatcatgtcacatatccagagatcgaggagctgatcaaggaggtgatggtgacgcctgcggaggtcgctgaggctctcatgagaaatgatgatattgatgttgcactccttggtctactggagctcctaaagtcaaagataaaagatgccagcgagaccaaggctgaaagcaaggatgcaaataagcagacggaggagaataaagatagcaaagcgatggagaacaaaaatgactcctcaactgatgaatgcacttaggattgtggagtacaacaatgacaacaagaatg

SEQ ID NO: 22 - DNA, 1455 bp, Oryza sativa
atggcgtcctacgacaaggccatcgagtcatacaagaaggccatcacaaccgctgcatccgttgcagcgtctgtgatgctggtccgcagcgtcgtgaacgagctggttccatacgaggtgcgtgatgtgctgttttccggcctcggctacctgcgttcacaaatttcatctcagcacacaatcatcatcgaggagactgagggctggtcccacaaccacgtctacaacgcggtgcgggcttaccttgcaacacgcatcaacaacaacatgcagcgcctgcgagtcagcagcatggatgaatcttccgagaagatggttgtcaccatggaggaaggtgaagagctggttgatatgcatgagggaacagaattcaaatggtgcttaatctcacgtagcatttcagctgaccccaacaatggcaatggcagcggccaacgtgaggtccgctcctatgagctgagcttccacaggaagcacaaggagaaagccctgaaatcatacctcccattcatcattgctacagccaaggccataaaagaccaggaaagaattctccagatatacatgaatgaatactcagactcatggtctccaattgatctccaccacccatccacattcgacacgcttgccatggaccagaagctgaaacagtcaattattgacgaccttgataggttcatcaagagaaaagattactacaagaggattggcaaggcatggaagaggggttacctgctgtatggtccaccagggactggcaagtccagcttgattgcagccatggcgaatcatctcaagtttgacatatatgatcttgagctgactggggtccattccaactcggagctcagaaggcttctagtcggaatgaccagccggtccattcttgttgttgaggacattgactgtagcatcgaactgaaacaacgggaggcaggggaggaacgtaccaagtccaactctacagaagaagacaagggagaagacaaagtaacattatccgggctgctcaattttgttgatgggctgtggtcaacaagtggagaggaaaggatcatcgttttcacgaccaattacaaggagcgtcttgatcaagcacttatgcggcctggcaggatggacatgcacatccacatggggtactgcaccccagaggctttccggattcttgccagcaactaccactcgatcgactatcatgtcacatatccagagatcgaggagctgatcaaggaggtgatggtgacgcctgcggaggtcgctgaggctctcatgagaaatgatgatattgatgttgcactccttggtctactggagctcctaaagtcaaagataaaagatgccagcgagaccaaggctgaaagcaaggatgcaaataagcagacggaggagaataaagatagcaaagcgatggagaacaaaaatgactcctcaactgatgaatgcacttag

SEQ ID NO: 23 - protein, 484 aa, Oryza sativa
MASYDKAIESYKKAITTAASVAASVMLVRSVVNELVPYEVRDVLFSGLGYLRSQISSQHTIIIEETEGWSHNHVYNAVRAYLATRINNNMQRLRVSSMDESSEKMVVTMEEGEELVDMHEGTEFKWCLISRSISADPNNGNGSGQREVRSYELSFHRKHKEKALKSYLPFIIATAKAIKDQERILQIYMNEYSDSWSPIDLHHPSTFDTLAMDQKLKQSIIDDLDRFIKRKDYYKRIGKAWKRGYLLYGPPGTGKSSLIAAMANHLKFDIYDLELTGVHSNSELRRLLVGMTSRSILVVEDIDCSIELKQREAGEERTKSNSTEEDKGEDKVTLSGLLNFVDGLWSTSGEERIIVFTTNYKERLDQALMRPGRMDMHIHMGYCTPEAFRILASNYHSIDYHVTYPEIEELIKEVMVTPAEVAEALMRNDDIDVALLGLLELLKSKIKDASETKAESKDANKQTEENKDSKAMENKNDSSTDECT

SEQ ID NO: 24 - DNA, 163 bp, Oryza sativa
tgatgttgcactccttggtctactggagctcctaaagtcaaagataaaagatgccagcgagaccaaggctgaaagcaaggatgcaaataagcagacggaggagaataaagatagcaaagcgatggagaacaaaaatgactcctcaactgatgaatgcacttag

SEQ ID NO: 25 - DNA, 199 bp, Lycopersicon esculentum
gtacggaccgtactactctattcgtttcaatatatttatttgtttcagctgactgcaagattcaaaaatttctttattattttaaattttgtgtcactcaaaaccagataaacaatttgatatagaggcactatatatatacatattctcgattatatatgtaaatgagttaacctttttttccacttaaattatatag

SEQ ID NO: 26 - DNA, 30 bp, Artificial Sequence
  Forward primer for cloning gDNA of OsDN-DTP2 gene
catggatccgattcaacacaaagaggcaac

SEQ ID NO: 27 - DNA, 36 bp, Artificial Sequence
  Reverse primer for cloning gDNA of OsDN-DTP2 gene
acactcgaggtatttgtctgcaatcctcatgtctag

SEQ ID NO: 28 - DNA, 24 bp, Artificial Sequence
  Forward primer for cloning cDNA of OsGSTU35 gene
acgatgggtgaaagggtgaagctc

SEQ ID NO: 29 - DNA, 31 bp, Artificial Sequence
  Reverse primer for cloning cDNA of OsGSTU35 gene
gaatcaaatagtaacttattccattcccatg

SEQ ID NO: 30 - DNA, 24 bp, Artificial Sequence
  Forward primer for cloning cDNA of OsCML1 gene
tctcccattcgagcgagatgaagc

SEQ ID NO: 31 - DNA, 26 bp, Artificial Sequence
  Reverse primer for cloning cDNA of OsCML1 gene
gaacggaggaatggatcaccacgatc

SEQ ID NO: 32 - DNA, 22 bp, Artificial Sequence
  Forward primer for cloning cDNA of OsIMPA1a gene
gcacgaggctggggatgacatg

SEQ ID NO: 33 - DNA, 26 bp, Artificial Sequence
  Reverse primer for cloning cDNA of OsIMPA1a gene
caaccaagactccaacgacaagactc

SEQ ID NO: 34 - DNA, 45 bp, Artificial Sequence
  Forward primer for cloning cDNA of OsMYB125 gene
atgatgtaccatgcaaagaagttctctgtaccctttggaccgcag

SEQ ID NO: 35 - DNA, 25 bp, Artificial Sequence
  Reverse primer for cloning cDNA of OsMYB125 gene
cgatcggcccgcagtggaggttaac

SEQ ID NO: 36 - DNA, 31 bp, Artificial Sequence
  Forward primer for cloning cDNA of OsCML3 gene
cttgtgttactaataatctttgaggggaggc

SEQ ID NO: 37 - DNA, 28 bp, Artificial Sequence
  Reverse primer for cloning cDNA of OsCML3 gene
ccagaacaagtgtaaccagaaattgagg

SEQ ID NO: 38 - DNA, 26 bp, Artificial Sequence
  Forward primer for cloning cDNA of OsBCS1L gene
ctcaccctccccattcaacactactg

SEQ ID NO: 39 - DNA, 28 bp, Artificial Sequence
  Reverse primer for cloning cDNA of OsBCS1L gene
cattcttgttgtcattgttgtactccac

SEQ ID NO: 40 - DNA, 17 bp, Artificial Sequence
  Forward primer for cloning cDNA fragment of OsBCS1L gene
tgatgttgcactccttg

SEQ ID NO: 41 - DNA, 20 bp, Artificial Sequence
  Reverse primer for cloning cDNA fragment of OsBCS1L gene
ctaagtgcattcatcagttg

SEQ ID NO: 42 - DNA, 26 bp, Artificial Sequence
  Forward primer for cloning sense strand cDNA of OsBCS1L gene for constructing RNAi vector
ctgctgaggtgatgttgcactccttg

SEQ ID NO: 43 - DNA, 31 bp, Artificial Sequence
  Reverse primer for cloning sense strand cDNA of OsBCS1L gene for constructing RNAi vector
gcttgctgaggctaagtgcattcatcagttg

SEQ ID NO: 44 - DNA, 26 bp, Artificial Sequence
  Forward primer for cloning antisense strand cDNA of OsBCS1L gene for constructing RNAi vector
ccgctgaggtgatgttgcactccttg

SEQ ID NO: 45 - DNA, 31 bp, Artificial Sequence
  Reverse primer for cloning antisense strand cDNA of OsBCS1L gene for constructing RNAi vector
gcaggctgaggctaagtgcattcatcagttg

SEQ ID NO: 46 - DNA, 20 bp, Artificial Sequence
  Forward primer for real-time RT-PCR analysis of OsDN-DTP2 gene
cctcattgcaaatcactggg

SEQ ID NO: 47 - DNA, 22 bp, Artificial Sequence
  Reverse primer for real-time RT-PCR analysis of OsDN-DTP2 gene
gacaaggaggactgcaggatag

SEQ ID NO: 48 - DNA, 20 bp, Artificial Sequence
  Forward primer for real-time RT-PCR analysis of OsGSTU35 gene
atttctggatcccgttcgtg

SEQ ID NO: 49 - DNA, 21 bp, Artificial Sequence
  Reverse primer for real-time RT-PCR analysis of OsGSTU35 gene
agattctcctttgcttccctc

SEQ ID NO: 50 - DNA, 18 bp, Artificial Sequence
  Forward primer for real-time RT-PCR analysis of OsCML1 gene
atggaggcgttcaaggtg

SEQ ID NO: 51 - DNA, 18 bp, Artificial Sequence
  Reverse primer for real-time RT-PCR analysis of OsCML1 gene
gaggatggcgaccatgac

SEQ ID NO: 52 - DNA, 19 bp, Artificial Sequence
  Forward primer for real-time RT-PCR analysis of OsIMPA1a gene
atgatgctgagggactgga

SEQ ID NO: 53 - DNA, 19 bp, Artificial Sequence
  Reverse primer for real-time RT-PCR analysis of OsIMPA1a gene
aagccgttttgagcgttgt

SEQ ID NO: 54 - DNA, 20 bp, Artificial Sequence
  Forward primer for real-time RT-PCR analysis of OsMYB125 gene
ctaccgcattcaccaccaag

SEQ ID NO: 55 - DNA, 20 bp, Artificial Sequence
  Reverse primer for real-time RT-PCR analysis of OsMYB125 gene
ggaatgcagcctcttgatcc

SEQ ID NO: 56 - DNA, 21 bp, Artificial Sequence
  Forward primer for real-time RT-PCR analysis of OsCML3 gene
gtcttcgacaaggaccagaac

SEQ ID NO: 57 - DNA, 20 bp, Artificial Sequence
  Reverse primer for real-time RT-PCR analysis of OsCML3 gene
ttgtagttgatctggccgtc

SEQ ID NO: 58 - DNA, 20 bp, Artificial Sequence
  Forward primer for real-time RT-PCR analysis of OsBCS1L gene
ccttggtctactggagctcc

SEQ ID NO: 59 - DNA, 21 bp, Artificial Sequence
  Reverse primer for real-time RT-PCR analysis of OsBCS1L gene
gttctccatcgctttgctatc

SEQ ID NO: 60 - DNA, 22 bp, Artificial Sequence
  Forward primer for real-time RT-PCR analysis of OsBCS1L gene in DP1200 transgenic rice
gattcttgccagcaactaccac

SEQ ID NO: 61 - DNA, 22 bp, Artificial Sequence
  Reverse primer for real-time RT-PCR analysis of OsBCS1L gene in DP1200 transgenic rice
ccagtagaccaaggagtgcaac